(19) United States
(12) Patent Application Publication
(10) Pub. No.: US 2024/0400980 A1
(43) Pub. Date: Dec. 5, 2024

(54) PORCINE PLURIPOTENT STEM CELL CULTURE MEDIUM AND USE THEREOF

(71) Applicant: CHINA AGRICULTURAL UNIVERSITY, Beijing (CN)

(72) Inventors: Jianyong HAN, Beijing (CN); Minglei ZHI, Beijing (CN); Jinying ZHANG, Beijing (CN)

(21) Appl. No.: 18/690,908

(22) PCT Filed: Sep. 7, 2022
(86) PCT No.: PCT/CN2022/117466

§ 371 Date: Mar. 11, 2024

(30) Foreign Application Priority Data
Sep. 10, 2021 (CN) 202111061391.4

(51) Int. Cl. C12N 5/073

(52) U.S. Cl. CPC C12N 5/0603; C12N 2501/115; C12N 2501/16; C12N 2501/235; C12N 2501/415

(57) ABSTRACT

The present invention relates to a medium for porcine pluripotent stem cells and use thereof, specifically provides a medium comprising: a first component that is IWR-1-endo; a second component that is selected from WH-4-023 or A419259; and a third component that is selected from fibroblast growth factors. The medium is easy to support the establishment of stable porcine pluripotent stem cell lines, especially the porcine E8-10 pre-gastrulation epiblast stem cell lines (known as pgEpiSCs).

### RELATED APPLICATIONS

The present application is a U.S. National Phase of International Application Number PCT/CN2022/117466 filed Sep. 7, 2022, which claims priority to Chinese Application Number 202111061391.4 filed Sep. 10, 2021.

### INCORPORATION BY REFERENCE

The sequence listing provided in the file entitled PUS124007PCT.xml, which is an Extensible Markup Language (XML) file that was created on Mar. 11, 2024, and which comprises 68,968 bytes, is hereby incorporated by reference in its entirety.

### TECHNICAL FIELD

The present invention relates to the field of biotechnology, specifically to a medium for porcine pluripotent stem cells and use thereof.

### BACKGROUND

During embryonic development, the epiblast that is developed from the inner cell mass (ICM) of the embryo, produces all somatic and germ cell lineages, thereby forming a normal embryo (Chazaud and Yamanaka, 2016; Rossant and Tam, 2018). In terms of pluripotency, epiblast cells are an important source of pluripotent stem cells (PSCs), including mouse embryonic stem cells (ESCs) from the naïve epiblast (Boroviak et al., 2014; Evans and Kaufman, 1981; Martin, 1981; Ying et al., 2008) and epiblast stem cells (EpiSCs) derived from the late developmental epiblast (Bao et al., 2009; Brons et al., 2007; Tesar et al., 2007). Recently, formative (or “intermediate”) pluripotent stem cells have been successfully obtained from mouse pre-gastrulation epiblast cells (Kinoshita et al., 2021; Wang et al., 2021; Yu et al., 2021). Conventional human ESCs are derived from the ICM of the blastocyst and exhibit primed pluripotency characteristics similar to mouse EpiSCs (Tesar et al., 2007; Thomson et al., 1998), and human PSCs with naïve or formative pluripotent states can also be obtained (Gafni et al., 2013; Kinoshita et al., 2021; Theunissen et al., 2014).

Compared with many other animal models, pigs are very similar to humans in terms of embryonic development (Kobayashi et al., 2017; Zhu et al., 2021), anatomy (Niu et al., 2017; Yue et al., 2021), and physiology (Yan et al., 2018). Therefore, stable porcine PSCs from epiblast cells should be an excellent model for understanding the characteristics of human PSCs, which may provide valuable informations for modeling human development (Xu et al., 2020; Yan et al., 2018). Stable porcine PSCs combined with precise multi-gene editing technology will have a huge impact on biomedical research and agricultural animal breeding (Navarro et al., 2019; Park et al., 2021; Whitworth et al., 2016; Zhao et al., 2019).

Surprisingly, despite persistent and extensive attempts by scientists since the 1990s, no stable porcine PSC line for long-term passage has been isolated from ICMs or epiblast cells at different stages (Alberio et al., 2010; Choi et al., 2019; Gao et al., 2019; Haraguchi et al., 2012; Hou et al., 2016; Notarianni et al., 1990; Park et al., 2013; Vassiliev et al., 2010; Yuan et al., 2019; Zhang et al., 2019). It has been suggested that why human and mouse stem cell culture techniques have not been used in the pig for decades may be explained by the interspecific differences in embryogenesis and in signaling pathways for pluripotency regulation during early embryonic development (Liu et al., 2021; Ramos-Ibeas et al., 2019).

### SUMMARY OF THE INVENTION

In terms of the molecular basis for embryogenesis and its PSCs establishment, high-resolution transcriptome mapping studies in pigs lags far behind those currently performed in mice, humans, and non-human primates. However, large-scale studies based on single cell RNA sequencing (scRNA-seq) technology have been used to characterize early embryos of mice, humans, and monkeys, and to track lineage development and the transition from embryos to stem cells (Boroviak et al., 2014; Deng et al., 2014; Nakamura et al., 2016; Petropoulos et al., 2016; Pijuan-Sala et al., 2019; Stirparo et al., 2018; Tang et al., 2010; Yan et al., 2013; Zhou et al., 2019), while currently, single cell transcriptome studies on porcine embryos do not provide accurate and high-resolution transcriptome maps for pre-implantation embryos due to limitations in the embryonic stage and the number of cells (Cao et al., 2014; Kong et al., 2020; Liu et al., 2021; Ramos-Ibeas et al., 2019; Tam and Ho, 2020; Wei et al., 2018).

For this purpose, the inventors collect porcine pre-implantation embryos, which are used for scRNA seq at all stages (embryos daily during E0-E14) to comprehensively analyze the molecular basis for early development and pluripotency changes in porcine embryos. Subsequently, a medium (called 3i/LAF) is developed by the inventors based on the analysis results, by which it is easy to establish stable porcine E8-10 pre-gastrulation epiblast stem cell lines (called pgEpiSCs). Extensive studies have shown that these pgEpiSCs possess pluripotency and molecular characteristics of pre-gastrulation epiblast cells, with a raised dome morphology, express pluripotency markers, maintain stability over 200 passages, and have the ability of efficient teratoma formation and differentiation into different types of cells. The inventors also realized consecutive rounds of gene editing, followed by nuclear transfer using gene-edited donor cells, which successfully produced homozygous edited piglets.

In view of this, in a first aspect of the present invention, a medium is provided, comprising:
a first component that is IWR-1-endo;
a second component that is selected from WH-4-023, A419259; and
a third component that is selected from fibroblast growth factors.

In some embodiments, the medium further comprises:
a fourth component that is selected from CHIR99021, WNT3a;
a fifth component that is selected from TGF-β superfamily members; and
a sixth component that is LIF.

In some embodiments, the second component is WH-4-023.

In some embodiments, the third component is selected from FGF2, FGF1.

In some embodiments, the third component is FGF2.

In some embodiments, the third component is recombinant human FGF2.

In some embodiments, the fourth component is CHIR99021.

In some embodiments, the fifth component is selected from Activin A, Nodal.

In some embodiments, the fifth component is Activin A.

In some embodiments, the fifth component is recombinant human Activin A.

In some embodiments, the sixth component is selected from recombinant human LIF, recombinant mouse LIF.

In some embodiments, the sixth component is recombinant human LIF.

In some embodiments, the first component has a concentration of 0.1-10 μM, for example, 0.1 μM, 0.2 μM, 0.3 μM, 0.4 μM, 0.5 μM, 0.7 μM, 0.9 μM, 1.1 μM, 1.3 μM, 1.5 μM, 1.7 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 5.1 μM, 5.3 μM, 5.5 μM, 5.7 μM, 5.9 μM, 6.0 μM, 6.1 μM, 6.3 μM, 6.5 μM, 6.7 μM, 6.9 μM, 7.0 μM, 7.1 μM, 7.3 μM, 7.5 μM, 7.7 μM, 7.9 μM, 8.0 μM, 8.1 μM, 8.3 μM, 8.5 μM, 8.7 μM, 8.9 μM, 9.0 μM, 9.1 μM, 9.3 μM, 9.5 μM, 9.7 μM, 9.9 μM or 10 μM, or 0.1-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM, 2.9-3.0 μM, 3.0-3.1 μM, 3.1-3.3 μM, 3.3-3.5 μM, 3.5-3.7 μM, 3.7-3.9 μM, 3.9-4.0 μM, 4.0-4.1 μM, 4.1-4.3 μM, 4.3-4.5 μM, 4.5-4.7 μM, 4.7-4.9 μM, 4.9-5.0 μM, 5.0-5.5 μM, 5.5-6 μM, 6-6.5 μM, 6.5-7 μM, 7-7.5 μM, 7.5-8 μM, 8-8.5 μM, 8.5-9 μM, 9-9.5 μM or 9.5-10 μM.

In some embodiments, the first component has a concentration of 0.9-3 μM.

In some embodiments, the first component has a concentration of 1-3 μM, for example, 1 μM, 1.1 μM, 1.3 μM, 1.5 μM, 1.7 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM or 3.0 μM or 1-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM or 2.9-3.0 μM.

In some embodiments, the first component has a concentration of 2.5 μM.

In some embodiments, the second component has a concentration of 3 nM-30 μM, for example, 3 nM, 4 nM, 5 nM, 6 nM, 7 nM, 8 nM, 9 nM, 0.01 μM, 0.03 μM, 0.05 μM, 0.07 UM, 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 7.0 μM, 10 μM, 13 μM, 15 μM, 17 μM, 20 μM, 23 μM, 25 μM, 27 μM or 30 μM, or 3-4 nM, 4-5 nM, 5-6 nM, 6-7 nM, 7-8 nM, 8-9 nM, 9-10 nM, 0.01-0.03 μM, 0.03-0.05 μM, 0.05-0.07 μM, 0.07-0.1 μM, 0.1-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM, 2.9-3.0 μM, 3.0-3.1 μM, 3.1-3.3 μM, 3.3-3.5 μM, 3.5-3.7 μM, 3.7-3.9 M, 3.9-4.0 μM, 4.0-4.1 μM, 4.1-4.3 μM, 4.3-4.5 μM, 4.5-4.7 M, 4.7-4.9 μM, 4.9-5.0 μM, 5.0-7.0 μM, 7-10 μM, 10-13 μM, 13-15 μM, 15-17 μM, 17-20 UM, 20-23 μM, 23-25 μM, 25-27 UM or 27-30 μM.

In some embodiments, the second component has a concentration of 0.01-5 μM, for example, 0.01 μM, 0.03 μM, 0.05 UM, 0.07 UM, 0.1 μM, 0.2 μM, 0.3 μM, 0.4 μM, 0.5 μM, 0.6 μM, 0.7 μM, 0.8 UM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 UM, 2.9 μM, 3.0 UM, 3.1 μM, 3.3 μM, 3.5 μM, 3.7 μM, 3.9 μM, 4.0 μM, 4.1 μM, 4.3 μM, 4.5 μM, 4.7 μM, 4.9 μM or 5.0 μM, or 0.01-0.03 μM, 0.03-0.05 UM, 0.05-0.07 UM, 0.07-0.1 μM, 0.1-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 M, 2.0-2.1 μM, 2.1-2.3 μM, 2.3-2.5 μM, 2.5-2.7 μM, 2.7-2.9 μM, 2.9-3.0 μM, 3.0-3.1 μM, 3.1-3.3 M, 3.3-3.5 μM, 3.5-3.7 μM, 3.7-3.9 μM, 3.9-4.0 μM, 4.0-4.1 μM, 4.1-4.3 μM, 4.3-4.5 μM, 4.5-4.7 μM, 4.7-4.9 μM or 4.9-5.0 μM.

In some embodiments, the second component has a concentration of 1 μM.

In some embodiments, the third component has a concentration of 0.01-100 ng/ml, for example, 0.01 ng/ml, 0.1 ng/ml, 0.2 ng/ml, 0.3 ng/mL, 0.4 ng/ml, 0.5 ng/ml, 0.6 ng/ml, 0.7 ng/mL, 0.8 ng/mL, 0.9 ng/mL, 1 ng/mL, 1.5 ng/ml, 2 ng/ml, 3 ng/mL, 4 ng/ml, 5 ng/mL, 6 ng/ml, 7 ng/ml, 8 ng/mL, 9 ng/ml, 10 ng/ml, 11 ng/ml, 12 ng/ml, 13 ng/ml, 14 ng/ml, 15 ng/ml, 16 ng/mL, 17 ng/ml, 18 ng/ml, 19 ng/ml, 20 ng/mL, 25 ng/ml, 30 ng/ml, 35 ng/ml, 40 ng/mL, 45 ng/mL, 50 ng/mL, 55 ng/mL, 60 ng/mL, 65 ng/ml, 70 ng/mL, 75 ng/ml, 80 ng/ml, 85 ng/ml, 90 ng/mL, 95 ng/mL or 100 ng/mL, or 0.01-0.1 ng/ml, 0.1-0.2 ng/ml, 0.2-0.5 ng/ml, 0.5-1 ng/ml, 1-1.5 ng/ml, 1.5-2 ng/ml, 2-3 ng/ml, 3-4 ng/ml, 4-5 ng/ml, 5-6 ng/ml, 6-7 ng/ml, 7-8 ng/ml, 8-9 ng/ml, 9-10 ng/ml, 10-11 ng/ml, 11-12 ng/ml, 12-13 ng/ml, 13-14 ng/mL, 14-15 ng/ml, 15-16 ng/ml, 16-17 ng/ml, 17-18 ng/ml, 18-19 ng/ml, 19-20 ng/mL, 20-25 ng/ml, 25-30 ng/mL, 30-35 ng/mL, 35-40 ng/mL, 40-45 ng/mL, 45-50 ng/mL, 50-55 ng/mL, 55-60 ng/mL, 60-65 ng/ml, 65-70 ng/mL, 70-75 ng/mL, 75-80 ng/mL, 80-85 ng/mL, 85-90 ng/ml, 90-95 ng/ml or 95-100 ng/mL.

In some embodiments, the third component has a concentration of 1-100 ng/ml.

In some embodiments, the third component has a concentration of 10 ng/ml.

In some embodiments, the fourth component has a concentration of 0.0025 nM-3 μM, for example, 0.0025 nM, 0.005 nM, 0.01 nM, 0.015 nM, 0.02 nM, 0.025 nM, 0.03 nM, 0.035 nM, 0.04 nM, 0.045 nM, 0.1 nM, 0.15 nM, 0.2 nM, 0.25 nM, 0.3 nM, 0.35 nM, 0.4 nM, 0.45 nM, 0.5 nM, 1 nM, 1.5 nM, 2 nM, 2.5 nM, 3 nM, 4 nM, 5 nM, 6 nM, 7 nM, 8 nM, 9 nM, 0.01 μM, 0.05 μM, 0.1 μM, 0.15 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM or 3.0 μM, or 0.01-0.05 μM, 0.05-0.1 μM, 0.1-0.15 μM, 0.15-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM or 2.9-3.0 μM.

In some embodiments, the fourth component has a concentration of 0.01-3 μM, for example, 0.01 μM, 0.05 μM, 0.1 μM, 0.15 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM or 3.0 μM, or 0.01-0.05 μM, 0.05-0.1 μM, 0.1-0.15 μM, 0.15-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM or 2.9-3.0 μM.

In some embodiments, the fourth component has a concentration of 1 μM.

In some embodiments, the fifth component has a concentration of 0.01-100 ng/ml, for example, 0.01 ng/ml, 0.5 ng/ml, 1 ng/ml, 1.5 ng/ml, 2 ng/ml, 2.5 ng/ml, 3 ng/ml, 3.5 ng/ml, 4 ng/ml, 4.5 ng/ml, 5 ng/ml, 6 ng/ml, 7 ng/mL, 8 ng/ml, 9 ng/ml, 10 ng/ml, 11 ng/ml, 12 ng/mL, 13 ng/ml, 14 ng/mL, 15 ng/ml, 16 ng/mL, 17 ng/ml, 18 ng/ml, 19 ng/ml, 20 ng/ml, 21 ng/ml, 22 ng/ml, 23 ng/mL, 24 ng/mL, 25 ng/mL, 26 ng/ml, 27 ng/ml, 28 ng/mL, 29 ng/ml, 30 ng/ml, 31 ng/mL, 32 ng/ml, 33 ng/ml, 34 ng/mL, 35 ng/ml, 36 ng/ml, 37 ng/mL, 38 ng/ml, 39 ng/mL, 40 ng/mL, 41 ng/mL, 42 ng/mL, 43 ng/mL, 44 ng/mL, 45 ng/mL, 46 ng/ml, 47 ng/ml, 48 ng/ml, 49 ng/mL, 50 ng/mL, 55 ng/mL, 60 ng/ml, 65 ng/mL, 70 ng/mL, 75 ng/ml, 80 ng/mL, 85 ng/mL, 90 ng/mL, 95 ng/ml or 100 ng/mL, or 0.01-0.5 ng/mL, 0.5-1 ng/ml, 1-2 ng/ml, 2-3 ng/mL, 3-4 ng/ml, 4-5 ng/mL, 5-6 ng/ml, 6-7 ng/ml, 7-8 ng/ml, 8-9 ng/ml, 9-10 ng/mL, 10-11 ng/mL, 11-12 ng/mL, 12-13 ng/ml, 13-14 ng/mL, 14-15 ng/mL, 15-16 ng/ml, 16-17 ng/ml, 17-18 ng/mL, 18-19 ng/ml, 19-20 ng/ml, 20-21 ng/ml, 21-23 ng/ml, 23-25 ng/ml, 25-27 ng/ml, 27-29 ng/mL, 29-30 ng/mL, 30-31 ng/ml, 31-33 ng/mL, 33-35 ng/ml, 35-37 ng/ml, 37-39 ng/ml, 39-40 ng/mL, 40-41 ng/mL, 41-43 ng/mL, 43-45 ng/mL, 45-47 ng/mL, 47-49 ng/mL, 49-50 ng/ml, 50-55 ng/ml, 55-60 ng/mL, 60-65 ng/mL, 65-70 ng/ml, 70-75 ng/mL, 75-80 ng/mL, 80-85 ng/mL, 85-90 ng/ml, 90-95 ng/mL or 95-100 ng/mL.

In some embodiments, the fifth component has a concentration of 25 ng/mL.

In some embodiments, the sixth component has a concentration of 0.01-100 ng/ml, for example, 0.01 ng/ml, 0.05 ng/ml, 0.1 ng/ml, 0.5 ng/mL, 0.7 ng/ml, 1 ng/mL, 2 ng/ml, 3 ng/ml, 4 ng/ml, 5 ng/ml, 6 ng/ml, 7 ng/ml, 8 ng/ml, 9 ng/ml, 10 ng/ml, 11 ng/mL, 12 ng/ml, 13 ng/mL, 14 ng/ml, 15 ng/ml, 16 ng/ml, 17 ng/mL, 18 ng/mL, 19 ng/ml, 20 ng/ml, 21 ng/ml, 22 ng/ml, 23 ng/ml, 24 ng/ml, 25 ng/ml, 26 ng/mL, 27 ng/ml, 28 ng/mL, 29 ng/ml, 30 ng/ml, 31 ng/mL, 32 ng/ml, 33 ng/ml, 34 ng/mL, 35 ng/mL, 36 ng/ml, 37 ng/ml, 38 ng/ml, 39 ng/ml, 40 ng/mL, 41 ng/mL, 42 ng/mL, 43 ng/mL, 44 ng/mL, 45 ng/ml, 46 ng/ml, 47 ng/ml, 48 ng/ml, 49 ng/mL, 50 ng/mL, 53 ng/mL, 55 ng/ml, 57 ng/ml, 60 ng/ml, 63 ng/mL, 65 ng/ml, 67 ng/ml, 70 ng/mL, 73 ng/mL, 75 ng/ml, 77 ng/ml, 80 ng/ml, 83 ng/ml, 85 ng/ml, 87 ng/ml, 90 ng/ml, 93 ng/mL, 95 ng/ml, 97 ng/mL or 100 ng/mL, or 0.01-0.05 ng/mL, 0.05-0.1 ng/mL, 0.1-0.5 ng/mL, 0.5-0.7 ng/ml, 0.7-1 ng/ml, 1-2 ng/ml, 2-3 ng/ml, 3-4 ng/ml, 4-5 ng/ml, 5-6 ng/ml, 6-7 ng/mL, 7-8 ng/mL, 8-9 ng/mL, 9-10 ng/mL, 10-11 ng/mL, 11-12 ng/ml, 12-13 ng/ml, 13-14 ng/ml, 14-15 ng/ml, 15-16 ng/ml, 16-17 ng/ml, 17-18 ng/ml, 18-19 ng/ml, 19-20 ng/ml, 20-21 ng/ml, 21-23 ng/ml, 23-25 ng/mL, 25-27 ng/mL, 27-29 ng/ml, 29-30 ng/mL, 30-31 ng/ml, 31-33 ng/ml, 33-35 ng/mL, 35-37 ng/ml, 37-39 ng/ml, 39-40 ng/mL, 40-41 ng/mL, 41-43 ng/mL, 43-45 ng/ml, 45-47 ng/ml, 47-49 ng/mL, 49-50 ng/mL, 50-53 ng/mL, 53-55 ng/mL, 55-57 ng/ml, 57-59 ng/mL, 59-60 ng/ml, 60-63 ng/ml, 63-65 ng/mL, 65-67 ng/ml, 67-69 ng/ml, 69-70 ng/ml, 70-73 ng/mL, 73-75 ng/ml, 75-77 ng/ml, 77-79 ng/mL, 79-80 ng/mL, 80-83 ng/mL, 83-85 ng/ml, 85-87 ng/ml, 87-90 ng/ml, 90-93 ng/ml, 93-95 ng/ml, 95-97 ng/ml, 97-99 ng/ml or 99-100 ng/mL.

In some embodiments, the sixth component has a concentration of 1-100 ng/mL.

In some embodiments, the sixth component has a concentration of 10 ng/ml.

In some embodiments, the concentration ratio of the fourth component to the first component is 25:1-1:25, for example, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24 or 1:25, or 25:1-23:1, 23:1-21:1, 21:1-20:1, 20:1-19:1, 19:1-17:1, 17:1-15:1, 15:1-13:1, 13:1-11:1, 11:1-10:1, 10:1-9:1, 9:1-7:1, 7:1-5:1, 5:1-3:1, 3:1-1:1, 1:1-1:3, 1:3-1:5, 1:5-1:7, 1:7-1:9, 1:9-1:10, 1:10-1:11, 1:11-1:13, 1:13-1:15, 1:15-1:17, 1:17-1:19, 1:19-1:20, 1:20-1:21, 1:21-1:23 or 1:23-1:25.

In some embodiments, the concentration ratio of the fourth component to the first component is 2:3-1:3.

In some embodiments, the concentration ratio of the fourth component to the first component is 1:2-1:3.

In some embodiments, the medium comprises:

| Name | Concentration | |
|---|---|---|
| CHIR99021 | 1 | μM |
| IWR-1-endo | 2.5 | μM |
| WH-4-023 | 1 | μM |
| Recombinant human Activin A | 25 | ng/mL |
| Recombinant human FGF2 | 10 | ng/mL |
| Recombinant human LIF | 10 | ng/mL |

It should be noted that the concentrations of the first, second, third, fourth, fifth, and sixth components above all refer to the final concentration of each component in the medium.

In some embodiments, the medium further comprises a seventh component that is a ROCK inhibitor. The addition of ROCK inhibitors such as Y-27632 can promote the proliferation of pgEpiSCs.

In some embodiments, the seventh component is Y-27632.

In some embodiments, the seventh component has a concentration of 0.01-50 μM, for example, 0.01 μM, 0.05 μM, 0.1 μM, 0.3 μM, 0.5 μM, 0.7 μM, 0.9 μM, 1 μM, 1.1 μM, 1.3 μM, 1.5 μM, 1.7 μM, 1.9 M, 2.0 μM, 2.1 μM, 2.3 μM, 2.5 μM, 2.7 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 5.1 μM, 5.3 μM, 5.5 μM, 5.7 μM, 5.9 μM, 6.0 μM, 6.1 μM, 6.3 μM, 6.5 μM, 6.7 μM, 6.9 μM, 7.0 μM, 7.1 μM, 7.3 μM, 7.5 μM, 7.7 μM, 7.9 μM, 8.0 μM, 8.1 μM, 8.3 μM, 8.5 μM, 8.7 μM, 8.9 μM, 9.0 μM, 9.1 μM, 9.3 μM, 9.5 μM, 9.7 μM, 9.9 μM, 10 μM, 10.1 μM, 10.3 μM, 10.5 μM, 10.7 μM, 10.9 μM, 11 μM, 11.1 μM, 11.3 μM, 11.5 μM, 11.7 μM, 11.9 μM, 12 μM, 12.1 μM, 12.3 μM, 12.5 μM, 12.7 μM, 12.9 μM, 13 μM, 13.1 μM, 13.3 μM, 13.5 μM, 13.7 μM, 13.9 μM, 14 μM, 14.1 μM, 14.3 μM, 14.5 μM, 14.7 μM, 14.9 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, 25 μM, 25.5 μM, 26 μM, 26.5 μM, 27 μM, 27.5 μM, 28 μM, 28.5 μM, 29 μM, 29.5 μM, 30 μM, 30.5 μM, 31 μM, 31.5 μM, 32 μM, 32.5 μM, 33 μM, 33.5 μM, 34 μM, 34.5 μM, 35 μM, 35.5 μM, 36 μM, 36.5 μM, 37 μM, 37.5 μM, 38 μM, 38.5 μM, 39 μM, 39.5 μM, 40 μM, 40.5 μM, 41 μM, 41.5 μM, 42 μM, 42.5 μM, 43 μM, 43.5 μM, 44 μM, 44.5 μM, 45 μM, 45.5 μM, 46 μM, 46.5 μM, 47 μM, 47.5 μM, 48 μM, 48.5 μM, 49 μM, 49.5 μM or 50 μM, or 0.01-0.05 μM, 0.05-0.1 μM, 0.1-0.5 μM, 0.5-1 μM, 1-1.5 μM, 1.5-2 μM, 2-2.5 μM, 2.5-3 μM, 3-3.5 μM, 3.5-4 μM, 4-4.5 μM, 4.5-5 μM, 5-5.5 μM, 5.5-6 μM, 6-6.5 μM, 6.5-7 μM, 7-7.5 μM, 7.5-8 μM, 8-8.5 μM, 8.5-9 μM, 9-9.5 μM, 9.5-10 μM, 10-10.5 μM, 10.5-11 μM, 11-11.5 μM, 11.5-12 μM, 12-12.5 μM, 12.5-13 μM, 13-13.5 μM, 13.5-14 μM, 14-14.5 μM, 14.5-15 μM, 15-20 μM, 20-23 μM, 23-25 μM, 25-27 μM, 27-30 μM, 30-33 μM, 33-35 μM, 35-37 μM, 37-40 μM, 40-43 μM, 43-45 μM, 45-47 μM or 47-50 μM.

In some embodiments, when the medium is used for cell passage, the seventh component has a concentration of 0.01-20 μM, for example, 0.01 μM, 0.05 μM, 0.1 μM, 0.3 μM, 0.5 μM, 0.7 μM, 0.9 μM, 1 μM, 1.1 μM, 1.3 μM, 1.5 μM, 1.7 μM, 1.9 μM, 2.0 μM, 2.1 μM, 2.3 μM, 2.5 μM, 2.7 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 5.1 μM, 5.3 μM, 5.5 μM, 5.7 μM, 5.9 μM, 6.0 μM, 6.1 μM, 6.3 μM, 6.5 μM, 6.7 μM, 6.9 μM, 7.0 μM, 7.1 μM, 7.3 μM, 7.5 μM, 7.7 μM, 7.9 μM, 8.0 μM, 8.1 μM, 8.3 μM, 8.5 μM, 8.7 μM, 8.9 μM, 9.0 μM, 9.1 μM, 9.3 μM, 9.5 μM, 9.7 μM, 9.9 μM, 10 μM, 10.1 μM, 10.3 μM, 10.5 μM, 10.7 μM, 10.9 μM, 11 μM, 11.1 μM, 11.3 μM, 11.5 μM, 11.7 μM, 11.9 μM, 12 μM, 12.1 μM, 12.3 μM, 12.5 μM, 12.7 μM, 12.9 μM, 13 μM, 13.1 μM, 13.3 μM, 13.5 μM, 13.7 μM, 13.9 μM, 14 μM, 14.1 μM, 14.3 μM, 14.5 μM, 14.7 μM, 14.9 μM, 15 μM, 15.5 μM, 16 μM, 16.5 μM, 17 μM, 17.5 μM, 18 μM, 18.5 μM, 19 μM, 19.5 μM or 20 μM, or 0.01-0.05 μM, 0.05-0.1 μM, 0.1-0.5 μM, 0.5-1 μM, 1-1.5 μM, 1.5-2 μM, 2-2.5 μM, 2.5-3 μM, 3-3.5 μM, 3.5-4 μM, 4-4.5 μM, 4.5-5 μM, 5-5.5 μM, 5.5-6 μM, 6-6.5 μM, 6.5-7 μM, 7-7.5 μM, 7.5-8 μM, 8-8.5 μM, 8.5-9 μM, 9-9.5 μM, 9.5-10 μM, 10-10.5 μM, 10.5-11 μM, 11-11.5 μM, 11.5-12 μM, 12-12.5 μM, 12.5-13 μM, 13-13.5 μM, 13.5-14 μM, 14-14.5 μM, 14.5-15 μM or 15-20 μM, preferably 10 μM.

In some embodiments, when the medium is used for cell maintenance, the seventh component has a concentration of 0.01-10 μM, for example, 0.01 μM, 0.05 μM, 0.1 μM, 0.3 μM, 0.5 μM, 0.7 μM, 0.9 μM, 1 μM, 1.1 μM, 1.3 μM, 1.5 μM, 1.7 μM, 1.9 μM, 2.0 μM, 2.1 μM, 2.3 μM, 2.5 μM, 2.7 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 5.1 μM, 5.3 μM, 5.5 M, 5.7 μM, 5.9 μM, 6.0 μM, 6.1 μM, 6.3 μM, 6.5 M, 6.7 μM, 6.9 μM, 7.0 μM, 7.1 μM, 7.3 μM, 7.5 μM, 7.7 μM, 7.9 μM, 8.0 μM, 8.1 μM, 8.3 μM, 8.5 μM, 8.7 μM, 8.9 μM, 9.0 μM, 9.1 μM, 9.3 μM, 9.5 μM, 9.7 μM, 9.9 μM or 10 μM, or 0.01-0.05 μM, 0.05-0.1 μM, 0.1-0.5 μM, 0.5-1 μM, 1-1.5 μM, 1.5-2 μM, 2-2.5 μM, 2.5-3 μM, 3-3.5 μM, 3.5-4 μM, 4-4.5 μM, 4.5-5 μM, 5-5.5 μM, 5.5-6 μM, 6-6.5 μM, 6.5-7 μM, 7-7.5 μM, 7.5-8 μM, 8-8.5 μM, 8.5-9 μM, 9-9.5 μM or 9.5-10 μM, preferably 2 μM.

It should be noted that the concentration of the seventh component above refers to its final concentration in the medium.

In some embodiments, the medium further comprises an eighth component that is a basal medium.

In some embodiments, the basal medium is used to culture mammalian (preferably porcine) pluripotent stem cells.

In some embodiments, the basal medium comprises a minimal medium, N2 supplement, B27 supplement, non-essential amino acids, β-mercaptoethanol, knockout serum replacement, and any one selected from GlutaMAX, glutamine.

In some embodiments, the basal medium comprises a minimal medium, N2 supplement, B27 supplement, non-essential amino acids, β-mercaptoethanol, knockout serum replacement and GlutaMAX.

In some embodiments, the basal medium comprises a minimal medium, N2 supplement, B27 supplement, non-essential amino acids, β-mercaptoethanol, knockout serum replacement, ascorbic acid, GlutaMAX and penicillin-streptomycin.

In some embodiments, the minimal medium is selected from DMEM/F12, Neurobasal, DMEM, KO-DMEM, RPMI1640, MEM, mTeSR1, or any combination thereof.

In some embodiments, the minimal medium is selected from DMEM/F12, Neurobasal, or a combination thereof.

In some embodiments, the minimal medium is DMEM/F12 and Neurobasal.

In some embodiments, the minimal medium has a volume fraction of 1%-99%, for example, 1%, 3%, 5%, 7%, 9%, 10%, 11%, 13%, 15%, 17%, 19%, 20%, 21%, 23%, 25%, 27%, 29%, 30%, 31%, 33%, 35%, 37%, 39%, 40%, 41%, 42%, 43%, 44%, 45%, 45.5%, 46%, 47%, 48%, 49%, 50%, 51%, 53%, 55%, 57%, 59%, 60%, 61%, 63%, 65%, 67%, 69%, 70%, 71%, 73%, 75%, 77%, 79%, 80%, 81%, 83%, 85%, 87%, 89%, 90%, 91%, 93%, 95%, 97% or 99%, or 1%-3%, 3%-5%, 5%-7%, 7%-9%, 9%-11%, 11%-13%, 13%-15%, 15%-17%, 17%-19%, 19%-20%, 20%-21%, 21%-23%, 23%-25%, 25%-27%, 27%-29%, 29%-30%, 30%-31%, 31%-33%, 33%-35%, 35%-37%, 37%-39%, 39%-40%, 40%-41%, 41%-42%, 42%-43%, 43%-44%, 44%-45%, 45%-45.5%, 45.5%-46%, 46%-47%, 47%-48%, 48%-49%, 49%-50%, 50%-51%, 51%-53%, 53%-55%, 55%-57%, 57%-59%, 59%-60%, 60%-61%, 61%-63%, 63%-65%, 65%-67%, 67%-69%, 69%-70%, 70%-71%, 71%-73%, 73%-75%, 75%-77%, 77%-79%, 79%-80%, 80%-81%, 81%-83%, 83%-85%, 85%-87%, 87%-89%, 89%-90%, 90%-91%, 91%-93%, 93%-95%, 95%-97% or 97%-99%.

In some embodiments, the minimal medium has a volume fraction of 91%.

In some embodiments, the DMEM/F12 has a volume fraction of 1%-99%, for example, 1%, 3%, 5%, 7%, 9%, 10%, 11%, 13%, 15%, 17%, 19%, 20%, 21%, 23%, 25%, 27%, 29%, 30%, 31%, 33%, 35%, 37%, 39%, 40%, 41%, 42%, 43%, 44%, 45%, 45.5%, 46%, 47%, 48%, 49%, 50%, 51%, 53%, 55%, 57%, 59%, 60%, 61%, 63%, 65%, 67%, 69%, 70%, 71%, 73%, 75%, 77%, 79%, 80%, 81%, 83%, 85%, 87%, 89%, 90%, 91%, 93%, 95%, 97% or 99%, or 1%-3%, 3%-5%, 5%-7%, 7%-9%, 9%-11%, 11%-13%, 13%-15%, 15%-17%, 17%-19%, 19%-20%, 20%-21%, 21%-23%, 23%-25%, 25%-27%, 27%-29%, 29%-30%, 30%-31%, 31%-33%, 33%-35%, 35%-37%, 37%-39%, 39%-40%, 40%-41%, 41%-42%, 42%-43%, 43%-44%, 44%-45%, 45%-45.5%, 45.5%-46%, 46%-47%, 47%-48%, 48%-49%, 49%-50%, 50%-51%, 51%-53%, 53%-55%, 55%-57%, 57%-59%, 59%-60%, 60%-61%, 61%-63%, 63%-65%, 65%-67%, 67%-69%, 69%-70%, 70%-71%, 71%-73%, 73%-75%, 75%-77%, 77%-79%, 79%-80%, 80%-81%, 81%-83%, 83%-85%, 85%-87%, 87%-89%, 89%-90%, 90%-91%, 91%-93%, 93%-95%, 95%-97% or 97%-99%.

In some embodiments, the DMEM/F12 has a volume fraction of 45%-50% (such as 45.5%, 46%, 46.5%).

In some embodiments, the Neurobasal has a volume fraction of 1%-99%, for example, 1%, 3%, 5%, 7%, 9%, 10%, 11%, 13%, 15%, 17%, 19%, 20%, 21%, 23%, 25%, 27%, 29%, 30%, 31%, 33%, 35%, 37%, 39%, 40%, 41%, 42%, 43%, 44%, 45%, 45.5%, 46%, 47%, 48%, 49%, 50%, 51%, 53%, 55%, 57%, 59%, 60%, 61%, 63%, 65%, 67%, 69%, 70%, 71%, 73%, 75%, 77%, 79%, 80%, 81%, 83%, 85%, 87%, 89%, 90%, 91%, 93%, 95%, 97% or 99%, or 1%-3%, 3%-5%, 5%-7%, 7%-9%, 9%-11%, 11%-13%, 13%-15%, 15%-17%, 17%-19%, 19%-20%, 20%-21%, 21%-23%, 23%-25%, 25%-27%, 27%-29%, 29%-30%, 30%-31%, 31%-33%, 33%-35%, 35%-37%, 37%-39%, 39%-40%, 40%-41%, 41%-42%, 42%-43%, 43%-44%, 44%-45%, 45%-45.5%, 45.5%-46%, 46%-47%, 47%-48%, 48%-49%, 49%-50%, 50%-51%, 51%-53%, 53%-55%, 55%-57%, 57%-59%, 59%-60%, 60%-61%, 61%-63%, 63%-65%, 65%-67%, 67%-69%, 69%-70%, 70%-71%, 71%-73%, 73%-75%, 75%-77%, 77%-79%, 79%-80%, 80%-81%, 81%-83%, 83%-85%, 85%-87%, 87%-89%, 89%-90%, 90%-91%, 91%-93%, 93%-95%, 95%-97% or 97%-99%.

In some embodiments, the Neurobasal has a volume fraction of 45%-50% (such as 45.5%, 46%, 46.5%).

In some embodiments, the N2 supplement has a volume fraction of 0.002%-10%, for example, 0.002%, 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.13%, 0.15%, 0.17%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9% or 10%, or 0.002%-0.05%, 0.05%-0.1%, 0.1%-0.15%, 0.15%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5% or 9.5%-10%.

In some embodiments, the N2 supplement has a volume fraction of 0.5%.

In some embodiments, the B27 supplement has a volume fraction of 0.002%-20%, for example, 0.002%, 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.13%, 0.15%, 0.17%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9%, 10%, 10.1%, 10.3%, 10.5%, 10.7%, 10.9%, 11%, 11.1%, 11.3%, 11.5%, 11.7%, 11.9%, 12%, 12.1%, 12.3%, 12.5%, 12.7%, 12.9%, 13%, 13.1%, 13.3%, 13.5%, 13.7%, 13.9%, 14%, 14.1%, 14.3%, 14.5%, 14.7%, 14.9%, 15%, 15.5%, 16%, 16.5%, 17%, 17.5%, 18%, 18.5%, 19%, 19.5% or 20%, or 0.002%-0.05%, 0.05%-0.1%, 0.1%-0.15%, 0.15%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5%, 9.5%-10%, 10%-10.5%, 10.5%-11%, 11%-11.5%, 11.5%-12%, 12%-12.5%, 12.5%-13%, 13%-13.5%, 13.5%-14%, 14%-14.5%, 14.5%-15% or 15%-20%.

In some embodiments, the B27 supplement has a volume fraction of 1%.

In some embodiments, the non-essential amino acids have a volume fraction of 0.01%-10%, for example, 0.01%, 0.05%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9% or 10%, or 0.01%-0.05%, 0.05%-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5% or 9.5%-10%.

In some embodiments, the non-essential amino acids have a volume fraction of 1%.

In some embodiments, the β-mercaptoethanol has a concentration of 0.01 mM-1 mM, for example, 0.01 mM, 0.02 mM, 0.03 mM, 0.04 mM, 0.05 mM, 0.06 mM, 0.07 mM, 0.08 mM, 0.09 mM, 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.53 mM, 0.55 mM, 0.57 mM, 0.59 mM, 0.6 mM, 0.61 mM, 0.63 mM, 0.65 mM, 0.67 mM, 0.69 mM, 0.7 mM, 0.71 mM, 0.73 mM, 0.75 mM, 0.77 mM, 0.79 mM, 0.8 mM, 0.81 mM, 0.83 mM, 0.85 mM, 0.87 mM, 0.89 mM, 0.9 mM, 0.91 mM, 0.93 mM, 0.95 mM, 0.97 mM, 0.99 mM or 1 mM, or 0.01-0.02 mM, 0.02-0.03 mM, 0.03-0.04 mM, 0.04-0.05 mM, 0.05-0.06 mM, 0.06-0.07 mM, 0.07-0.08 mM, 0.08-0.09 mM, 0.09-0.1 mM, 0.1-0.11 mM, 0.11-0.12 mM, 0.12-0.13 mM, 0.13-0.14 mM, 0.14-0.15 mM, 0.15-0.16 mM, 0.16-0.17 mM, 0.17-0.18 mM, 0.18-0.19 mM, 0.19-0.2 mM, 0.2-0.21 mM, 0.21-0.23 mM, 0.23-0.25 mM, 0.25-0.27 mM, 0.27-0.29 mM, 0.29-0.3 mM, 0.3-0.31 mM, 0.31-0.33 mM, 0.33-0.35 mM, 0.35-0.37 mM, 0.37-0.39 mM, 0.39-0.4 mM, 0.4-0.41 mM, 0.41-0.43 mM, 0.43-0.45 mM, 0.45-0.47 mM, 0.47-0.49 mM, 0.49-0.5 mM, 0.5-0.55 mM, 0.55-0.6 mM, 0.6-0.65 mM, 0.65-0.7 mM, 0.7-0.75 mM, 0.75-0.8 mM, 0.8-0.85 mM, 0.85-0.9 mM, 0.9-0.95 mM or 0.95-1 mM.

In some embodiments, the β-mercaptoethanol has a concentration of 0.1 mM.

In some embodiments, the knockout serum replacement has a volume fraction of 0.01%-50%, for example, 0.01%, 0.05%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9%, 10%, 10.1%, 10.3%, 10.5%, 10.7%, 10.9%, 11%, 11.1%, 11.3%, 11.5%, 11.7%, 11.9%, 12%, 12.1%, 12.3%, 12.5%, 12.7%, 12.9%, 13%, 13.1%, 13.3%, 13.5%, 13.7%, 13.9%, 14%, 14.1%, 14.3%, 14.5%, 14.7%, 14.9%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 35%, 40%, 45% or 50%, or 0.01%-0.05%, 0.05%-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5%, 9.5%-10%, 10%-10.5%, 10.5%-11%, 11%-11.5%, 11.5%-12%, 12%-12.5%, 12.5%-13%, 13%-13.5%, 13.5%-14%, 14%-14.5%, 14.5%-15%, 15%-20%, 20%-25%, 25%-30%, 30%-35%, 35%-40%, 40%-45% [symbol] 45%-50%.

In some embodiments, the knockout serum replacement has a volume fraction of 5%.

In some embodiments, the ascorbic acid has a concentration of 1 μg/mL-5000 μg/mL, for example, 1 μg/mL, 5 μg/mL, 10 μg/mL, 15 μg/mL, 20 μg/mL, 25 μg/mL, 30 μg/mL, 35 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 65 μg/mL, 70 μg/mL, 75 μg/mL, 80 μg/mL, 85 μg/mL, 90 μg/mL, 95 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 μg/mL, 260 μg/mL, 270 μg/mL, 280 μg/mL, 290 μg/mL, 300 μg/mL, 310 μg/mL, 320 μg/mL, 330 μg/mL, 340 μg/mL, 350 μg/mL, 360 μg/mL, 370 μg/mL, 380 μg/mL, 390 μg/mL, 400 μg/mL, 410 μg/mL, 420 μg/mL, 430 μg/mL, 440 μg/mL, 450 μg/mL, 460 μg/mL, 470 μg/mL, 480 μg/mL, 490 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, 1000 μg/mL, 2000 μg/mL, 3000 μg/mL, 4000 μg/mL or 5000 μg/mL, or 1-5 μg/mL, 5-10 μg/mL, 10-15 μg/mL, 15-20 μg/mL, 20-25 μg/mL, 25-30 μg/mL, 30-35 μg/mL, 35-40 μg/mL, 40-45 μg/mL, 45-50 μg/mL, 50-55 μg/mL, 55-60 μg/mL, 60-65 μg/mL, 65-70 μg/mL, 70-75 μg/mL, 75-80 μg/mL, 80-85 μg/mL, 85-90 μg/mL, 90-95 μg/mL, 95-100 μg/mL, 100-110 μg/mL, 110-120 μg/mL, 120-130 μg/mL, 130-140 μg/mL, 140-150 μg/mL, 150-170 μg/mL, 170-190 μg/mL, 190-200 μg/mL, 200-210 μg/mL, 210-230 μg/mL, 230-250 μg/mL, 250-270 μg/mL, 270-290 μg/mL, 290-300 μg/mL, 300-310 μg/mL, 310-330 μg/mL, 330-350 μg/mL, 350-370 μg/mL, 370-390 μg/mL, 390-400 μg/mL, 400-410 μg/mL, 410-430 μg/mL, 430-450 μg/mL, 450-470 μg/mL, 470-490 μg/mL, 490-500 μg/mL, 500-550 μg/mL, 550-600 μg/mL, 600-650 μg/mL, 650-700 μg/mL, 700-750 μg/mL, 750-800 μg/mL, 800-850 μg/mL, 850-900 μg/mL, 900-950 μg/mL, 950-1000 μg/mL, 1000-2000 μg/mL, 2000-3000 μg/mL, 3000-4000 μg/mL or 4000-5000 μg/mL.

In some embodiments, the ascorbic acid has a concentration of 50 μg/mL.

In some embodiments, the GlutaMAX or glutamine (preferably GlutaMAX) has a volume fraction of 0.01%-10%, for example, 0.01%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9% or 10%, or 0.01%-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5% or 9.5%-10%.

In some embodiments, the GlutaMAX or glutamine (preferably GlutaMAX) has a volume fraction of 0.5%.

In some embodiments, the penicillin-streptomycin has a volume fraction of 0.01%-20%, for example, 0.01%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9%, 10%, 10.1%, 10.3%, 10.5%, 10.7%, 10.9%, 11%, 11.1%, 11.3%, 11.5%, 11.7%, 11.9%, 12%, 12.1%, 12.3%, 12.5%, 12.7%, 12.9%, 13%, 13.1%, 13.3%, 13.5%, 13.7%, 13.9%, 14%, 14.1%, 14.3%, 14.5%, 14.7%, 14.9%, 15%, 15.5%, 16%, 16.5%, 17%, 17.5%, 18%, 18.5%, 19%, 19.5% or 20%, or 0.01-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5%, 9.5%-10%, 10%-10.5%, 10.5%-11%, 11%-11.5%, 11.5%-12%, 12%-12.5%, 12.5%-13%, 13%-13.5%, 13.5%-14%, 14%-14.5%, 14.5%-15% or 15%-20%. The addition of the penicillin-streptomycin is beneficial for preventing the cells from being infected.

In some embodiments, the penicillin-streptomycin has a volume fraction of 1%.

In some embodiments, the volume ratio of the DMEM/F12 to the Neurobasal is 5:1-1:5, for example, 5:1, 4:1, 3:1, 2:1, 1:1, 1:2, 1:3, 1:4 or 1:5, or 5:1-4:1, 4:1-3:1, 3:1-2:1, 2:1-1:1, 1:1-1:2, 1:2-1:3, 1:3-1:4 or 1:4-1:5.

In some embodiments, the volume ratio of the DMEM/F12 to the Neurobasal is 1:1.

It should be noted that the concentration of each specific component in the eighth component above all refer to their respective final concentrations in the medium. In addition, the volume fraction of each specific component in the eighth component above all refer to their respective volume/the total volume of the medium.

In some embodiments, per 500 mL of the medium comprises:

| | Concentration or volume | |
|---|---|---|
| Name | or volume fraction | |
| CHIR99021 | 1 | μM |
| IWR-1-endo | 2.5 | μM |
| WH-4-023 | 1 | μM |
| Recombinant human Activin A | 25 | ng/mL |
| Recombinant human FGF2 | 10 | ng/mL |
| Recombinant human LIF | 10 | ng/mL |
| DMEM/F12 | 227.5 | mL |
| Neurobasal | 227.5 | mL |
| N2 supplement | 2.5 | mL |
| B27 supplement | 5 | mL |
| GlutaMAX | Volume fraction of 0.5% | |
| Non-essential amino acids | Volume fraction of 1% | |
| β-mercaptoethanol | 0.1 | mM |
| Penicillin-streptomycin | Volume fraction of 1% | |
| Knockout serum replacement | Volume fraction of 5% | |
| Ascorbic acid | 50 | μg/mL |

| | |
|---|---|
| Y-27632 (optional) | 10 μM or 2 μM |

It should also be noted that the components or the specific ingredients in each component in the medium above are reagents commonly used by those skilled in the art and are commercially available.

In some embodiments, examples commercially available are as follows:

| Name | Brand, catalog number |
|---|---|
| DMEM/F12 | Thermo Fisher 729 Scientific, 10565-018 |
| Neurobasal | Thermo Fisher Scientific, 21103-049 |
| N2 supplement | Thermo Fisher Scientific, 17502-048 |
| B27 supplement | Thermo Fisher Scientific, 12587-010 |
| GlutaMAX | Thermo Fisher Scientific, 35050-061 |
| Non-essential amino acids | Thermo Fisher 732 Scientific, 11140-050 |
| β-mercaptoethanol | Thermo Fisher Scientific, 21985-023 |
| Penicillin-streptomycin | Thermo FisherScientific, 15140-122 |
| Knockout serum replacement | KOSR, Thermo Fisher Scientific, A3181502, optional |
| Ascorbic acid | Sigma-Aldrich, A4544 |
| CHIR99021 | Selleckchem, S1263 |
| IWR-1-endo | Selleckchem, S7086 |
| WH-4-023 | Selleckchem, S7565 |
| Recombinant human LIF | PeproTech, 300-05 |
| Recombinant human Activin A | Peprotech, 120-14E |
| Recombinant human FGF2 | Peprotech, 100-18B |
| Y-27632 | Selleckchem, S1049 |

In some embodiments, the non-essential amino acids comprise glycine, L-alanine, L-asparagine, L-aspartic acid, L-glutamic acid, L-proline, and L-serine.

In some embodiments, the non-essential amino acids comprise:

| Ingredients | Concentration (mg/L) | Concentration (mM) |
|---|---|---|
| Glycine | 750.0 | 10.0 |
| L-alanine | 890.0 | 10.0 |
| L-asparagine | 1320.0 | 10.0 |
| L-aspartic acid | 1330.0 | 10.0 |
| L-glutamic acid | 1470.0 | 10.0 |
| L-proline | 1150.0 | 10.0 |
| L-serine | 1050.0 | 10.0 |

It should be noted that in the above table, the concentration of each ingredient of the non-essential amino acids refers to the concentration of each specific ingredient in the non-essential amino acids.

In a second aspect of the present invention, a method for preparing mammalian pluripotent stem cells is provided, comprising:
1) providing a mammalian embryonic epiblast or its inner cell mass;
2) culturing the mammalian embryonic epiblast or its inner cell mass using the medium above to obtain mammalian pluripotent stem cells.

In some embodiments, the mammal is a pig.

In some embodiments, the mammalian embryonic epiblast is E8 to E10 (such as E8, E9 or E10) mammalian embryonic epiblast.

In some embodiments, the method is performed in the presence of feeder cells.

In some embodiments, the feeder cells are selected from mouse embryonic fibroblasts or STO cells.

In some embodiments, the feeder cells are mouse embryonic fibroblasts.

In some embodiments, the feeder cells are mouse embryonic fibroblasts with cell division arrest.

In some embodiments, the feeder cells are mouse embryonic fibroblasts treated with mitomycin C.

In some embodiments, the feeder cells have a density of 10⁴ cells/well to 10×10⁵ cells/well.

In some embodiments, the feeder cells have a density of 2×10⁴ cells/well to 2×10⁵ cells/well.

In some embodiments, the culture vessel is a 12-well plate.

In some embodiments, the method is performed at a temperature of 37-39° C. (preferably 37° C.), oxygen concentration of 5%-22% (preferably 5%), carbon dioxide concentration of 4%-6% (preferably 5%), and humidity of 100%.

In some embodiments, the medium is refreshed every 10-48 hours (preferably 10-24 hours, more preferably 12 hours) in the method.

In a third aspect of the present invention, there is provided a method for culturing mammalian pluripotent stem cells and/or maintaining their pluripotency, comprising:
1) providing mammalian pluripotent stem cells;
2) culturing the mammalian pluripotent stem cells using the medium above.

In some embodiments, the mammal is a pig.

In some embodiments, the mammalian pluripotent stem cells are porcine embryonic pre-gastrulation epiblast stem cells.

In some embodiments, the method is performed in the presence of feeder cells.

In some embodiments, the feeder cells are selected from mouse embryonic fibroblasts or STO cells.

In some embodiments, the feeder cells are mouse embryonic fibroblasts.

In some embodiments, the feeder cells are mouse embryonic fibroblasts with cell division arrest.

In some embodiments, the feeder cells are mouse embryonic fibroblasts treated with mitomycin C.

In some embodiments, the feeder cells have a density of 3×10⁴ cells/cm² to 10×10⁵ cells/cm².

In some embodiments, the feeder cells have a density of 5×10⁴ cells/cm².

In some embodiments, the method is performed at a temperature of 37-39° C. (preferably 38.5° C.), oxygen concentration of 5%-22% (preferably 20%), and carbon dioxide concentration of 4%-6% (preferably 5%).

In some embodiments, the medium is refreshed every 10-48 hours (preferably 10-24 hours, more preferably 12 hours) in the method.

### BRIEF DESCRIPTION OF DRAWINGS

FIGS. 1-1A to 1-1G. Lineage separation and tracking of pluripotency changes during embryonic development

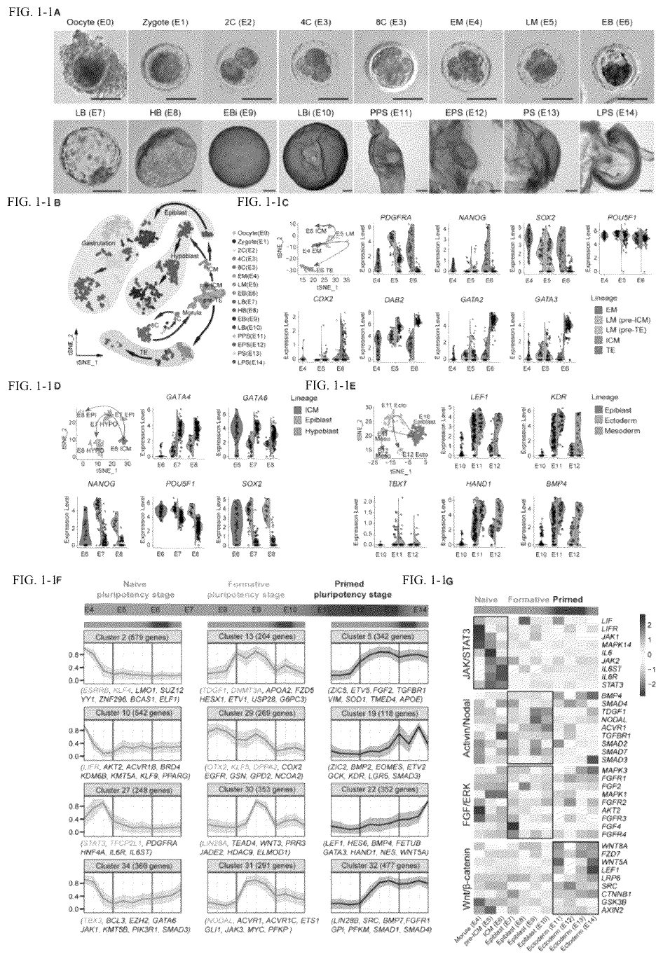

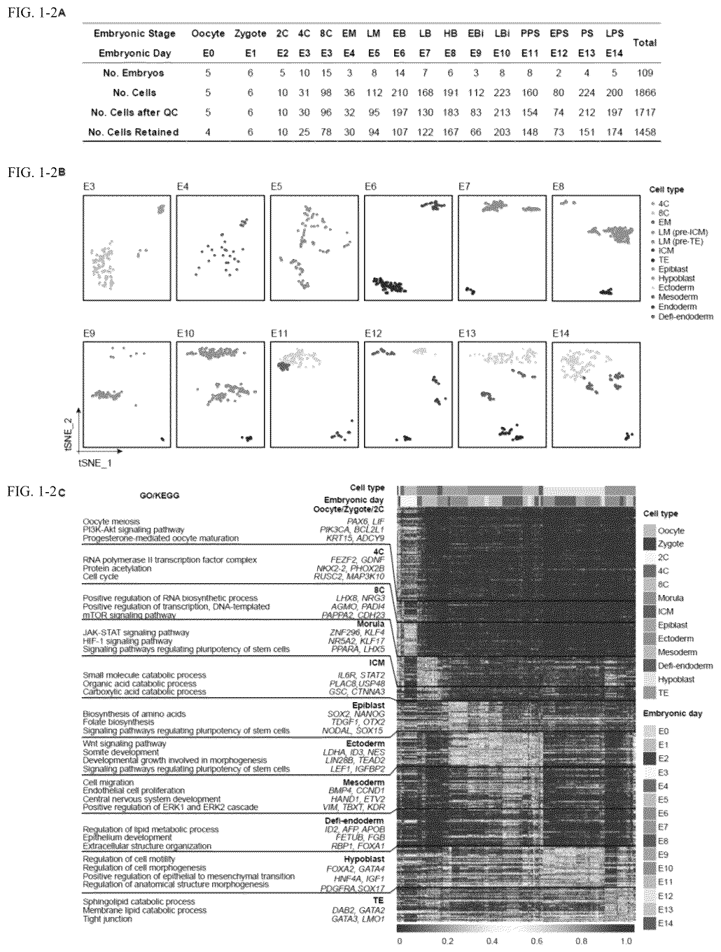

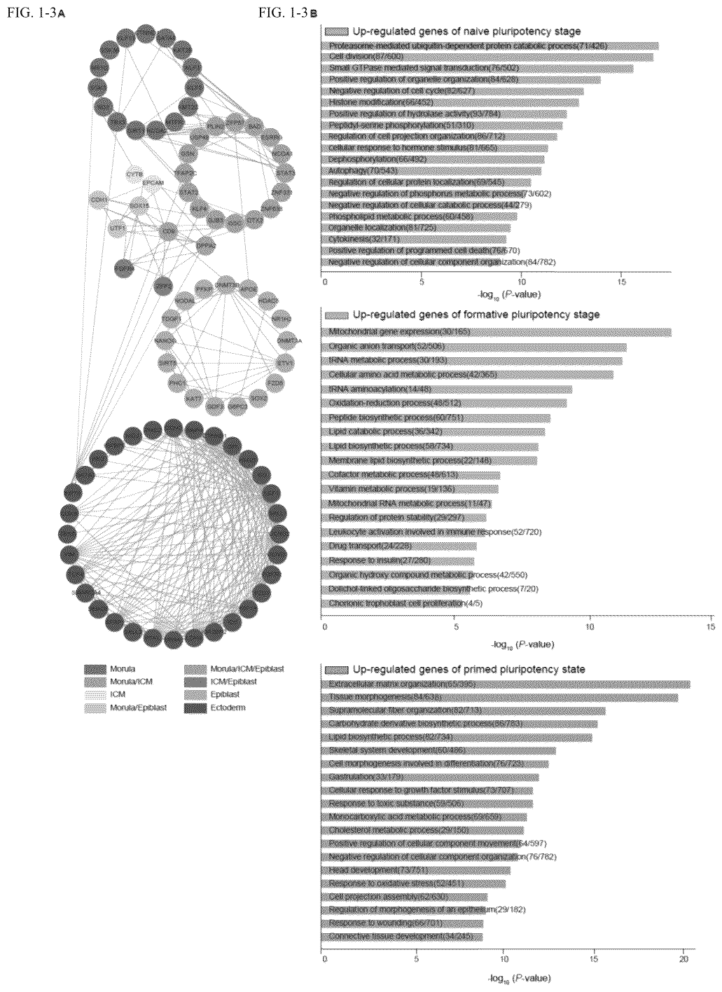

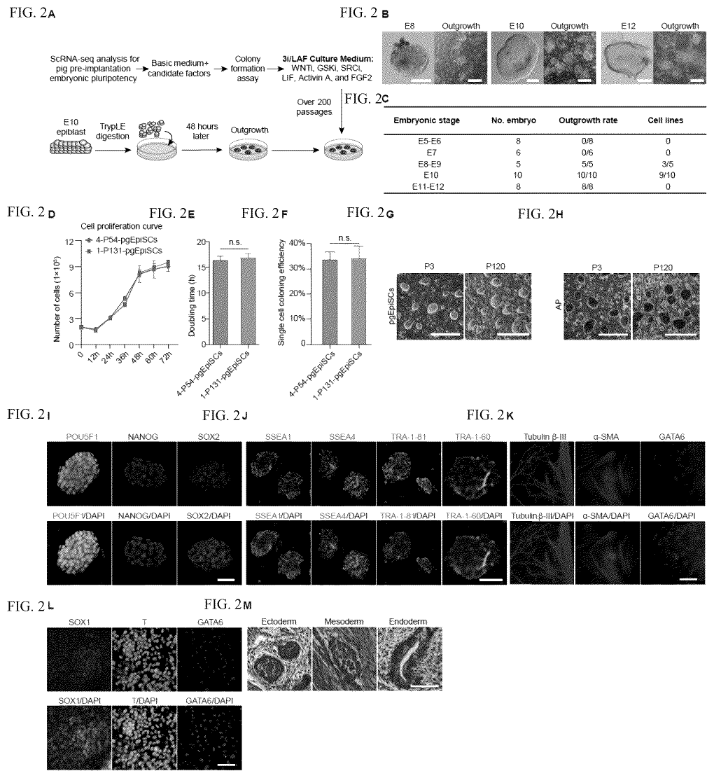

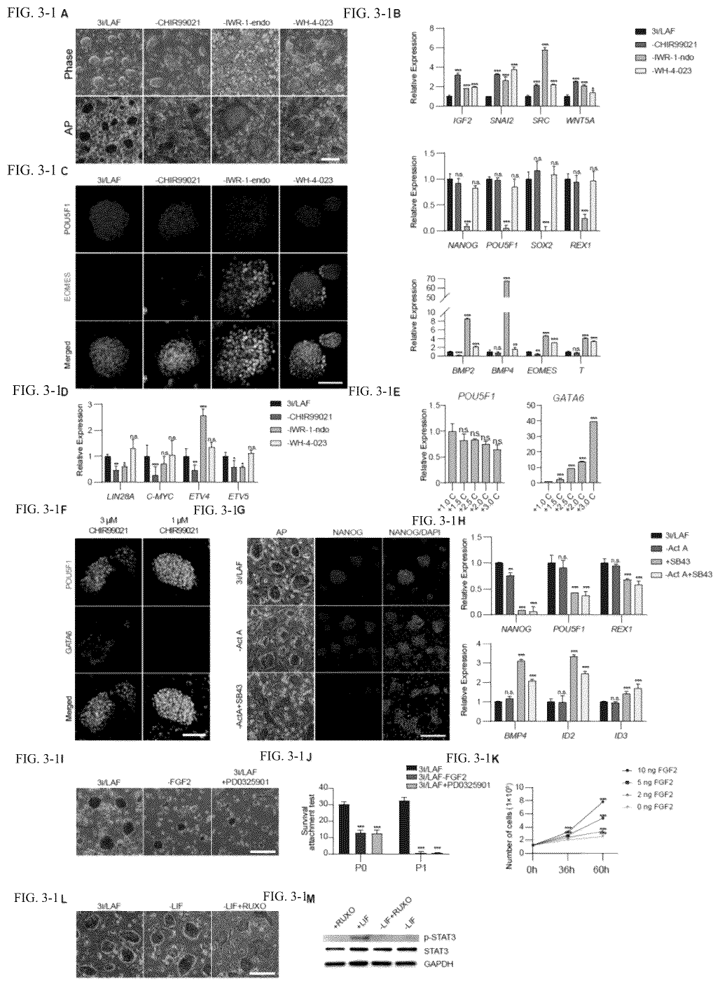

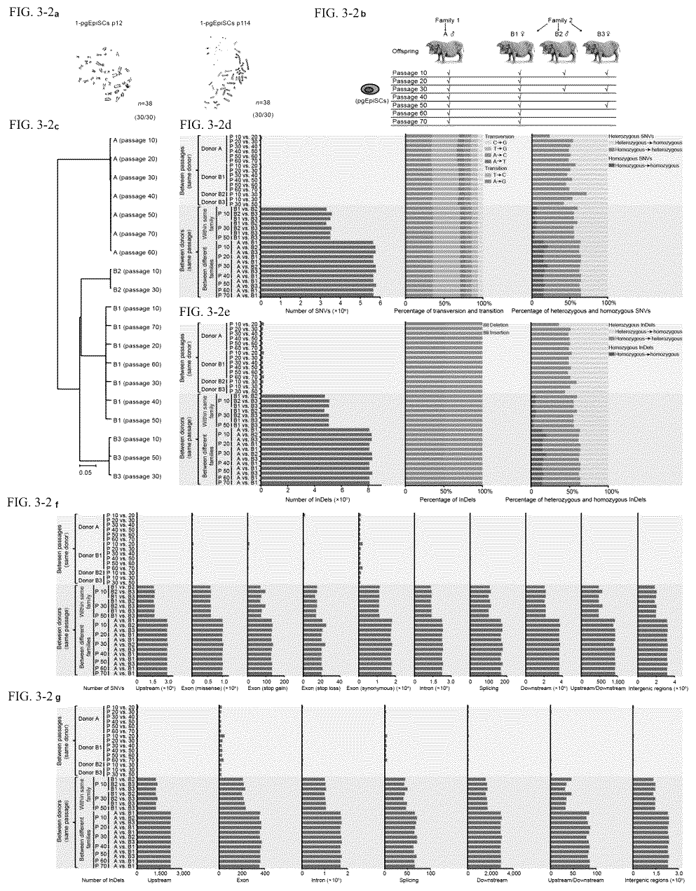

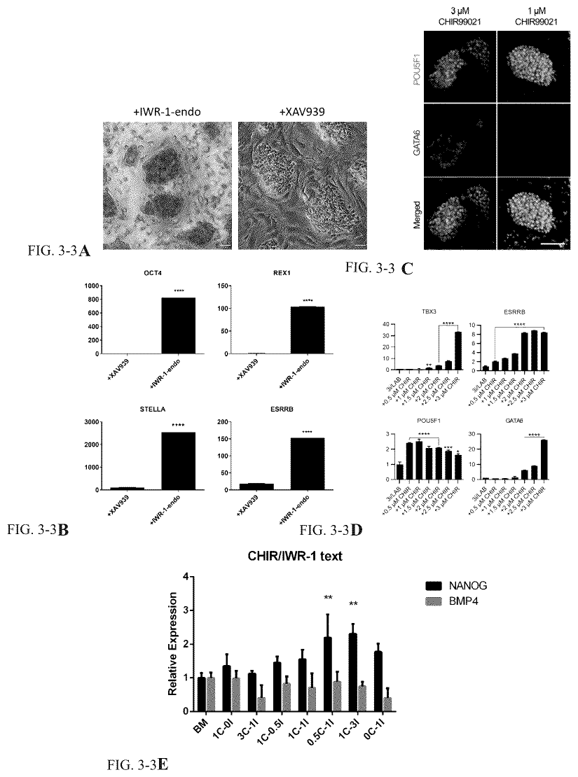

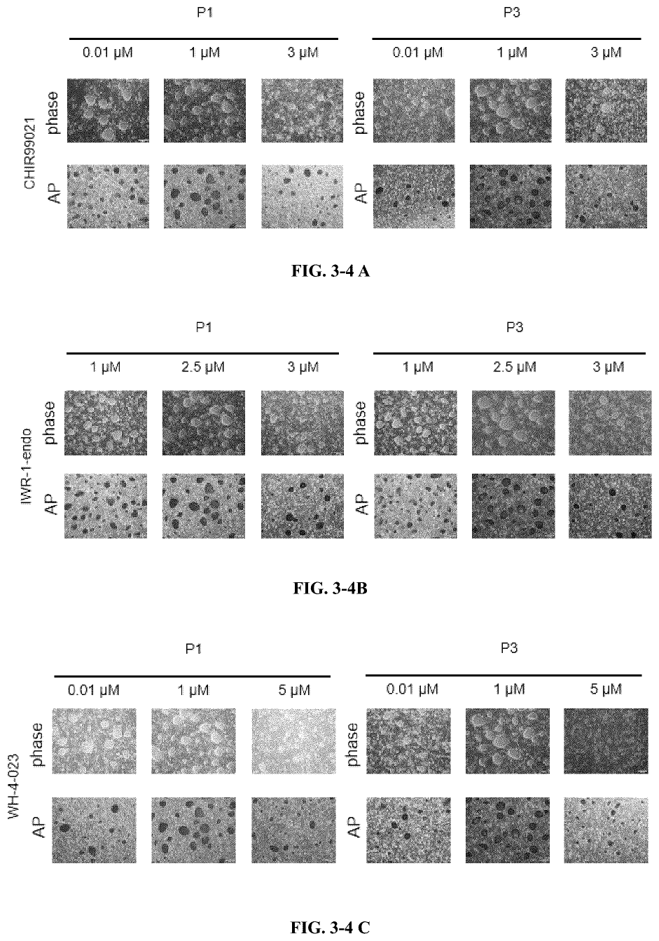

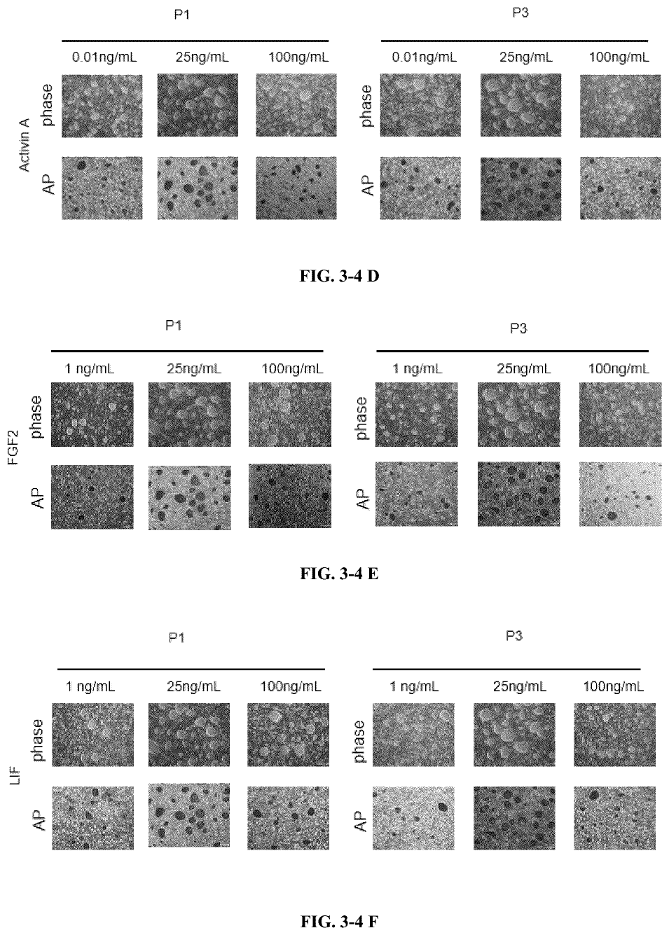

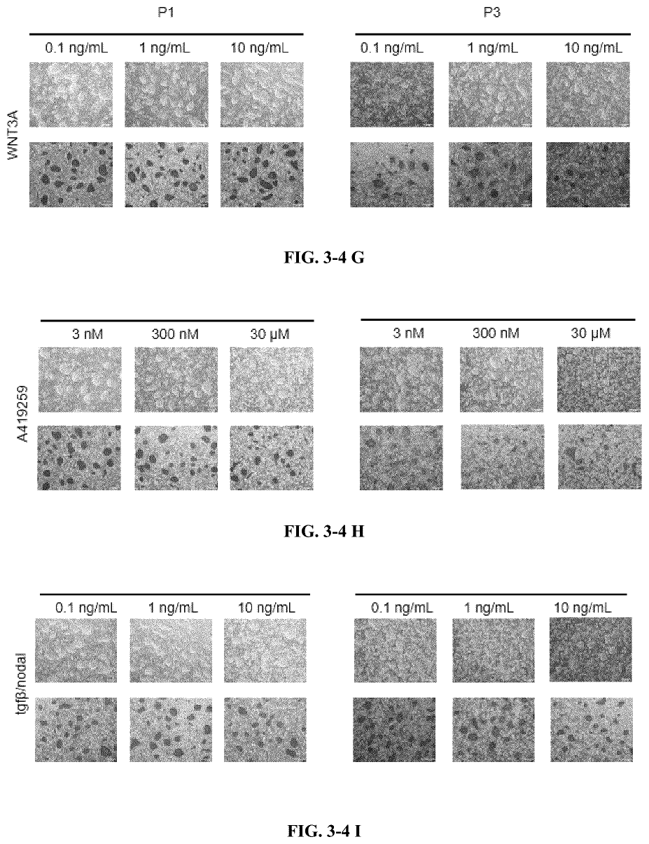

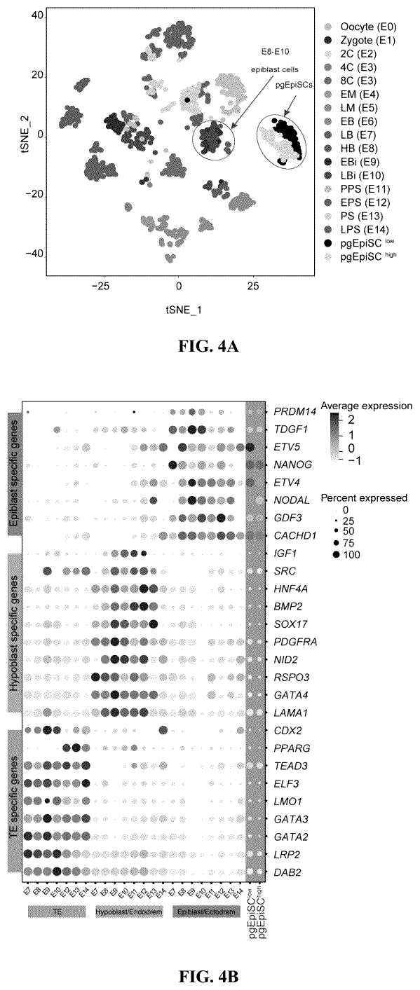

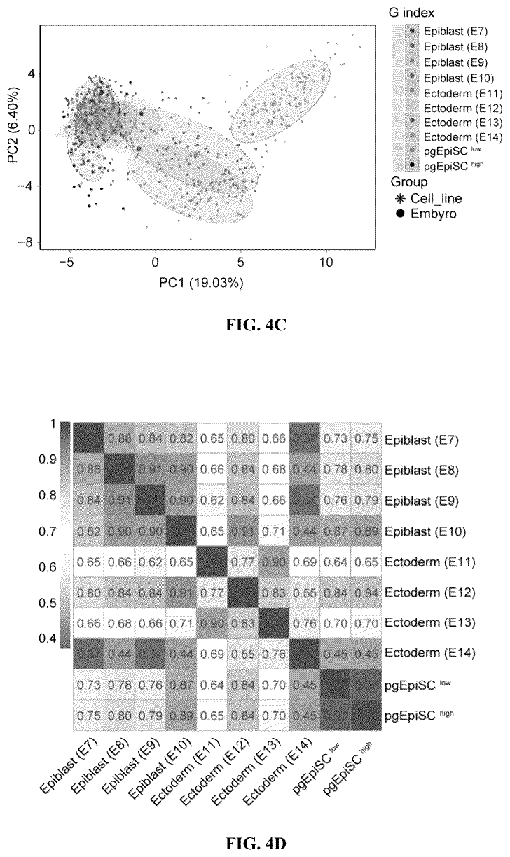

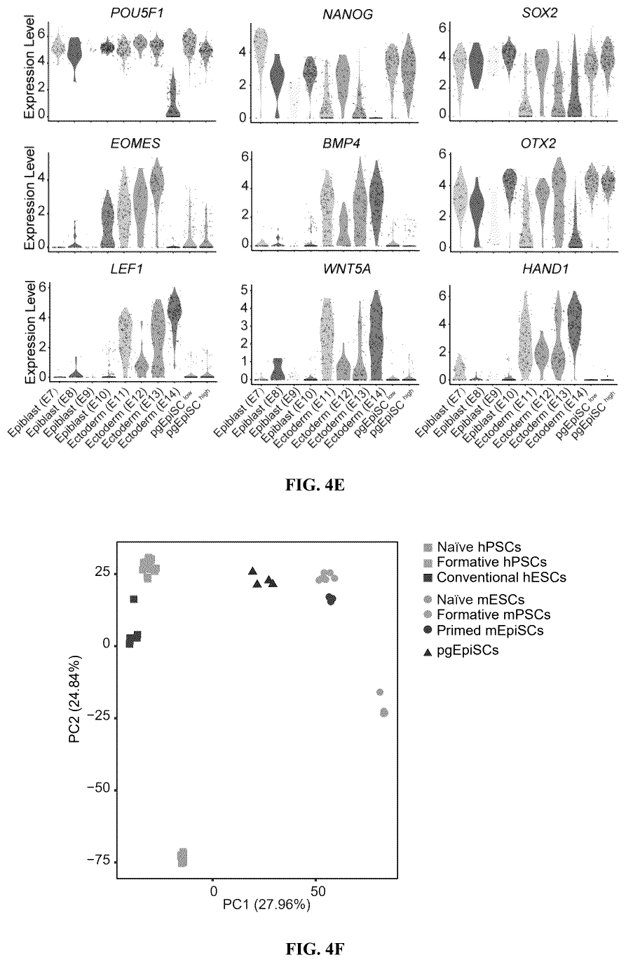

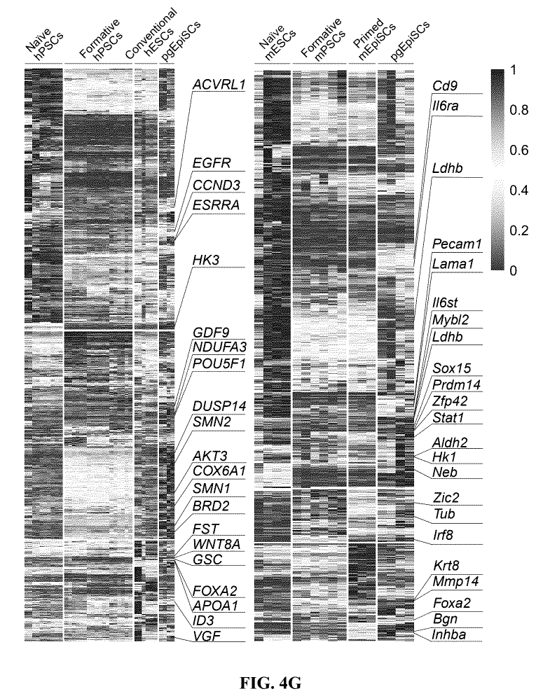

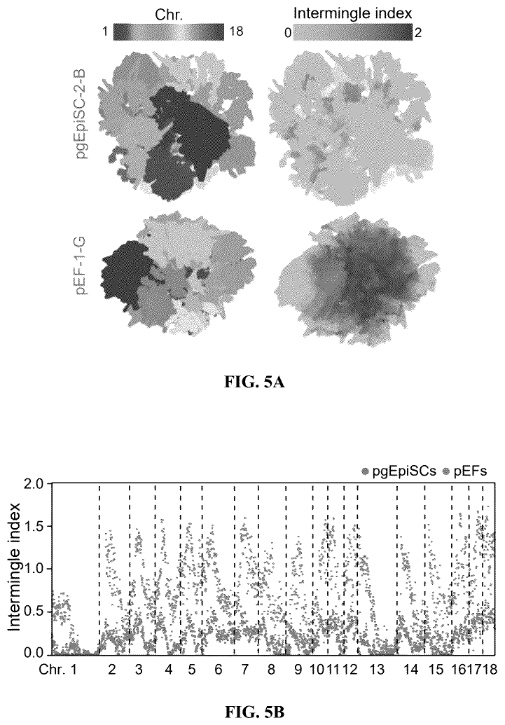

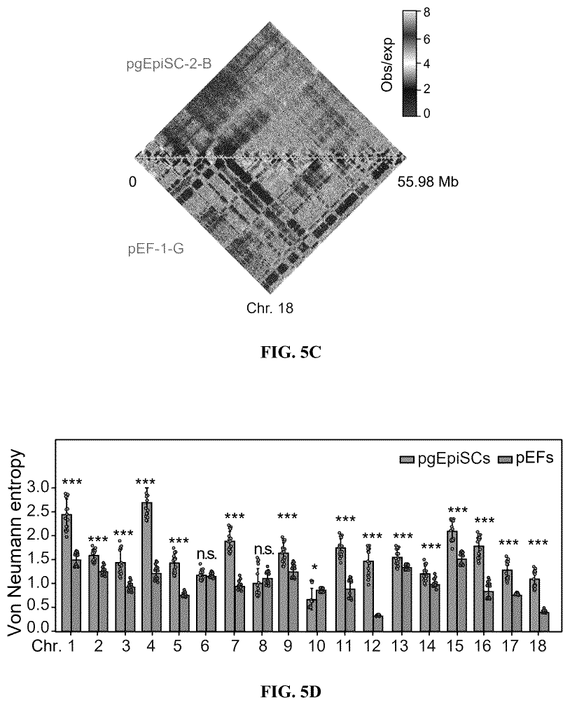

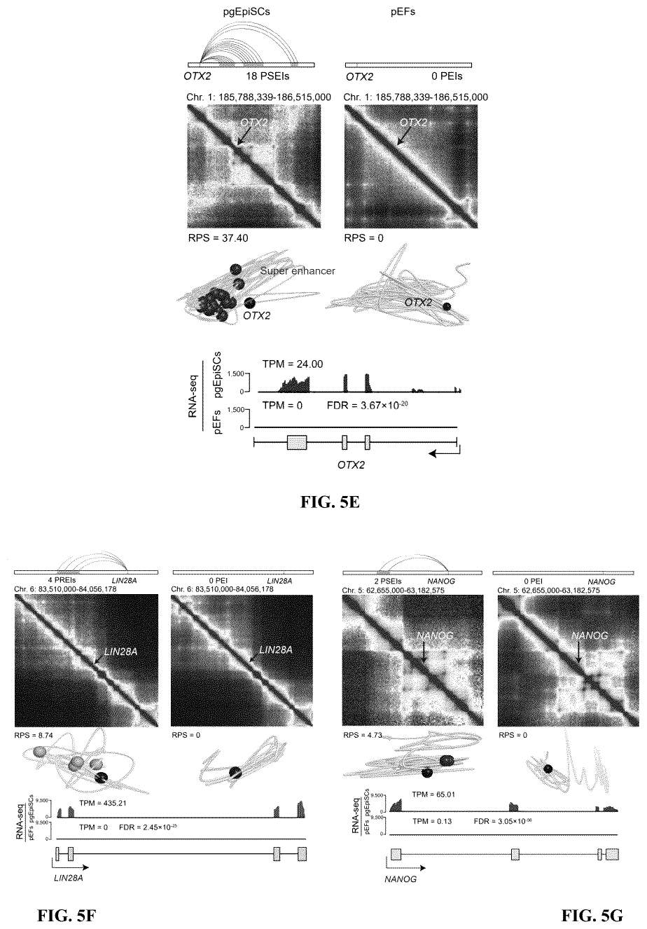

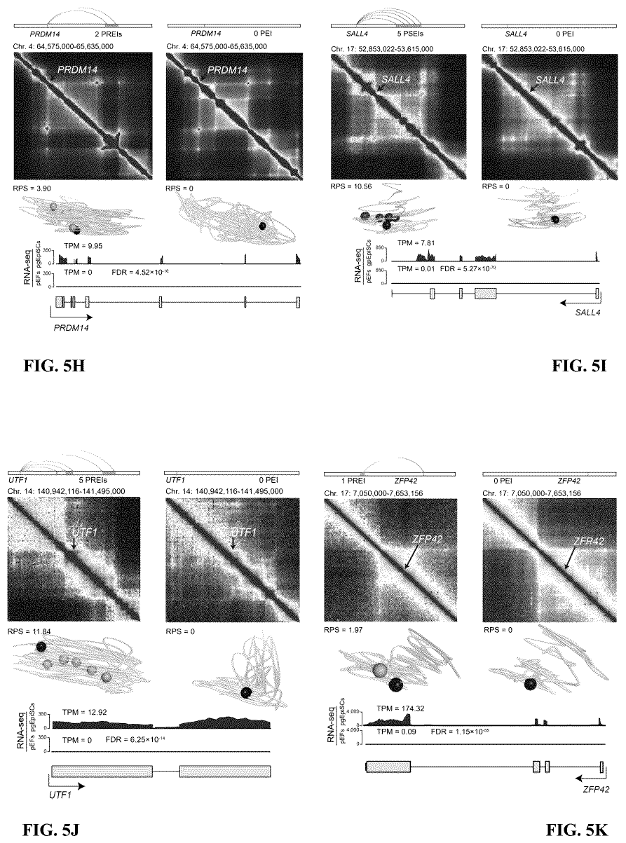

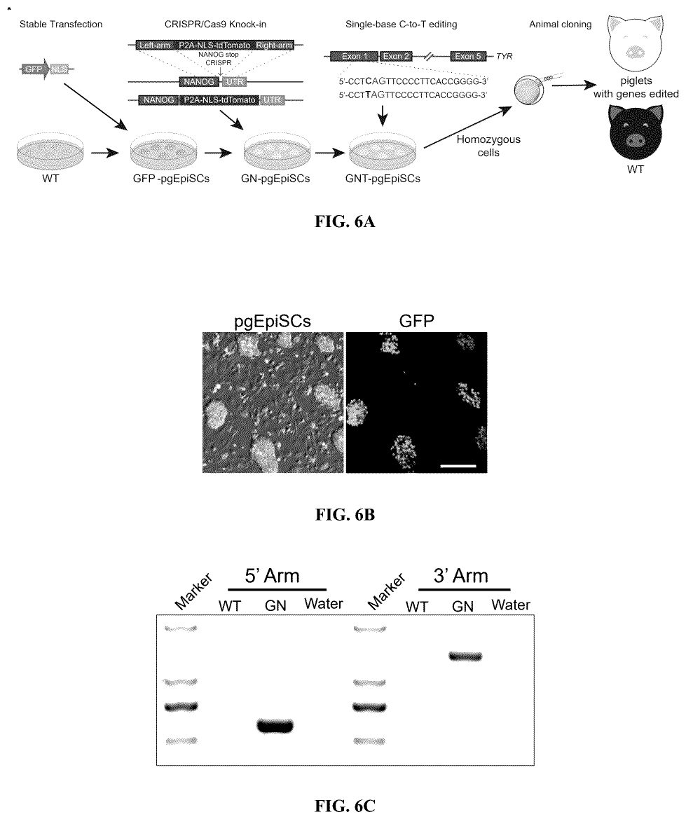

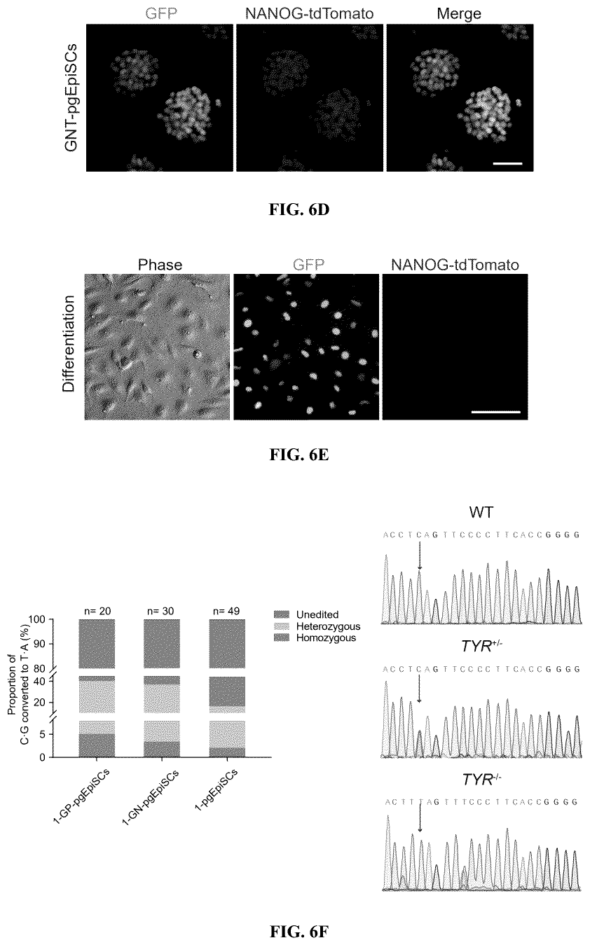

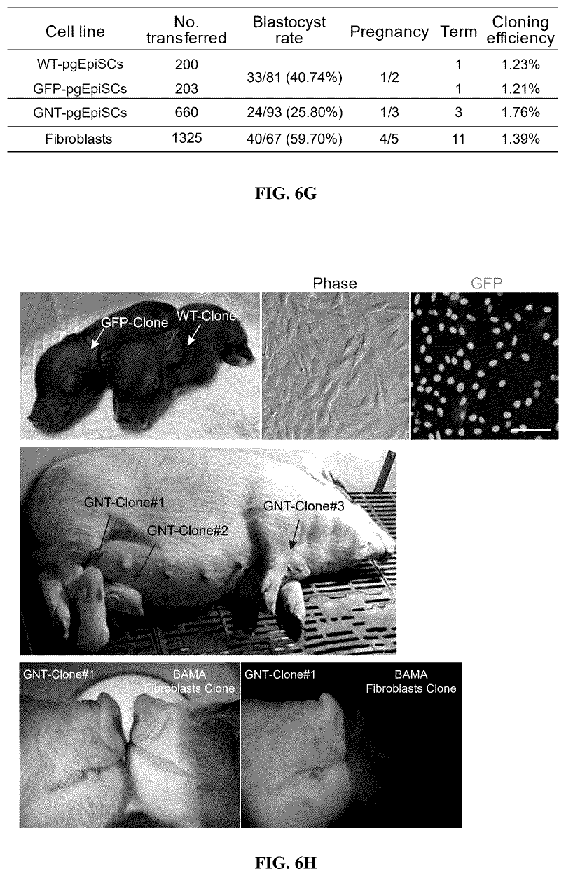

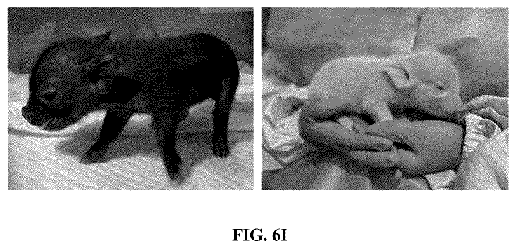

(FIG. 1-1A) Morphological analysis of porcine embryos collected at Embryonic day (E) 0 to E14 for sc-RNA sequence analysis. There are a total of 16 developmental stages, including oocyte (E0), zygote (E1), 2C (2-cell stage embryo, E2), 4C (4-cell stage embryo, E3), 8C (8-cell stage embryo, E3), EM (early morula, E4), LM (late morula, E5), EB (early blastula, E6), LB (late blastula, E7), HB (hatched blastula, E8) EBi (early bilaminar embryo, E9), LBi (late bilaminar embryo, E10), PPS (pre-primitive streak embryo, E11), EPS (early primitive streak embryo, E12), PS (primitive streak embryo, E13), and LPS (late primitive streak embryo, E14) stages. Scale for E0-E8, 100 μm; scale for E9-E14, 500 μm.

(FIG. 1-1B) t-SNE plot shows the transcriptional similarity among all porcine embryonic cells, with dots in different colors representing embryonic days and developmental stages, background colors representing indicated lineages, and arrows indicating known developmental trajectories.

(FIG. 1-1C) t-SNE plot of E4 EM, E5 LM, and E6 EB stage embryonic cells, with arrows indicating ICM/TE lineage separation; violin plots show main lineage separation marker genes.

(FIG. 1-1D) Similar to C, corresponding to E6 EB, E7 LB, and E8 HB embryonic cells, with arrow trajectories indicating epiblast/hypoblast lineage separation.

(FIG. 1-1E) Similar to C, corresponding to embryonic cells in E10 LBi, E11 PPS, and E12 EPS stages, with arrow trajectories indicating ectoderm/mesoderm lineage separation.

(FIG. 1-1F) Representative gene clusters with similar expression trends show changes in naïve, formative, and primed pluripotency related genes in morula (EM and pre-ICM), ICM, epiblast, and ectoderm cells during E4 to E14. The gene names shown in green, yellow, and red represent possible naïve, formative, and primed pluripotency related genes, respectively (Kinoshita et al., 2021; Yu et al., 2021), while the remaining genes (names shown in black) are predicted in the clusters.

(FIG. 1-1G) Heatmaps of expression changes of genes related to JAK/STAT3, Activin/Nodal, FGF/ERK, and Wnt/β-catenin signaling pathway from morula (EM and pre-ICM), ICM, epiblast, and ectoderm cells during E4 to E14, with a gradient from blue to red on the right indicating the transition from low expression to high expression of genes, respectively. The gradient from green to red at the top of the heatmap indicates changes in naïve, formative, and primed pluripotent states.

FIGS. 1-2A to 1-2C. Lineage separation and tracking of pluripotency changes during embryonic development

(FIG. 1-2A) Summary of information on single cell transcriptome sequencing samples. Porcine embryos collected from Embryonic day (E) 0 to E14 are used for sc-RNA sequence analysis. There are a total of 16 developmental stages, including oocyte (E0), zygote (E1), 2C (2-cell stage embryo, E2), 4C (4-cell stage embryo, E3), 8C (8-cell stage embryo, E3), EM (early morula, E4), LM (late morula, E5), EB (early blastula, E6), LB (late blastula, E7), HB (hatched blastula, E8) EBi (early bilaminar embryo, E9), LBi (late bilaminar embryo, E10), PPS (pre-primitive streak embryo, E11), EPS (early primitive streak embryo, E12), PS (primitive streak embryo, E13), LPS (late primitive streak embryo, E14) stages.

(FIG. 1-2B) t-SNE plots of each stage of E3-E14 embryos. Dots in different colors show the lineage separation.

(FIG. 1-2C) A heatmap shows the lineage specific gene expression. In each cell type, genes are ranked by Unsupervised Hierarchical Clustering (UHC). Representative functional categories with relevant genes enrichment are given.

FIGS. 1-3A and 1-3B. Lineage separation and tracking of pluripotency changes during embryonic development

(FIG. 1-3A) Differentially expressed genes (DEGs) network for each cell type compared to others. Different colors represent different cell types. The circle represents the set of DEGs. Each cell type is connected to its DEGs through lines within the network.

(FIG. 1-3B) GO functional enrichment analysis of 1,735 naïve pluripotency related genes, 1,117 formative pluripotency related genes, and 1,289 primed pluripotency related genes in FIG. 3-2 is presented. Summary of the top 20 functional items in the gene set by Metascape (https://metascape.org) is showed, with the number after each item representing the hit gene in the total genes for that item.

FIGS. 2A-2M. Separation and characterization of pgEpiSCs

(FIG. 2A) A strategy for the establishment of stable pgEpiSCs.

(FIG. 2B) Morphological comparison of derivatives from epiblast and ectoderm cells from embryos atdifferent embryonic stages. The embryonic disc (left) and derivatives (right) from E8, E10, E12 are shown sequentially from left to right. Epiblast cells from E8 and E10 exhibit spherical growth, while ectodermal cells from E12 exhibit flat and irregular growth. Scale bars of embryonic discs from E8 and E10, 100 μm; scale bar of embryonic disc from E12, 400 μm; and scale bars of all extensions, 200 μm.

(FIG. 2C) Growth efficiency and cell lines of outgrowth cells at different embryonic stages established in 3i/LAF medium.

(FIG. 2D) Cell proliferation curve of pgEpiSCs. The initial cells are counted at 2×10⁵.

(FIG. 2E) Doubling time of pgEpiSCs.

(FIG. 2F) Efficiency of single cell cloning of pgEpiSCs.

(FIG. 2G) Morphologs of pgEpiSC clones from low- and high-passages. Scale bars, 200 μm.

(FIG. 2H) Detection of low- and high-passage pgEpiSC clones by alkaline phosphatase (AP) staining. Scale bars, 200 μm.

(FIG. 2I) Immunostaining of pluripotency markers POU5F1, NANOG, and SOX2 in pgEpiSCs. Cell nuclei are stained with DAPI. Scale bar, 50 μm.

(FIG. 2J) Immunostaining of pluripotency surface markers SSEA1, SSEA4, TRA-1-81, and TRA-1-60 in pgEpiSCs. Cell nuclei are stained with DAPI. Scale bar, 50 μm.

(FIG. 2K) EB differentiation experiment in vitro. Immunostaining of ectodermal nerve specific marker Tubulin β-III, mesodermal muscle specific marker α-SMA and endodermal specific marker GATA6. Cell nuclei are stained with DAPI. Scale bar, 50 μm.

(FIG. 2L) Immunostaining of pgEpiSCs after directionally induced differentiation. SOX1 represents a neuroectodermal marker, T represents a mesodermal marker, GATA6 represents an endodermal marker, and cell nuclei are labelled with DAPI. Scale bar, 200 μm.

(FIG. 2M) Teratoma formation experiment in vivo. Teratomas derived from pgEpiSCs are stained with hematoxylin and eosin. Scale bar, 100 μm.

Error bars indicated±SD (n=3 independent experiments). For (G-M), similar results are obtained in three independent experiments.

FIGS. 3-1A to 3-1M. Requirement of 3i/LAF culture condition for pgEpiSC maintenance in vitro

(FIG. 3-1A) Comparison of morphology and AP staining of pgEpiSCs without CHIR99021, IWR-1-endo, and WH-4-023. Scale bar, 200 μm.

(FIG. 3-1B) Quantitative analysis of mRNA expression of representative pluripotency marker genes in EMT (top), pluripotency (middle) and mesodermal differentiation (bottom) using qRT-PCR.

(FIG. 3-1C) Immunostaining of the pluripotency marker gene POU5F1 and the mesodermal/endodermal progenitor marker EOMES. Cell nuclei are stained with DAPI. Scale bar, 100 μm.

(FIG. 3-1D) mRNA expression of proliferation related genes quantified by qRT PCR.

(FIG. 3-1E) Relative expression changes of POU5F1 and GATA6 in pgEpiSCs treated with different concentrations of CHIR99021.

(FIG. 3-1F) Immunostaining of POU5F1 and GATA6 in pgEpiSCs treated with different concentrations of CHIR99021. Cell nuclei are stained with DAPI. Scale bar, 100 μm.

(FIG. 3-1G) AP staining and immunostaining of pgEpiSCs cultured without Activin A (Act A) or with SB431542 (SB43), compared with those cultured in 3i/LAF in a control group. Scale bar, 200 μm.

(FIG. 3-1H) Quantitative analysis of mRNA expression of pluripotency and BMP4 signaling pathway related genes using media without Act A and/or supplemented with SB43.

(FIG. 3-11) AP staining test of pgEpiSCs cultured with or without FGF2 or with ERK inhibitor PD0325901. Scale bar, 200 μm.

(FIG. 3-1J) Cell survival and attachment tests of pgEpiSCs in the presence of indicator molecules.

(FIG. 3-1K) Cell proliferation curves of pgEpiSCs treated with different concentrations of FGF2.

(FIG. 3-1L) AP staining detection of pgEpiSCs cultured with LIF or with the addition of JAK1/2 inhibitor ruxolitinib (RUXO). Scale bar, 200 μm.

(FIG. 3-1M) Function of LIF observed by Western blot in the maintenance of pgEpiSCs in vitro.

For (B), (D), (E), (H), (J), and (K) error bars indicate ±SD (n=3 independent experiments), n.s., P≥0.05; *, P<0.05; **, P<0.01, ***, P<0.001. For (A), (C), (F), (G), (I), (L), and (M), similar results were obtained in three independent experiments.

FIGS. 3-2a to 3-2g. Characteristics of SNVs and Short InDels (≤30 bp) in pgEpiSC cell lines from different passages in the same cell line and between different cell lines.

(FIG. 3-2a) Karyotype analysis of pgEpiSCs from low- and high-passages. For each cell line, 30 cells at the mitosis metaphase are detected.

(FIG. 3-2b) Schematic illustration showing whole genome resequencing for 19 pgEpiSC samples (each with a sequencing depth of 24.42×) from 4 independent donor cell lines (A, B1, B2, and B3). It is noted that the three independent donor cell lines (i.e. B1, B2, and B3) are full siblings.

(FIG. 3-2c) Neighbor-joining (NJ) phylogenetic tree of 19 pgEpiSC cell lines. Scale bar represents the p distance.

(FIG. 3-2d, FIG. 3-2e) Numbers and compositions of SNVs (FIG. 3-2d) and InDels (FIG. 3-2e). In contrast, there are a large number of gene mutations between pgEpiSCs from the same passage but different donors (between three full siblings: ˜3.46 M SNVs [Ts/Tv ratio: ˜2.41] and ˜498.10 K InDels; between different families: ˜5.71 M SNVs (Ts/Tv ratio: ˜2.41) and ˜816.72 K InDels). The number of mutations (˜37.98 K SNVs [Ts/Tv ratio: ˜2.13] and ˜15.58 K InDels) in pgEpiSCs from the same donor after multiple passages is only a small proportion (SNVs: ˜1.10%; InDels: ˜3.13% compared with two full siblings). In addition, compared with the proportion of homozygous mutations in pgEpiSCs from the same donor after multiple passages (SNVs: ˜0.08%; InDels: ˜0.22%), there is a significant increase in homozygous mutations between pgEpiSCs from different donors (between three full siblings: SNVs: ˜5.22%, InDels: ˜4.96%; SNVs and InDels between different family donors are ˜16.67% and ˜16.02%, respectively).

(FIG. 3-2f, FIG. 3-2g) Summary and annotation of SNVs (FIG. 3-2f) and InDels (FIG. 3-2g) in different genomic elements. Each SNV and InDel gene locus is annotated using the ANNOVAR software package.

FIGS. 3-3A to 3-3E. Effects of different culture conditions on the preparation of pgEpiSCs.

(FIG. 3-3A) Effect of the replacement of IWR-1-endo with XAV939 on maintaining cell pluripotency, with AP positivity lost after around 8 passages.

(FIG. 3-3B) Effect of the replacement of IWR-1-endo with XAV939 on the expressions of core pluripotency factor OCT4 (POU5F1), pluripotency factors REX1, STELLA, and ESRRB.

(FIG. 3-3C) Effects of different concentrations of CHIR99021 on the expressions of pluripotency gene POU5F1 and lineage differentiation gene GATA6.

(FIG. 3-3D) Effects of different concentrations of CHIR99021 on the expressions of pluripotency gene POU5F1, lineage differentiation gene GATA6, mesodermal marker gene TBX3, and endodermal gene ESRRB.

(FIG. 3-3E) Effect of the concentration ratio of CHIR99021 to IWR-1-endo on the expression of the pluripotency factor Nanog.

FIG. 3-4A. Effect of concentration adjustment of CHIR99021 in 3i/LAF medium on the pluripotency of pgEpiSCs in vitro. The upper panel indicates before AP staining, and the lower panel indicates after AP staining.

FIG. 3-4B. Effect of concentration adjustment of IWR-1-endo in 3i/LAF medium on the pluripotency of pgEpiSCs in vitro. The upper panel indicates before AP staining, and the lower panel indicates after AP staining.

FIG. 3-4C. Effect of concentration adjustment of WH-4-023 in 3i/LAF medium on the pluripotency of pgEpiSCs in vitro. The upper panel indicates before AP staining, and the lower panel indicates after AP staining.

FIG. 3-4D. Effect of concentration adjustment of recombinant human ActivinA in 3i/LAF medium on the pluripotency of pgEpiSCs in vitro. The upper panel indicates before AP staining, and the lower panel indicates after AP staining.

FIG. 3-4E. Effect of concentration adjustment of recombinant human FGF-basic in 3i/LAF medium on the pluripotency of pgEpiSCs in vitro. The upper panel indicates before AP staining, and the lower panel indicates after AP staining.

FIG. 3-4F. Effect of concentration adjustment of recombinant human LIF in 3i/LAF medium on the pluripotency of pgEpiSCs in vitro. The upper panel indicates before AP staining, and the lower panel indicates after AP staining.

FIG. 3-4G. Effect of replacing CHIR99021 with WNT3a in 3i/LAF medium on the pluripotency of pgEpiSCs in vitro. The upper panel indicates before AP staining, and the lower panel indicates after AP staining.

FIG. 3-4H. Effect of replacing WH-4-023 with A419259 in 3i/LAF medium on the pluripotency of pgEpiSCs in vitro. The upper panel indicates before AP staining, and the lower panel indicates after AP staining.

FIG. 3-41. Effect of replacing recombinant human Activin A with Nodal in 3i/LAF medium on the pluripotency of pgEpiSCs in vitro. The upper panel indicates before AP staining, and the lower panel indicates after AP staining. FIG. 4A: t-SNE map plotted using scRNA-seq data from pre-implantation porcine embryonic cells (n=1458) and pgEpiSCs (n=196). Clusters are color-coded based on the embryo age and the passage of pgEpiSCs. The circled areas represent pre-gastrulation epiblast cells and pgEpiSCs.

FIG. 4B: Dot plot of classical marker genes for TE, hypoderm, and epiblast during porcine embryonic development. The color gradient represents the average expression level, and the size of the dots corresponds to the percentage of cells expressing characteristic genes in TE, hypoderm and epiblast cell populations.

FIG. 4C: PCA plot of pgEpiSCs and epiblast or ectoderm cells from E7 to E14. Each dot represents a single cell in pre-implantation embryonic cells, and the asterisk represents a single cell in pgEpiSCs. Colors indicate the embryo age and the passage of pgEpiSCs.

FIG. 4D: Spearman correlation coefficient is based on the average expression level of specific expressed genes in various epiblast or ectoderm cells from E7 to E14, which is related to the regulation of pluripotency and epithelial cell differentiation.

FIG. 4E: Violin plot showing the expression levels (log 2 (TPM/10+1)) of classical pluripotency related genes in pgEpiSCs from E7 to E14 and low- and high-passages based on scRNA-seq data. Each dot represents a single cell.

FIG. 4F: A plot of PCA results for naïve, formative, and conventional hPSCs; naïve, formative, and primed mPSCs; and pgEpiSCs, which is based on a set of uniquely expressed genes for each type of PSC. Colors represent the state of pluripotency. Triangles represent pigs, squares represent humans, and circles represent the murine.

FIG. 4G: Comparison of the expression levels of uniquely expressed genes identified in naïve hPSCs, formative hPSCs, and conventional hESCs (left) and naïve mESCs, formative mPSCs, and primed mEpiSCs (right) with the expression levels of these genes in pgEpiSCs. The listed genes are highly expressed in pgEpiSCs.

FIG. 5A: Hi-C maps with their respective resolutions of 100 kb for intrachromosome and 1 mb for interchromosome, respectively. Cross-sectional examples of pgEpiSCs-1-B and pEF-1-G nuclei colored according to the autosome (left) or by the multi-chromosome intermingle index (reflecting chromosomal diversity measured by Shannon's index) (right) (Tan et al., 2018).

FIG. 5B: Probability of broad multi-chromosome intermingle of 18 autosomes (smoothed by a 1 mb window) in pgEpiSCs (green) and pEFs (red) at a resolution of 100 kb (average of 16 Hi-C maps for each cell type) (Tan et al., 2018).

FIG. 5C: Example of the contact map of chromosome 18 in pgEpiSCs-1-B (top half) and pEF-1-G (bottom half) at a resolution of 100 kb.

FIG. 5D: The extent of disorder in chromatin structure (quantified by Von Neumann entropy) (Lindsly et al., 2021) in pgEpiSCs (green) and pEFs (red) at 100 kb resolution in each of 16 Hi-C maps.

FIGS. 5E-5K: PEIs schematics for OTX2 (E), LIN28A (F), NANOG (G), PRDM14 (H), SALL4 (I), UTF1 (J), and ZFP42 (K). Top panel: Hi-C map of the regions near the center of the gene transcription start site (+250 kb). Middle panel: Three-dimensional models of the promoter (blue sphere) and its enhancer (red and green spheres represent the super enhancer and regular enhancer, respectively). Bottom panel: RNA-seq profile. Benjamin Hochberg-adjusted FDR is calculated.

FIG. 6A: Schematic illustration for cloned piglets generated by a multi-gene continuous editing strategy in the nuclear transfer experiment using pgEpiSCs as the donor.

FIG. 6B: Morphology and fluorescence detection of GFP-pgEpiSC clones, with a scale bar of 200 μm.

FIG. 6C: NANOG-tdTomato knock-in identified by PCR. “GN” represents GFP positive pgEpiSCs with NANOG-tdTomato knock-in.

FIG. 6D: Expression of NANOG-tdTomato knock-in reporter genes in GFP labeled pgEpiSCs. Scale bar, 100 μm.

FIG. 6E: Expression loss of tdTomato in pgEpiSCs knocked-in by the NANOG-tdTomato reporter gene after differentiation. Scale bar, 200 μm.

FIG. 6F: Statistics on the unedited ratio, homozygous ratio, and heterozygous ratio of mutations from C to T of TYR gene in WT pgEpiSCs (1-pgEpiSCs) and genetically modified pgEpiSCs (1-GP-pgEpiSCs and 1-GN-pgEpiSCs) (left); and representative DNA sequencing analysis of the sites of mutations from C to T of TYR gene in wild-type, heterozygous, and homozygous pgEpiSCs (right).

FIG. 6G: Summary of the nuclear transfer experiment of pgEpiSCs. The blastocyst rate is calculated using embryos retained before transplantation. Fibroblasts are derived from the BAMA pig.

FIG. 6H: Top panel: ear fibroblasts from WT pgEpiSCs cloned piglets and GFP-pgEpiSCs cloned piglets; Middle panel: three GNT-pgEpiSCs cloned piglets and their surrogate mother; Bottom panel: representative GNT-pgEpiSCs cloned piglets displaying GFP fluorescence by contrast to WT piglets cloned from fibroblasts in the Bama pig.

FIG. 6I: Representative cloned piglets produced from WT pgEpiSCs as donor cells (left) exhibiting black fur color, while representative cloned piglets produced from GNT-pgEpiSCs as donor cells (right) exhibiting a phenotype of white fur color albinism.

### DETAILED DESCRIPTION OF EMBODIMENTS

Examples of the present invention described in detail below are illustrative and are intended to interpret the present invention, but are not to be construed as limitations of the present invention.

Since the 1990s, researchers have been attempting to establish a stable stem cell line derived from porcine epithelial cells, but have never been successful. The inventors develop a serum-free medium in vitro for the establishment and maintenance of stable pre-gastrulation epiblast stem cell lines (pgEpiSCs) from porcine embryos at E10 by the large-scale single cell transcriptome analysis of porcine embryos at day 0 to 14. PgEpiSCs maintain pluripotency and normal karyotype over more than 200 passages by chemically inhibiting wnt related signaling pathways during culture. Interestingly, the high-deep Hi-C in situ analysis reveals the functional effect of three-dimensional spatial correlation on the transcriptional regulation of pluripotency marker genes in pgEpiSCs. In fact, the inventors have confirmed that at least three rounds of continuous gene editing may be tolerated by pgEpiSCs, and live cloned piglets with gene editing have been produced by the somatic cell nucleus transfer technology. The discovery by the inventors provides hope for long-awaited porcine pluripotent stem cells and opens new avenues for the biological research, animal husbandry and regenerative biomedicine.

The present invention is further explained in connection with specific examples below.

Unless otherwise specified, methods for the molecular biology experiment and immunoassay used in the present invention are generally carried out in accordance with those described in J. Sambrook et al., Molecular Cloning: A Laboratory Manual, 2nd Edition, Cold Spring Harbor Laboratory Press, 1989, and F. M. Ausubel et al., Short Protocols in Molecular Biology, 3rd Edition, John Wiley & Sons, Inc., 1995; and restriction endonucleases are used under the conditions recommended by the product manufacturer. A person skilled in the art should understand that the present invention is described by way of example through examples, and it is not intended to limit the scope of protection claimed by the present invention.

The pgEpiSC described in the examples below refers to a cell line established from the epiblast at E10.

### EXPERIMENTAL METHODS

### Preparation and Sequencing of a Single Cell RNA Library

As in previous studies, a single cell RNA-seq library was prepared using a modified Smart-seq2 protocol (Gao et al., 2018; Wang et al., 2018). In brief, a single embryonic cell was transferred into a prepared lysis buffer containing 8 bp bar code. Then, the first strand cDNA was reverse transcribed and amplified in a reverse transcription (RT) mixture containing 4 U RNase inhibitor, 100 U SuperScript II reverse transcriptase (Invitrogen, 18064071), 1 mM dNTPs (TAKARA, 4019), 60 mM MgCl₂, and 3 μM RT primers and 10 μM TSO primers. After PCR amplification, the product was purified with 0.8× AMPure XP beads (Beckman, A63882). Biotin-labeling PCR amplification was then performed. Finally, the single cell RNA-seq library was constructed based on the PCR library Amplification/Illumina series KAPA Hyper Prep kit (KAPA, KK8054). A high-quality library was sequenced at 150 bp at the paired ends on Illumina Hiseq Xten (Novogene).

### Cell Growth Curve and Population Doubling Time

Porcine EpiSCs were cultured in 12-well plates. Cell samples were seeded in triplicate at a density of 2×10⁴ cells per well. Cells were counted every 12 hours. Cells were digested and counted using the Luna™ automated cell counter at each time point. The results of the three counts were averaged and plotted. The cell doubling time was calculated as follows: Doubling Time (DT)=24×[lg2/(lgNₜ−IgN₀)], where 24 was the cell culture time (hours); Nₜ was the number of cells cultured for 48 hours; and No was the number of cells recorded at 24 hours.

### Analysis of Single Cell Cloning Efficiency

Cells were dissociated by Accutase (Gibco, A11105-01), counted using a hemocytometer, and seeded onto the feeder layer cells pre-seeded in 6-well plates in triplicate at a density of 100, 200, and 1,000 cells per well under conditions for pgEpiSCs culture. The clones were counted using AP staining after 6 days, and the efficiency of clone formation was assessed as a percentage of the number of clones per number of cells seeded.

### Karyotype Analysis

Before karyotyping, cells were incubated in the medium for pgEpiSCs with addition of 1% KaryoMAX Colcemid solution (Gibco, 15212012) for 1 hour. PgEpiSCs were digested into single cells by TrypLE™ Express (Gibco, 12605010) and collected by centrifugation. PgEpiSCs were resuspended in a low osmotic solution of 0.075 M KCl (sigma, P5405) and incubated at 37° C. for 15 minutes. PgEpiSCs were then fixed with methanol and acetic acid at a ratio of 3:1, which was repeated for three times. The pgEpiSCs suspension was dropped onto a pre-cooled glass slide, thoroughly dried at room temperature, and then stained with 10% Giemsa staining solution (Sangon, E607314-0001) for 30 minutes. For each cell line, more than 30 cells at the mitosis metaphase were detected.

### Whole Genome Sequencing

The total DNA of pgEpiSCs was extracted using the TIANamp genomic DNA kit (TIANGEN, DP304). After DNA extraction, 1 μg of genomic DNA was randomly fragmented using Covaris, and fragments of 200-400 bp were selected using the Agencourt AMPure XP-Medium kit (BERCKMAN COULTER, A63880). The terminal in the selected fragment was repaired and adenosylated at 3′, followed by linking with an adapter.

The product was amplified by PCR, and then the purified PCR product was thermally denatured into a single strand and cyclized using a splint oligonucleotide sequence. The single-stranded loop DNA was formatted into the final library and validated by quality control. The final validated library was sequenced using BGISEQ-500.

### Alkaline Phosphatase (AP) Staining

The alkaline phosphatase staining for pgEpiSCs was based on the Alkaline Phosphatase Detection Kit (Millipore, SCR004).

The specific experimental steps followed the instructions for the kit.

### Immunofluorescence Assay

Cells were washed with DPBS (Gibco, C14190500BT) and fixed with 4% paraformaldehyde at room temperature for 30 minutes, then washed again with DPBS, permeabilized in 0.1% Triton X-100 for 20 minutes, and blocked with 3% BSA for 1 hour. Cells were incubated with the primary antibody diluted with 3% BSA overnight at 4° C. Cells were then washed 3 times with the washing buffer (DPBS containing 0.1% Triton X-100 and 0.1% Tween 20) for 3 minutes each time. Cells were incubated with diluted secondary antibody and washing buffer together at room temperature for 1 hour, then washed 3 times with the washing buffer for 5 minutes, followed by nuclear staining with DAPI (Roche Life Science, 10236276001) for 3 minutes.

### Embryoid Differentiation

PgEpiSCs were dissociated using Accutase (Gibco, A11105-01) and separated from feeder cells using differential attachment method, followed by culturing in DMEM (Gibco, 11960-044) supplemented with 10% FBS (Gibco, 11960-044), 1% penicillin-streptomycin (Thermo Fisher Scientific, 15140-122) and 1% glutamine (Thermo Fisher Scientific, 35050-061) in a 35 mm low attachment dish that was placed on a horizontal shaker at 50 rpm for 5-7 days. Regular spherical EB was selected and plated on a gelatin coated plate in the same medium within 1 week, and then fixed and detected using the same method as immunofluorescence.

### Induced Directional Differentiation

3i/LAF medium was replaced with neural induction medium I (2.5 μM IWR-1-endo, 5 μM SB431542 and 10 ng/ml FGF2 in BM) two days after pgEpiSCs passage for neural induction. The medium was refreshed with neural induction medium II (4 μM RA, 10 ng/mL FGF2 and 20 ng/mL Noggin in BM) after 2 days of culture, and the immunostaining was performed 2 days later.

31/LAF medium was refreshed with 10 ng/ml BMP4, 5 μM SB431542 and 10 ng/mL FGF2 two days after pgEpiSCs passage for endodermal induction. Immunostaining was performed after passage.

3i/LAF medium was replaced with mesodermal induction medium I (10 ng/mL BMP4, 50 ng/ml Activin A and 20 ng/mL FGF2 in BM) two days after pgEpiSCs passage for mesodermal induction, which was used to culture for two days. Subsequently, mesodermal induction medium II (3 μM IWR-1-endo, 5 μM CHIR99021, 20 ng/ml FGF2 in BM) was added, followed by immunostaining 2 days later.

### Teratoma Formation

About 1×10⁷ dissociated pgEpiSCs cells were collected by centrifugation at 1,000 rpm for 5 minutes, which were then injected subcutaneously into the posterior neck of BALB/c nude mice for analysis of teratoma formation. Teratoma might be observed after 4-5 weeks of feeding.

### H&E Analysis

Teratomas were collected subcutaneously from nude mice, washed twice in PBS, and fixed with 4% PFA at 4° C. for 2 days. Teratoma tissues were dehydrated by alcohol gradients (70%, 80%, 90%, 95%, and 100%) for 1 hour each, then transferred to xylene and embedded in paraffin to obtain samples, which were sectioned into a thickness of 5 μm, followed by dewaxing in xylene and rehydrating with reduced concentration of ethanol. Samples were then stained with hematoxylin (Sigma Aldrich, MHS16) and eosin (Sigma Aldrich, HT110116), and observed under a microscope (Leica, DM5500B).

### RT-qPCR

Total RNA of pgEpiSCs was extracted using a RNAprep Pure Cell/Bacteria Kit (TIANGEN, DP430), and then reverse transcribed into cDNA using 5× all-in-one RT premix (Abm, G490). PCR was performed using 2× RealStar Green Power Mixture (GenStar, A311-05) on the LightCycler 480 II real-time system (Roche). Comparative CT (2^{-ΔΔCT}) method was used to analyze the data. ACT was calculated using EF1A as an internal control. Three biological replicates were performed for all experiments. Primers for quantitative real-time PCR were listed in the key resource table.

### Western Blot

Total proteins in cells were extracted using cell lysis buffer (Beyotime Biotechnology, P0013), and nuclear and cytoplasmic proteins were extracted using a cell nucleus and cytoplasmic protein extraction kit (Beyotime Biotechnology, P0027) supplemented with protease and phosphatase inhibitors (P1050). The concentration of extracted proteins was measured using a Bradford protein detection kit (Bio-red, 5000201). An equal amount of proteins (15 μg) were separated by SDS-PAGE gel electrophoresis, and then transferred from the gel to the Immobilon-P transfer membrane (Merck Millipore; pore size: 0.45 μm; IPVH00010). The blots were blocked in 5% skim milk powder (Sangon Biotech, A600669-0250) in TBST (20 mM Tris, pH 7.5; 150 mM NaCl; 0.1% Tween 20) at room temperature for 1 hour, and then incubated with the antibody diluted in 5% skim milk powder in TBST at 4° C. overnight. The next day, the blots were rinsed 3 times with TBST for 5 minutes each time, and then incubated with HRP conjugated secondary antibody diluted in 5% skim milk powder in TBST at room temperature for 1 hour, and finally rinsed 3 times with TBST for 5 minutes each time. The blots were developed using SuperSignal® West Dura Extended Duration Substrate (Thermo Fisher Scientific, 34075), followed by analysis of the band strength of the target protein using the CLINX chemiluminescence software. The specific experimental methods and reagent formulations were obtained from the general protocol for Western Blotting (Bio-red, bulletin 6376). The specific experimental methods and reagent formulations were obtained from the general procedure for Western Blotting (Bio-red, bulletin 6376).

### In Situ Hi-C

Based on the previously published In situ Hi-C method with some minor modifications, four Hi-C libraries (as technical replicates) for four pgEpiSCs (biological replicates), and eight Hi-C libraries (as technical replicates) for each of two pEFs (biological replicates) were constructed by the inventors.

In brief, cells (5×10⁶) were cross-linked with formaldehyde at a final concentration of 4% at room temperature for 30 minutes, and then quenched with glycine at a final concentration of 0.25 M/L. Next, the mixture was centrifuged at 1,500×g at room temperature for 10 minutes with the supernatant in combination with lysis buffer and incubation on ice for 15 minutes. The mixture was then centrifuged at 5,000×g at room temperature for 10 minutes. Pellets were washed with NEBuffer 2. The mixture was combined with SDS at a final concentration of 0.1% and incubated at 65° C. for 10 minutes, followed by addition of TritonX-100 at a final concentration of 1% and incubation at 37° C. for 15 minutes. Nuclei were permeabilized, and then DNAs were digested with 200 units of DpnII at 37° C. for 1 hour. The restriction fragment overhangs were filled and labeled with biotinylated nucleotides, followed by linkage in a small volume. After reverse cross-linking, DNAs were purified using Covaris S220 ultrasound instrument with fragments of about 300-500 bp sonicated, and then the linked fragments were pulled down by Dynabeads™-280 streptavidin (Invitrogen, 11206D), followed by terminal repair and addition of poly-A. Next, the adaper was linked and DNA fragments were amplified by PCR using KAPA Hyper Prep Kit (Roche, KK8504) for 8-10 cycles. These fragments were then double-selected using AMPure XP Beads (Beckman, A63882) to separate fragments between 300 and 800 bp, which were ready for sequencing on the DNBSEQ platform (BGI) to provide 100 bp reading segments with paired terminals.

### RNA Sequencing

PgEpiSCs from four donors (as biological replicates) and pEFs from the same dorsal skin region from two donors (as biological replicates), with each replicate having 1×10⁶ cells, were collected and purified by the inventors. Total RNAs were extracted from six samples (four pgEpiSCs and two pEFs) using the RNeasy Mini Kit (Qiagen, 74106). By using the rRNA depletion protocol (globulin-zero gold rRNA removal kit, Illumina, GZG1224) in combination with NEBNext®Ultra™ Directional RNA Library Prep Kit for Illumina® (NEB, E7420S), the libraries for each sample were constructed by the inventors. All libraries were quantified using Qubit dsDNA High Sensitivity Assay Kit (Invitrogen, Life Technologies, Q32851), followed by sequencing on the HiSeq 4000 platform (Illumina).

### ChIP-Seq

Two biological replicates were performed on pgEpiSCs and pEFs by the inventors, with 1×10⁷ cells per sample. ChIP-seq of H3K27ac (a typical histone marker for enhancers) was performed. Cells were cross-linked with formaldehyde at a final concentration of 1% at room temperature for 10 minutes, and then quenched with glycine. Cells were lysed using lysis buffer supplemented with a mixture of protease inhibitors and 1 mM PMSF (finally 1×), with fragments of about 200-500 bp sonicated using Bioruptor. 20 μL of chromatins were stored at −20° C. for DNA input, and 100 μL of chromatins were immunoprecipitated with 5 μg of H3K27ac antibody (Abcam, ab4729) at 4° C. Then, the samples were further incubated for 3 hours with addition of 30 μL protein beads. Beads were washed once with 20 mM Tris/HCl (pH 8.1), 50 mM NaCl, 2 mM EDTA, 1% Triton X-100, and 0.1% SDS. They were treated twice with 10 mM Tris/HCl (pH 8.1), 250 mM LiCl, 1 mM EDTA, 1% NP-40, and 1% deoxycholic acid, and then washed twice with 1× TE buffer (10 mM Tris-Cl at pH 7.5, 1 mM EDTA). The bound material was then eluted from the magnetic beads in 300 μL of elution buffer (100 mM NaHCO₃, 1% SDS), with the first treatment with RNase A at a final concentration of 8 μg/mL at 65° C. for 6 hours, followed by protease K (at a final concentration of 345 μg/mL) at 45° C. overnight. Sequencing libraries were constructed using immunoprecipitated DNA according to the protocol provided by the NEXTflex™ ChIP-Seq kit (Bioo Scientific, NOVA-5143-02). All libraries were sequenced on the HiSeq XTen (Illumina) platform.

### Vector Construction

Three gene editing experiments using different gene editing techniques were conducted by the inventors in pgEpiSCs in order to test whether pgEpiSCs could tolerate continuous gene editing: 1) stable transfection of GFP-NLS cassette using PiggyBac (PB) transposase tool; 2) knock-in (KI) of NANOG-tdTomato reporter gene by CRISPR/Cas9 system; and 3) TYR gene point mutation using cytidine base editor (CBEs).

Firstly, the PB-CMV-EF1A-GFP-NLS plasmid (from Prof. Sen Wu) was constructed by the inventors in order to obtain GFP positive cells, in which the chicken β-actin promoter was replaced with the Homo sapiens elongation factor1 alpha (EF1A) promoter, and GFP-NLS was inserted at the terminal of the EF1A promoter. Secondly, the skeleton of NANOG DNA donor vector having four fragments, with a left homologous arm 3× Flag, 3× Flag-P2A-tdTomato-Loxp-Puro-Loxp, and a right homologous arm, was constructed by the inventors in order to obtain the NANOG-tdTomato KI cell line, as previously mentioned. NEBuilder® HiFi DNA Assembly Master Mix (NEB, E2621X) was used. NANOG sgRNA was targeted in front of the termination codon site to knock in the donor fragment as a reporter gene. The annealed sgRNA sequence was cloned into the BsaI-digested pGL3-U6-sgRNA-PGK-puromycin vector (Addgene, 51133). Finally, the TYR gene was knocked out using the AncBE4max plasmid by the inventors. AncBE4max and pGL3-U6-sgRNA-EGFP vectors were obtained from the Xingxu Huang's laboratory at ShanghaiTech University. SgRNA was synthesized by BGI with an ACCG sequence at the 5′ terminal of the forward primer and an AAAC sequence at the 5′ terminal of the reverse primer. The sgRNA was then annealed and cloned into the pGL3-U6-sgRNA-EGFP vector. Details of the sgRNA sequence were provided in the key resource table.

### Cell Electrotransfection

PgEpiSCs were dissociated using Accutase Cell Dissociation Reagent (Gibco, A11105-01) before electrotransfection. 5×10⁵ cells were transfected at 220 V, 5 ms, 2 pulses using BTX ECM 2001 (Harvard Biosciences, Holliston, MA, USA) for each electroporation. Electroporation was performed using 1 μg PBase helper plasmid and 3 μg PB-CMV-EFIA-GFP-NLS donor plasmid (at a mass ratio of 1:3) for stable transfection of GFP-NLS cassette; using 1 μg pST1374-NLS-flag-linker-Cas9 plasmid (Addgene, 44758), 1 μg NANOG sgRNA plasmid and 1 μg NANOG HMEJ donor plasmid (1 μg for each vector) for NANOG-tdTomato reporter gene knock-in; and using 2 μg ancBE4max vector and 2 μg pGL3-U6-TYR sgRNA-GFP vector (at a mass ratio of 1:1) for TYR gene point mutation. The electrotransfection buffer was provided by Sen Wu's laboratory, at the State Key Laboratory of Agricultural Biotechnology, China Agricultural University. Primers were designed online using NCBI primer BLAST and synthesized by BGI. GFP positive cells were sorted using FACS (MoFlo XDP, Backman) and detected using a 488 nm (710/50 bandpass filter) channel. In order to obtain NANOG-tdTomato positive cells, the transfected cells were selected with puromycin (0.3 μg/mL) and blasticidin (4 μg/mL), followed by picking and amplifying GFP positive clones. DNA was extracted using cell lysis buffer (Invitrogen, AM8723) as a PCR template in order to identify base edited cells. PCR products were sequenced to confirm the point mutation.

Production of Procine Cloned Embryos from pgEpiSCs

Ovaries were collected from slaughterhouses near Beijing. Oocytes with three or four layers of cumulus cells were selected and cultured in IVM solution at 38.5° C., 100% humidity, and 5% CO₂ for 44 hours. IVM stock solution M199 (Sigma, M2154) containing 0.1% L-cysteine (Sigma, C7352-25G), 5% FBS (Gibco, 10099141), 0.1% EGF (Sigma, E9644), 1% penicillin-streptomycin (Gibco, 15140122), and 10% porcine follicular fluid (collected during oocyte collection, centrifuged and filtered, and then stored at −80° C.) was prepared, which was then filtered with a 0.22 μm filter and stored at 4° C. for future use. Before use, 1% GlutaMAX (Gibco, 35050061), 10 IU/mL PMSG, and 10 IU/mL hCG were added. Porcine EpiSCs were differentiated in the basal mudium containing 10 ng/mL BMP4, 5 μM SB431542, and 10 ng/ml FGF2 for more than 1 week, and then used as donor cells for nuclear transfer. Mature oocytes in metaphase II were removed by micromanipulation in HM medium containing 7.5 μg/mL cytochalasin B. Morphologically qualified donor cells were injected into the perisovular space in the fusion mudium (0.3 M/L mannitol, 1.0 mM/L CaCl2, 0.1 mM/L MgCl2, and 0.5 mM/L HEPES) using a BLS electrocell manipulator.

The oocytes were then incubated in PZM-3 for 15 minutes and the fusion rate was assessed under a stereomicroscope. Fifty to sixty fused embryos were placed in a four-well dish with 500 μL PZM-3 in each well and cultured in PZM-3 at 38.5° C. under 5% CO₂, 5% O₂, and 90% N₂, while maintaining maximum humidity. After 24 hours, 150-250 ESCNT zygotes were surgically transplanted into the surrogate mother. The pregnancy status of surrogacy was determined by ultrasound examination at 25-30 days. All cloned piglets were delivered naturally on days 114-120 of gestation.

RESOURCE TABLE

| | | |
|---|---|---|
| REAGENT or RESOURCE | SOURCE | IDENTIFIER |

Antibodies

| | | |
|---|---|---|
| Rabbit polyclonal anti-neuron | Abcam | Cat# ab18207, |
| specific beta III Tubulin | | |
| | | RRID: AB 444319 |
| Rabbit polyclonal anti-alpha | Abcam | Cat# ab5694, |
| smooth muscle Actin | | |
| | | RRID: AB 2223021 |
| Rabbit monoclonal anti-GATA-6 (D61E4) | Cell Signaling Technology | Cat# 5851, |
| | | RRID: AB 10705521 |
| Mouse monoclonal anti-SSEA1 (MC480) | Abcam | Cat# ab16285, |
| | | RRID: AB_870663 |
| Mouse monoclonal anti-SSEA4 (MC813) | Abcam | Cat# ab16287, |
| | | RRID: AB_778073 |
| Mouse monoclonal anti-human-TRA-1-60 | Cell Signaling Technology | Cat# 4746, |
| | | RRID: AB 2119059 |
| Mouse monoclonal anti-human-TRA-1-81 | Cell Signaling Technology | Cat# 4745, |
| | | RRID: AB 2119060 |
| Rabbit polyclonal anti-SOX1 | Abcam | Cat# ab87775, |
| | | RRID: AB 2616563 |
| Rabbit polyclonal anti-Brachyury/Bry | Abcam | Cat# ab20680, |
| | | RRID: AB 727024 |
| Rabbit polyclonal anti-H3K27ac | Abcam | Cat# ab4729; |
| | | RRID: AB 2118291 |
| Rabbit polyclonal anti-TBR2/Eomes | Abcam | Cat# ab23345, |
| | | RRID: AB 778267 |
| Mouse monoclonal anti-Oct-3/4 (C-10) | Santa Cruz Biotechnology | Cat# sc-5279, |
| | | RRID: AB 628051 |
| Mouse monoclonal anti-Sox-2 (E-4) | Santa Cruz Biotechnology | Cat# sc-365823, |
| | | RRID: AB 10842165 |
| Rabbit polyclonal anti-human Nanog | Pepro Tech | Cat# 500-P236, |
| | | RRID: AB 1268274 |
| Rabbit monoclonal anti-Phospho-Stat3 | Cell Signaling Technology | Cat# 52075, |
| (Tyr705) (D3A7) | | RRID: AB 2799407 |
| Rabbit monoclonal anti-Stat3 (D3Z2G) | Cell Signaling Technology | Cat# 12640S, |
| | | RRID: AB_2629499 |
| Rabbit monoclonal anti-GAPDH (D16H11) | Cell Signaling Technology | Cat# 5174, |
| | | RRID: AB 10622025 |
| Goat anti-Mouse IgG (H + L) | Thermo Fisher Scientific | Cat# A-11001, |
| Cross-Adsorbed Secondary | | RRID: AB 2534069 |
| Antibody, Alexa Fluor 488 | | |
| Goat anti-Mouse IgG (H + L) | Thermo Fisher Scientific | Cat# A-11005, |
| Cross-Adsorbed Secondary | | RRID: AB 141372 |
| Antibody, Alexa Fluor 594 | | |
| Donkey anti-Rabbit IgG (H + L) | Thermo Fisher Scientific | Cat# A-21207, |
| Highly Cross-Adsorbed | | RRID: AB 141637 |
| Secondary Antibody, Alexa Fluor 594 | | |
| Donkey anti-Rabbit IgG (H + L) | Thermo Fisher Scientific | Cat# A-21206, |
| Highly Cross-Adsorbed | | RRID: AB 2535792 |
| Secondary Antibody, Alexa Fluor 488 | | |

Chemicals, Peptides, and Recombinant Proteins

| | | |
|---|---|---|
| CHIR99021 | Selleckchem | Cat# S1263 |
| IWR-1-endo | Selleckchem | Cat# S7086 |
| Y-27632 | Selleckchem | Cat# S1049 |
| WH-4-023 | Selleckchem | Cat# S7565 |
| SB431542 | Selleckchem | Cat# S1067 |
| Ruxolitinib (INCB018424) | Selleckchem | Cat# S1378 |
| PD0325901 | Selleckchem | Cat# S1036 |
| Recombinant Murine BMP-4 | Pepro Tech | Cat# 315-27 |
| Human/Murine/Rat Activin A (E. Coli) | Pepro Tech | Cat# 120-14E |
| Recombinant Human LIF | Pepro Tech | Cat# 300-05 |
| Recombinant Human FGF-basic (154 a.a.) | Pepro Tech | Cat# 100-18B |
| All-Trans Retinoic Acid | Pepro Tech | Cat# 3027949 |
| Recombinant Human Noggin | Pepro Tech | Cat# 120-10C |
| Bovine Serum Albumin | Sigma-Aldrich | Cat# A1470 |
| Ascorbic Acid | Sigma-Aldrich | Cat# A4544 |
| KnockOut Serum Replacement | Thermo Fisher Scientific | Cat# A3181502 |
| Neurobasal™ Medium | Thermo Fisher Scientific | Cat# 21103-049 |
| DMEM/F-12, GlutaMAX™ supplement | Thermo Fisher Scientific | Cat# 10565-018 |
| N-2 Supplement (100×) | Thermo Fisher Scientific | Cat# 17502-048 |
| B-27™ Supplement (50×), minus vitamin A | Thermo Fisher Scientific | Cat# 12587-010 |
| GlutaMAX™ Supplement | Thermo Fisher Scientific | Cat# 35050-061 |
| MEM Non-Essential Amino Acids Solution | Thermo Fisher Scientific | Cat# 11140-050 |
| (100×) | | |
| 2-Mercaptoethanol | Thermo Fisher Scientific | Cat# 21985-023 |
| Penicillin-Streptomycin (10,000 U/mL) | Thermo Fisher Scientific | Cat# 15140-122 |
| Gelatin (0.1% in water) | Stem Cell Technologies | Cat# 07903 |
| Trypsin-EDTA (0.05%), phenol red | Gibco | Cat# 25300120 |
| DMEM, high glucose, no glutamine | Gibco | Cat# 11960-044 |
| Fetal bovine serum (FBS) | Gibco | Cat# 16000-044 |
| Accutase cell dissociation reagent | Gibco | Cat# A11105-01 |
| TrypL™ Express | Gibco | Cat# 12605010 |
| Dulbecco's phosphate- | Gibco | Cat# C14190500CP |
| buffered saline (DPBS) | | |

Critical Commercial Assays

| | | |
|---|---|---|
| KAPA Hyper Prep Kits | KAPA Biosystems | Cat# KK8054 |
| RNeasy Mini Kit | QIAGEN | Cat# 74106 |
| Globin-Zero Gold rRNA Removal Kit | Illumina | Cat# GZG1224 |
| NEBNext® Ultra™ Directional | New England Biolabs | Cat# E7760 |
| RNA Library Prep Kit | | |
| for Illumina® | | |
| NEXTflex™ ChIP-Seq Kit | Bioo Scientific | Cat# NOVA-5143-02 |
| RNAprep pure Cell/Bacteria Kit | TIANGEN | Cat# DP430 |
| 5 × All-In-One RT Master Mix | ABM | Cat# G490 |
| TIANamp Genomic DNA Kit | TIANGEN | Cat# DP304 |
| Rapid Giemsa staining kit | BBI Life Sciences | Cat# E202FA0001 |
| Gel & PCR Clean Up Kit | OMEGA | Cat# D2000-02 |
| Lipofectamine™ 3000 Transfection Kit | Invitrogen | Cat# 2173193 |
| Lipofectamine™ Stem Transfection Reagent | Invitrogen | Cat# STEM00008 |
| Endo-free Plasmid Mini Kit | OMEGA | Cat# D6950-02B |
| Bradford Protein Assay Kit | BIO-RED | Cat# 5000201 |
| 2 × RealStar Green Power Mixture | GenStar | Cat# A311-05 |
| Cells-to-cDNA™ II Kit | Invitrogen | Cat# AM8723 |
| Deposited Data | | |
| Human ESCs Hi-C data | (Dixon et al., 2015; | GEO: GSE52457, |
| | Lyn et al., 2018) | GSE: 105028 |
| Human dermal fibroblasts Hi-C data | (Nir et al., 2018) | GEO: GSE123552 |
| Mouse ESCs Hi-C data | (Bonev et al., 2017: | GEO: GSE96107, |
| | Mclaughlin et al., 2019) | GSE: 124342 |
| Mouse embryonic fibroblasts Hi-C data | (Colognori et al., 2019; Di | GEO: GSE113339 |
| | Giammartino et al., 2019) | |
| Human ESCs RNA-seq data | (Ji et al, 2016) | GEO: GSE69692 |
| Human dermal fibroblasts RNA-seq data | (Consortium, 2012) | GEO: GSE78670 |
| Mouse ESCs RNA-seq data | (Shakla et al., 2020) | GEO: GSE121171 |
| Mouse embryonic fibroblasts RNA-seq data | (Di Giammartino et al., | GEO: GSE113431 |
| | 2019) | |
| Human formative PSCs, | (Kinoshita et al., 2021) | GEO:GSE131556 |
| mouse naive, formative and | | |
| primed PSCs RNA-seq data | | |
| Human naive and conventional | (Guo et al., 2017) | Array Express: |
| PSCs RNA-seq data | | E-MTAB-5674 |
| Human naive and conventional | (Ji et al., 2016) | GEO:GSE69692 |
| PSCs RNA-seq data | | |
| Experimental Models: Organisms | | |
| CD-1® (ICR) IGS Mice | Vital River | 201 |
| BALB/c Nude Mice | Vital River | 401 |
| BAMA pigs | Beijing Farm Animal | N/A |
| | Research Center | |
| Nongda pigs | CAU Experimental | N/A |
| | Miniature Pig Farm | |
| DLY pigs | YAU/NEAU Experimental | N/A |
| | Pig Farm | |

Oligonucleotides

| | |
|---|---|
| TSO primer: | N/A |
| AAGCAGTGGTATCAACGCAGAGTACATrGrG+G | |
| (SEQ ID NO: 1) | |
| ISPCR: | N/A |
| AAGCAGTGGTATCAACGCAGAGT(SEQ ID NO: 2) | |
| 3′-P2: | N/A |
| GTGACTGGAGTTCAGACGTGTGCTCTTCCGATC( | |
| (SEQ ID NO: 3) | |
| short universal primer: | N/A |
| AATGATACGGCGACCACCGAGATCTACACTCTT | |
| TCCCTACACGAC(SEQ ID NO: 4) | |
| illunima-QP2: | N/A |
| CAAGCAGAAGACGGCATACGA(SEQ ID NO: 5) | |
| POU5F1: | N/A |
| Forword: CAAACTGAGGTGCCTGCCCTTC(SEQ ID | |
| NO: 6) | |
| Reverse: ATTGAACTTCACCTTCCCTCCAACC(SEQ | |
| ID NO: 7) | |
| NANOG: | N/A |
| Forword: CATCTGCTGAGACCCTCGAC (SEQ ID | |
| NO: 8) | |
| Reverse: GGGCTTGTGGAAGAATCAGG (SEQ ID | |
| NO: 9) | |
| SOX2: | N/A |
| Forword: CATCAACGGTACACTGCCTCTC (SEQ ID | |
| NO: 10) | |
| Reverse: ACTCTCCTCCCATTTCCCTCTTT (SEQ ID | |
| NO: 11) | |
| REX1(ZFP42): | N/A |
| Forword: GGGATCACGTGTGTGCAGAA (SEQ ID | |
| NO: 12) | |
| Reverse: TTCCTGCGACAGCCTTCAAA (SEQ ID | |
| NO: 13) | |
| CDH1: | N/A |
| Forword: GACTTCTGCCAGAGGAACCC (SEQ ID | |
| NO: 14) | |
| Reverse: CACTGGCCCCATGTGTTAGT (SEQ ID | |
| NO: 15) | |
| IGF2: | N/A |
| Forword: CGTGCTGCTATGCTGCTTAC (SEQ ID | |
| NO: 16) | |
| Reverse: AAGCAGCACTCTTCCACGAT (SEQ ID | |
| NO: 17) | |
| SNAI2: | N/A |
| Forword: CCCACACCCTACCTTGTGTC (SEQ ID | |
| NO: 18) | |
| Reverse: TGACATCCGAGTGTGTCTGC (SEQ ID | |
| NO: 19) | |
| SRC: | N/A |
| Forword: GCCAACATCCTGGTTGGAGA (SEQ ID | |
| NO: 20) | |
| Reverse: ATCCCAAAGGACCACACGTC (SEQ ID | |
| NO: 21) | |
| WNT5A: | N/A |
| Forword: CGCGAAGACAGGCATCAAAG (SEQ ID | |
| NO: 22) | |
| Reverse: CCTATCTGCATGACCCTGCC (SEQ ID | |
| NO: 23) | |
| GATA6: | N/A |
| Forword: CACTACTTGTGCAACCGCTG (SEQ ID | |
| NO: 24) | |
| Reverse: TTCTGCGGCTTTATGAGGGG (SEQ ID | |
| NO: 25) | |
| HAND1: | N/A |
| Forword: TGCGAGTGCATACCTTCTGT (SEQ ID | |
| NO: 26) | |
| Reverse: AGGCCCTGAGGGGAGTTTAT (SEQ ID | |
| NO: 27) | |
| PAX6: | N/A |
| Forword: TGTCCAACGGATGTGTGAGT (SEQ ID | |
| NO: 28) | |
| Reverse: TCTGTCTCGGATTTCCCAA (SEQ ID | |
| NO: 29) | |
| CDH2: | N/A |
| Forword: GCCTCAAGCCAACCTTACCT (SEQ ID | |
| NO: 30) | |
| Reverse: AGCTCTTGAGGAAAAGGCCC (SEQ ID | |
| NO: 31) | |
| ID2: | N/A |
| Forword: TCGCACCCCACTATTGTCAG (SEQ ID | |
| NO: 32) | |
| Reverse: TTCAGAAGCCTGCAAGGACA (SEQ ID | |
| NO: 33) | |
| ID3: | N/A |
| Forword: CATCTTCCCATCCAGACAGCC (SEQ ID | |
| NO: 34) | |
| Reverse: GTCAAGTGGGCACGACAAAG (SEQ ID | |
| NO: 35) | |
| BMP2: | N/A |
| Forword: GTCTTTCGGGAGCAGACACA (SEQ ID | |
| NO: 36) | |
| Reverse: GTCACCAACCTGGTGTCCAA (SEQ ID | |
| NO: 37) | |
| EOMES: | N/A |
| Forword: ACTCCCATGGACCTCCAGAA (SEQ ID | |
| NO: 38) | |
| Reverse: TCGCTTACAAGCACTGGTGT (SEQ ID | |
| NO: 39) | |
| T: | N/A |
| Forword: GAAGTACGTGAACGGGGAGT(SEQ ID | |
| NO: 40) | |
| Reverse: CACGATGTGGATTCGAGGCT(SEQ ID | |
| NO: 41) | |
| LIN28A: | N/A |
| Forword: TGCCGGCATCTGTAAATGGT (SEQ ID | |
| NO: 42) | |
| Reverse: ACTCTGGTGCACAAAGACGT (SEQ ID | |
| NO: 43) | |
| C-MYC: | N/A |
| Forword: ATCCAAGACCACCACCACTG (SEQ ID | |
| NO: 44) | |
| Reverse: GTTCACAGCAACATTCAGGTAGA (SEQ | |
| ID NO: 45) | |
| ETV4: | N/A |
| Forword: AGAACCGGCCAGCTATGAAC (SEQ ID | |
| NO: 46) | |
| Reverse: ATTGTCCGGGAAAGCCAGAG (SEQ ID | |
| NO: 47) | |
| ETV5: | N/A |
| Forword: TCAGCACATGGGTTCCAGTC (SEQ ID | |
| NO: 48) | |
| Reverse: CCTTCATGGCTGCTGGAGAA (SEQ ID | |
| NO: 49) | |
| BMP 4: | N/A |
| Forword: TTCATTTTAGGAGCCATTCTGTAGT (SEQ | |
| ID NO: 50) | |
| Reverse: TCCTAGCAGGACTTGGCATAAT(SEQ ID | |
| NO: 51) | |
| EF-1α: | N/A |
| Forword: AATGCGGTGGGATCGACAAA (SEQ ID | |
| NO: 52) | |
| Reverse: CACGCTCACGTTCAGCCTTT (SEQ ID | |
| NO: 53) | |
| TYR gene sgRNA targeting sequence: | N/A |
| Forword: ACCGACCTCAGTTCCCCTTCACCG (SEQ | |
| ID NO: 54) | |
| Reverse: AAACCGGTGAAGGGGAACTGAGGT | |
| (SEQ ID NO: 55) | |
| TYR gene mutant site test primer: | N/A |
| Forword: CTGGACTTTCCAGACTTCCG (SEQ ID | |
| NO: 56) | |
| Reverse: GTGCAGTTGGGTCCCTGAAA (SEQ ID | |
| NO: 57) | |
| GFP test primer | N/A |
| Forword: CAACACCCGCATCGAGAAGT (SEQ ID | |
| NO: 58) | |
| Reverse: ACCACGAAGCTGTAGTAGCC (SEQ ID | |
| NO: 59) | |
| NANOG knock-in 3′ ARM test primer | N/A |
| Forword: GCAACCTCCCCTTCTACGAG (SEQ ID | |
| NO: 60) | |
| Reverse: GTGAAGCACACGGTAGGGTA (SEQ ID | |
| NO: 61) | |
| NANOG knock-in 5′ ARM test primer | N/A |
| Forword: TGTCCATTGCTGAAGCATGTAAT (SEQ | |
| ID NO: 62) | |
| Reverse: GAAGTTAGTAGCTCCGCTTCCTG (SEQ | |
| ID NO: 63) | |

### Example 1

### 1. ScRNA Seq Revealing Lineage Separation During Porcine Embryonic Development

In order to reveal the molecular basis of lineage separation and pluripotency changes during porcine embryonic development, scRNA-seq was performed by the inventors according to a modified single-cell tagged reverse transcription sequencing (STRT-seq) protocol (see STAR Methods) (Gao et al., 2018; Wang et al., 2018), and a total of 1,458 single cell data were screened from porcine oocytes and embryos sampled from E1 to E14 after quality control (FIG. 1-1A and FIG. 1-2A). The cell types at different stages were identified by the inventors using Shared nearest neighbor (SNN) algorithm and t-distributed stochastic neighbor embedding (t-SNE) (Stuart et al., 2019), and different lineage differentiation processes were described. Based on known differentiation and pluripotency marker genes, these cells were classified into specific embryonic lineages at different embryonic stages (FIG. 1-1B and FIG. 1-2B) (Edgar et al., 2013; Nakamura et al., 2016; Ramos-Ibeas et al., 2019). Cell populations with different gene expression characteristics were formed based on functional enrichment of specific expressed genes and co-expressed gene networks (FIG. 1-2C and FIG. 1-3A).

The inventors found that the first lineage separation of porcine embryos started at E5, the late stage of morula (FIG. 1-1C). Two subgroups of cells at the late stage of morula, known as pre-ICM and pre-TE, exhibited differential expression of classical precursor marker genes for ICMs (such as PDGFRA) or TEs (such as DAB2) (Petropoulos et al., 2016; Wei et al., 2018; Wu et al., 2016) (FIG. 1-1C). At E6, the early stage of blastula, ICM and TE cells were divided into two cell populations, with PDGFRA, NANOG and SOX2 upregulated in ICMs and CDX2, DAB2, GATA2 and GATA3 upregulated in TEs (FIG. 1-1C). Heterogeneous expressions of GATA6 (a hyperblast marker) and NANOG (an epiblast marker) were detected in ICMs of E6 (FIG. 1-1D), indicating the beginning of the second lineage separation (Plusa et al., 2008; Saiz et al., 2016).

At E7, the late stage of blastula, GATA6 and NANOG positive cells were divided into two populations (FIG. 1-1D). The results showed that the second lineage separation was completed, and lineages of hyperblast cells (GATA4 and GATA6) and epiblast cells (NANOG; and SOX2) were established in the embryo. From E7 to E10, the number of epiblast and hyperblast cells increased rapidly, and hyperblast cells extended within TE to form a complete cavity called the gastrocoele (Oestrup et al., 2009); and the epiblast cell pluripotentcy marker genes NANOG, POU5F1 and SOX2 were highly expressed (FIG. 1-1D) until the formation of the mesderm began at E11 (marked by the up-regulation of gastralization marker genes such as LEF1, KDR, TBXT, HANDI and BMP4) (FIG. 1-1E). Based on previously reported histological and morphological evidence, the molecular genetic regulatory trends observed by the inventors during the development of E0-E14 were completely consistent with the well-defined patterns during development (Kobayashi et al., 2017; Oestrup et al., 2009). This consistency highlighted the representativeness and practicality of single-cell transcriptome sequencing data provided by the inventors, which might be used to support basic research in applied biotechnology exploration and embryo regulation.

### 2. Tracking Changes of Pluripotency During Porcine Epiblast Development

In order to track the pluripotency changes during porcine epiblast development and determine their effect on signaling pathways, 11,113 DEGs from E4 early morulas, E5 pre-ICMs, E6 ICMs, E7-E10 epiblasts, and E11-E14 ectoderms were pairwise compared by the inventors, and divided into 36 clusters based on gene expression trends at the embryonic stage (see STAR Methods). Interestingly, the expression of representative naïve pluripotent genes (such as ESRRB, KLF4, LIFR, STAT3, TFCP2L1 and TBX3) was significantly reduced from E4 early morulas to E7 epiblasts. In contrast, classical primed pluripotent genes (such as ZIC5, ETV5, ZIC2, LEF1, BMP4 and LIN28B) were upregulated after E7 (FIG. 1-1F). Formative marker genes (such as TDGF1, DNMT3A, OTX2, KLF5, LIN28A and NODAL) had higher expression levels in the epiblast at E7-E10, while their expression levels were lower in the exoderm at E11-E13 (FIG. 1-1F). In addition, annotations on the expression trends of the three pluripotent states were supported by pluripotent regulatory networks and rich functional terminology (FIG. 1-3A and FIG. 1-3B). A rapid loss of naïve pluripotency before the formation of the epiblast at E7 was observed by the inventors, which might help explain why the establishment of porcine naïve embryonic stem cells failed in previous studies. In addition, these data indicated that epiblast maintained a stable formative state from E7 to E10 and might support the establishment of stable pluripotent stem cell lines.

The inventors hypothesized that the isolation and establishment of stem cell lines derived from the epiblast required stable pluripotent signaling pathways and inhibition of differentiation signals. Finally, the inventors focused on investigating four signaling pathways: JAK/STAT3, Activin/Nodal, FGF/ERK and Wnt/β-catenin. The inventor found that the hub genes of the JAK/STAT3 signaling pathway were highly expressed in E4 early morulas to E6 ICMs cells, but sharply decreased after E7 epiblast formation (FIG. 1-1G), consistent with the expression pattern of naïve pluripotency marker genes during porcine epiblast development (FIG. 1-1F). Activin A and FGF2 receptors were highly expressed in the epiblast from E7 to E10 (FIG. 1-1G), indicating that the proliferation and maintenance of porcine epiblast cells required the presence of Activin A and FGF2. Interestingly, the inventors noticed that the activity of Wnt/β-catenin signaling significantly increased during the transition from epiblast to ectoderm development from E10 to E11 (FIG. 1-1G), indicating that the inhibition of Wnt/β-catenin signaling pathway might be necessary for inducing and maintaining stable porcine epiblast.

### Example 2

1. Establishment of a Stable Porcine pgEpiSC Line Using Epiblast Cells at E10

### (1) Study Procedures

Based on Example 1, the conclusion drawn by the inventors from scRNA-seq analysis suggested that the embryonic gastralization should be prevented by the small molecule inhibitors related to WNT signaling for the establishement of pgEpiSCs, and epiblast self-renewal was promoted by cytokines in TGF-β superfamily and FGF family. In order to achieve this objective in epiblast cells at E10, the inventors tested various medium formulations that combined multiple inhibitors and cytokines, and ultimately developed a conditioned medium consisting of three inhibitors (CHIR99021, IWR1-endo and WH-4-023) and three cytokines (LIF, Activin A and FGF2), which was referred to as “3i/LAF” medium by the inventors (FIG. 2A). The inventors found that the establishment efficiency of epiblast cells at E10 stage was higher than that of epiblast cells at other stage under 3i/LAF conditions (FIG. 2B and FIG. 2C). PgEpiSCs from E10 epiblast might be passaged by enzymatic hydrolysis at the single-cell level, with passage rates ranging from 1:3 to 1:5/2-3 days. The proliferation doubling time of pgEpiSCs was about 16 hours (FIG. 2D and FIG. 2E), with the efficiency of single-cell colony formation of about 33.83% (FIG. 2F)

The inventors found that pgEpiSCs retained their dome like clone morphology (FIG. 2G), AP positive (FIG. 2H), and normal karyotype (FIG. 3-2a). PgEpiSCs expressed pluripotent stem cell markers such as POU5F1, NANOG and SOX2 during long-term culture in vitro (FIG. 2I), and pluripotent surface markers including SSEA1, SSEA4, TRA-1-81 and TRA-1-60 (FIG. 2J). Importantly, the embryonic body (EB) differentiation test showed that pgEpiSCs might differentiate into three germ layers after removal of inhibitors and cytokines from the medium (FIG. 2K). Directional induced differentiation test showed that pgEpiSCs might also differentiate into the expected tripoblastic layers in the conditioned medium (FIG. 2L). Teratoma formation test confirmed that pgEpiSCs developed into the expected tripoblastic layers in vivo (FIG. 2M). All pgEpiSC lines might be passaged more than 60 times without altering the above characteristics. By comparing the karyotypes, SNVs, and Short InDels (≤30 bp) of pgEpiSC cell lines between different passages of the same cell line and different donor cell lines, the inventors found that the cell lines had normal karyotypes and very few gene mutations were detected after 60 passages during the culture (FIG. 3-2b to FIG. 3-2g). The inventors then randomly selected two cell lines for long-term maintenance ability tests, which had been passaged at least 216 times.

These results indicated that the stable porcine pgEpiSC lines had been successfully established by the inventors.

### (2) Specific Steps

1) PgEpiSCs were derived from the porcine embryonic epiblast at E8-E10, where the hypoblast and TE were mechanically removed as much as possible to ensure that no hypoblast or TE was adhered to the surface of epiblast. There were two methods to establish pgEpiSCs as follows: (1) the epiblast was treated with TrypLE™ Express for 3 minutes, and then dispersed into small cell clusters by pipetting repeatedly using a pipet with a diameter of 40-50 μm. Subsequently, the cell clusters were seeded in a 12-well dish (with the feeder cells at a density of about 2×10⁵ cells/well) with 750 μL 3i/LAF medium (note: 3i/LAF medium supplemented with 10 μM Y27632) and cultured in an incubator at 37° C., humidity of 100%, 5% CO₂, and 5% O₂ (at a low or normal concentration of oxygen). \textcircled{2} The whole epiblast was seeded into a dish and cultured under the same conditions. Stable cell lines could be obtained by digestion and passaging at P2 by both methods, while the first method was more efficient. After 12 hours, 750 μL 3i/LAF medium was further added without changing the medium, and after 24 hours, 3i/LAF medium (containing 2 μM Y27632) was refreshed. Subsequently, the medium was refreshed every 12 hours, and the morphology of the clones was observed after 2-3 days of clonal growth.
2) During passage, the cell surface was washed with DPBS warmed at 37° C. after firstly removal of the medium, followed by addition of an appropriate amount (depending on the well size of the plate, generally, at least 500 μL required by covering the surface of the 6-well plate, and so on for other well sizes of plates) of Accutase cell dissociation reagent (Gibco, A11105-01). After digestion in an incubator at 37° C. for 3-5 minutes, once most of the clones fell off, the digestion might be terminated by adding an equal amount of medium, followed by gently pipetting 10-15 times to fully disperse the cell clones, and then seeding depending on the passage ratio of 1:3-1:5. And 10 μM Y27632 was added into the 3i/LAF medium used during passage.
3) When the stable cell lines were cultured normally, the medium was refreshed 12 hours after passage (as appropriate, if Accutase had not been removed by centrifugation after digestion, the medium was refreshed after 12 hours; and if Accutase had been removed by centrifugation after digestion, the medium was refreshed after 24 hours), and again after 24 hours, and then the medium was refreshed every 12 hours until passage.
4) During cryopreservation, the cells were digested into single cells according to the above method, and centrifuged at 1,000 rpm for 5 minutes to collect cells with removal of the supernatant, and then gently resuspended using freshly prepared pEpiSCs cell cryopreservation solution (10% DMSO+90% KOSR) pre-cooled at 4° C., which were divided into cryotubes depending on the passage rate, labeled with cell informations, and transferred into a cell cryopreservation box pre-cooled at 4° C., and then quickly transferred into a −80° C. ultra-low temperature freezer and after 24 hours, transferred into liquid nitrogen for long-term storage.
5) When thawing, the cells were taken out from liquid nitrogen and placed with a fixing frame in water bath at 37° C. under quickly shaking to thaw until only a small amount of ice crystals remaining in the cryopreservation tube. The cell cryopreservation solution was carefully transferred into a 15 mL centrifuge tube containing 3 mL cell culture solution in a clean bench after spraying with 75% alcohol for disinfection, followed by centrifugion at 1,000 rpm for 5 minutes with removal of the supernatant, and then the cells were gently resuspended and seeded onto the feeder layer cells according to the desired density.
   2. Specific Preparation and Application of “3i/LAF” Medium

When preparing 500 mL of 3i/LAF medium, the components contained in the basal medium (BM) and their respective amounts or final concentrations in 500 mL 3i/LAF medium were as follows: 227.5 mL DMEM/F12 (Thermo Fisher 729 Scientific, 10565-018), 227.5 mL Neurobasal (Thermo Fisher Scientific, 21103-049), 2.5 mL N2 supplement (Thermo Fisher Scientific, 17502-048), 5 mL B27 supplement (Thermo Fisher Scientific, 12587-010), 0.5% GlutaMAX (Thermo Fisher Scientific, 35050-061), 1% non-essential amino acids (Thermo Fisher 732 Scientific, 11140-050), 0.1 mM β-mercaptoethanol (Thermo Fisher Scientific, 21985-023), 1% penicillin-streptomycin (Thermo FisherScientific, 15140-122), 5% knockout serum replacement (KOSR, Thermo Fisher Scientific, A3181502, optional), and 50 μg/mL ascorbic acid (Sigma-Aldrich, A4544). In order to prepare 3i/LAF medium, it was necessary to further add small molecules and cytokines to the above basal medium at the final concentrations as follows in 500 mL 3i/LAF medium: CHIR99021 (1 μM, Selleckchem, S1263), IWR-1-endo (2.5 μM, Selleckchem, S7086), WH-4-023 (1 μM, Selleckchem, S7565), recombinant human LIF (10 ng/mL, PeproTech, 300-05), recombinant human Activin A (25 ng/mL, Peprotech, 120-14E), and recombinant human FGF-basic (10 ng/mL, Peprotech, 100-18B).

To promote the proliferation of pgEpiSCs, it was possible to further add ROCK inhibitor Y-27632 (at a final concentration of 10 μM for passage; at a final concentration of 2 μM for maintenance; Selleckchem, S1049) to the previously prepared 3i/LAF medium.

Porcine pgEpiSCs were cultured in 3i/LAF medium under the conditions of 20% O₂, 5% CO₂ and 38.5° C.

PgEpiSCs were cultured on mouse embryonic fibroblast (MEF) feeder cells (5×10⁴ cells/cm²) treated with mitomycin C (Selleckchem, S8146). The medium was refreshed every 12 hours with addition of fresh 3i/LAF medium.

3. Requirement of Inhibitors and Growth Factors for Long-Term Maintenance of pgEpiSCs

Based on the prepared 3i/LAF medium and its culture method in2 above, the requirement of various factors for long-term maintenance in vitro of pgEpiSCs was test by the inventors under the same conditions as in 2 above, except for removing small molecule inhibitors and cytokines from the medium one by one.

The inventors found that removal of any of the three WNT signaling pathway related inhibitors would disrupt the desired dome clone morphology and weaken the alkaline phosphatase (AP) staining signal intensity (FIG. 3-1A), and also upregulate genes related to epithelial mesenchymal transition (EMT), including IGF2, SNAI2, SRC and WNT5A (FIG. 3-1B). In particular, removal of IWR-1-endo directly led to unclear boundaries of pgEpiSC clones and significant downregulation of core pluripotency factors such as NANOG, POU5F1, SOX2 and REX1 (FIG. 3-1B).

In addition, removal of IWR-1-endo or WH-4-023 might lead to differentiation of the mesoderm and endoderm in pgEpiSCs, manifested by upregulation of the gastrulation formation marker genes BMP2, BMP4, EOMES and T (FIG. 3-1B), and result in reduced or heterogeneous accumulation of the pluripotency factor POU5F1, while promoting the expression of the mesoderm and endoderm progenitor cell marker EOMES (FIG. 3-1C). CHIR99021 played a dual role in maintaining pluripotency in both mouse and human PSCs, with promoting self-renewal at the low concentration and promoting differentiation at the high concentration. Removal of CHIR99021 downregulated the expression of cell proliferation related genes such as LIN28A, C-MYC, ETV4 and ETV5 (FIG. 3-1D), indicating impaired proliferation of pgEpiSC. High concentrations of CHIR99021 resulted in downregulation of the pluripotency marker POU5F1 and significant upregulation of the endodermal marker GATA6 (FIG. 3-1E), which was further confirmed by immunofluorescence staining (FIG. 3-1F), indicating that CHIR99021 was conserved in PSCs of mice, humans, and pigs.

The detection after removal of cytokines from 3i/LAF indicated that removal of Activin A, a member of the TGF-β superfamily, resulted in reduced levels of the pluripotency marker NANOG (FIG. 3-1G and FIG. 3-1H). In order to further support the role of Activin A in long-term culture of pgEpiSCs, SB431542, a universal inhibitor of TGF-β, was added to the medium (to avoid the impact of feeder secretions), resulting in irregular clone morphology and a significant decline in NANOG levels (FIG. 3-1G and FIG. 3-1H). Notably, the addition of SB431542 also resulted in a significant decrease in pluripotency markers (such as POU5F1 and REX1), and a significant increase in BMP4 and BMP downstream transcription factors (such as ID2 and ID3) that mediated the induction of primitive streaks during embryonic development (Kurek et al., 2015; Valdez Magana et al., 2014) (FIG. 3-1H).

When FGF2 was removed (with the addition of ERK/MEK inhibitor PD0325901), pgEpiSCs could not proliferate or passage normally (FIG. 3-1I to FIG. 3-1K). The inventors also noted that under otherwise unchanged conditions, the reduced concentration of FGF2 significantly reduced the proliferation ability of pgEpiSCs (FIG. 3-1K), and the low concentration of FGF2 was not conducive to cell proliferation, while cells proliferated faster at the high concentration. Notably, the inventors found that LIF was not a necessary condition for maintaining the morphology of pgEpiSC clones (FIG. 3-1L), but the addition of the JAK1/2 inhibitor ruxolitinib made the clones flattened. Western blotting showed that phosphorylated STAT3 could only be detected in the presence of LIF (FIG. 3-1M), indicating that pgEpiSCs could respond to the pluripotency-promoting effect of LIF.

In addition, based on the prepared 3i/LAF medium and its culture method in 2 above, the following tests were also performed by the inventors under the same conditions as in 2 above.

(1) Under the Same Conditions, Except for Only Replacing IWR-1-Endo with XAV939

After replacing IWR-1-endo with XAV939, the cells were unable to maintain pluripotency for a long time, and AP positivity was lost around 8 passages (as shown in FIG. 3-3A).

After replacing IWR-1-endo with XAV939, the expression of core pluripotency factor OCT4 (POU5F1) was downregulated; and the expression of pluripotency factors REX1, STELLA, and ESRRB was downregulated (as shown in FIG. 3-3B).

### (2) Under the Same Conditions, Except for Only Adjusting the Concentration of CHIR99021

The concentration of CHIR99021 in the culture system affected the pluripotency and homogeneity of cells. When the concentration of CHIR99021 was high, the expression of the pluripotency gene POU5F1 was reduced, and the expression of the lineage differentiation gene GATA6 was upregulated, presenting a heterogeneous expression pattern. The expression levels of the mesodermal marker gene TBX3 and the endodermal marker gene ESRRB increased with increasing concentration (as shown in FIG. 3-3C and FIG. 3-3D).

### (3) Under the Same Conditions, Except for Only Adjusting the Concentration Ratio of CHIR99021 to IWR-1-Endo

The concentration ratio of CHIR99021 to IWR-1-endo had an impact on pluripotency, with the optimal ratio of C/I≈1:2-1:3, and the expression of the pluripotency factor Nanog was the highest (as shown in FIG. 3-3E).

4. Extended Study of the “3i/LAF” Medium

According to the 3i/LAF medium and its culture method prepared in 2 above, the inventors also investigated the effect of the adjusted or replaced medium on the long-term in vitro maintenance of pgEpiSCs on the basis of adjusting the concentrations of the small molecule inhibitors and cytokines or replacing the compositions of the small molecule inhibitors and cytokines in the culture medium one by one without altering the other conditions.

(1) Adjusting the Concentration of Small Molecule Inhibitors and Cytokines in 3i/LAF Medium

Medium adjustment: compared with the 3i/LAF medium and its culture method prepared in 2 above, only the concentrations of small molecule inhibitors or cytokines in the 3i/LAF medium were single-factor adjusted as shown in Table A below, respectively, and the other conditions were kept unchanged.

Experimental method: pgEpiSCs were cultured with the adjusted medium, after which AP staining was performed on the cultured pgEpiSCs of P1 (indicating the pgEpiSCs after 1 passage in the adjusted medium) or P3 (indicating the pgEpiSCs after 3 passages in the adjusted medium). The specific steps of AP staining are described in the section of Experimental Methods described previously.

Principle of the experiment: undifferentiated stem cells will express Ap at high level. By staining fixed stem cells, differentiated cells show no color and undifferentiated cells show purple or red color.

Experimental result: the results are shown in Table A or FIGS. 3-4A to 3-4F below.

TABLE A
Adjustment of the concentrations of small molecule inhibitors
or cytokines in 3i/LAF medium and test results

| Group No. | | | | | |
|---|---|---|---|---|---|
| of the | | | | | |
| adjusted | Passage of the | Small molecule | | | Test results on |
| medium | pgEpiSCs | inhibitors or cytokines | Concentration | | pluripotency |
| 1 | P1 | CHIR99021 | 0.01 | μM | As shown in FIG. |
| | | | 1 | μM | 3-4A, left panel |
| | | | 3 | μM | |
| | P3 | CHIR99021 | 0.01 | μM | As shown in FIG. |
| | | | 1 | μM | 3-4A, right panel |
| | | | 3 | μM | |
| 2 | P1 | IWR-1-endo | 1 | μM | As shown in FIG. |
| | | | 2.5 | μM | 3-4B, left panel |
| | | | 3 | μM | |
| | P3 | IWR-1-endo | 1 | μM | As shown in FIG. |
| | | | 2.5 | μM | 3-4B, right panel |
| | | | 3 | μM | |
| 3 | P1 | WH-4-023 | 0.01 | μM | As shown in FIG. |
| | | | 1 | μM | 3-4C, left panel |
| | | | 5 | μM | |
| | P3 | WH-4-023 | 0.01 | μM | As shown in FIG. |
| | | | 1 | μM | 3-4C, right panel |
| | | | 5 | μM | |
| 4 | P1 | Recombinant human | 0.01 | ng/mL | As shown in FIG. |
| | | Activin A | 25 | ng/mL | 3-4D, left panel |
| | | | 100 | ng/mL | |
| | P3 | Recombinant human | 0.01 | ng/mL | As shown in FIG. |
| | | Activin A | 25 | ng/mL | 3-4D, right panel |
| | | | 100 | ng/mL | |
| 5 | P1 | Recombinant human FGF- | 1 | ng/mL | As shown in FIG. |
| | | basic | 25 | ng/mL | 3-4E, left panel |
| | | | 100 | ng/mL | |
| | P3 | Recombinant human FGF- | 1 | ng/mL | As shown in FIG. |
| | | basic | 25 | ng/mL | 3-4E, right panel |
| | | | 100 | ng/mL | |
| 6 | P1 | Recombinant human LIF | 1 | ng/mL | As shown in FIG. |
| | | | 25 | ng/mL | 3-4F, left panel |
| | | | 100 | ng/mL | |
| | P3 | Recombinant human LIF | 1 | ng/mL | As shown in FIG. |
| | | | 25 | ng/mL | 3-4F, right panel |
| | | | 100 | ng/mL | |

It can be seen from FIGS. 3-4A˜3-4F that the medium obtained by adjusting the concentration of small molecular inhibitors or cytokines in 3i/LAF medium as above can still maintain the pluripotency of pgEpiSCs in vitro for a long time.

(2) Replacing the Small Molecular Inhibitors and Cytokines Components in 3i/LAF Medium

Medium adjustment: compared with the 3i/LAF medium and its culture method prepared in 2 above, only the small molecular inhibitors or cytokines components in the 3i/LAF medium were replaced by single factor as shown in Table B below, and other conditions remain unchanged.

Experimental method: pgEpiSCs were cultured with the adjusted medium, after which AP staining was performed on the cultured pgEpiSCs of P1 (indicating the pgEpiSCs after 1 passage in the adjusted medium) or P3 (indicating the pgEpiSCs after 3 passages in the adjusted medium). The specific steps of AP staining are described in the section of Experimental Methods described previously.

Principle of the experiment: undifferentiated stem cells will express Ap at high level. By staining fixed stem cells, differentiated cells show no color and undifferentiated cells show purple or red color.

Experimental result: the results are shown in Table B or FIGS. 3-4G to 3-4I below.

TABLE B
Replacement of small molecular inhibitors or cytokines
components in 3i/LAF medium and test results

| Group | | | | | |
|---|---|---|---|---|---|
| No. | | | | | |
| of the | | | | | |
| adjusted | Passage of the | Small molecule inhibitors or | | | Test results on |
| medium | pgEpiSCs | cytokines | Concentration | | pluripotency |
| 7 | P1 | CHIR99021 was replaced with | 0.1 | ng/mL | As shown in |
| | | WNT3a | (0.0025 | nM) | FIG. 3-4G, left |
| | | | 1 | ng/mL | panel |
| | | | (0.025 | nM) | |
| | | | 10 | ng/mL | |
| | | | (0.25 | nM) | |
| | P3 | CHIR99021 was replaced with | 0.1 | ng/mL | As shown in |
| | | WNT3a | (0.0025 | nM) | FIG. 3-4G, left |
| | | | 1 | ng/mL | panel |
| | | | (0.025 | nM) | |
| | | | 10 | ng/mL | |
| | | | (0.25 | nM) | |
| 8 | P1 | WH-4-023 was replaced with | 3 | nM | As shown in |
| | | A419259 | 300 | nM | FIG. 3-4H, left |
| | | | 30 | μM | panel |
| | P3 | WH-4-023 was replaced with | 3 | nM | As shown in |
| | | A419259 | 300 | nM | FIG. 3-4H, |
| | | | 30 | μM | right panel |
| 9 | P1 | Recombinant human Activin A | 0.1 | ng/mL | As shown in |
| | | was replaced with Nodal | 1 | ng/mL | FIG. 3-41, left |
| | | | 10 | ng/mL | panel |
| | P3 | Recombinant human Activin A | 0.1 | ng/mL | As shown in |
| | | was replaced with Nodal | 1 | ng/mL | FIG. 3-41, right |
| | | | 10 | ng/mL | panel |

It can be seen from FIGS. 3-4G˜3-4I that the medium obtained by replacing the small molecular inhibitors or cytokines components in 3i/LAF medium as above can still maintain the pluripotency of pgEpiSCs in vitro for a long time.

### Example 3: The Correlation Between the Transcriptome of pgEpiSC and Pre-Gastrulation Epiblast Cells

To investigate the transcriptome characteristics of pgEpiSCs, we performed scRNA-seq on the passages 10 and 60 (i.e. low- and high-passage) of pgEpiSCs, and then compared the transcriptome of pgEpiSCs with that of porcine embryonic single cells (from E0 to E14). The tSNE visualization showed that pgEpiSCs were clustered together, independent of cells from E0 to E14 at each stage of the embryo (FIG. 4A). The expression levels of marker genes in lineage separation indicated that epiblast specific genes (NANOG, TDGF1, ETV4, GDF3 and NODAL, etc.) were highly consistently expressed in pgEpiSCs (FIG. 4B), indicating that pgEpiSCs maintained the transcriptome characteristics of epiblast cells. Principal component analysis (PCA) using differentiation and pluripotent DEGs showed that pgEpiSCs were clustered in embryonic cells at E10 (FIG. 4C). Pairwise correlation analysis showed that pgEpiSCs at low- and high-passage exhibited very consistently (r=0.97, P<2.2×10⁻¹⁶, Spearman's rank correlation), showing collectively the most similarity to the epiblast at E10 compared to other embryonic stages of epiblast or ectoderm cells (average r=0.88, P<2.2×10 to 16, Spearman's rank correlation) (FIG. 4D). In addition, the expression levels of typical pluripotency and gastrulation marker genes in pgEpiSCs indicated that they were closest to the epiblast layer at E10 (FIG. 4E). All these results indicated that pgEpiSCs had transcriptome characteristics similar to their origin in the epiblast layer at E10.

Next, we compared the RNA-seq data from pgEpiSCs with those from human naïve, conventional, and formative PSCs, as well as mouse naïve, primed, and previously reported formative PSCs by transcriptome analysis (Guo et al., 2017; Ji et al., 2016; Kinoshita et al., 2021). We found that pgEpiSCs were more similar to formative and primed (or conventional) PSCs than naïve PSCs (FIG. 4F). Importantly, pgEpiSCs exhibited stronger expression of formative hPSC specific genes compared to conventional and naïve hPSCs (FIG. 4G). Compared to conventional hESCs, representative genes with increased expression were shown in Table 4-1, while representative genes with decreased expression were shown in Table 4-2.

TABLE 4-1
Representative genes with increased expression
compared to conventional hESC

| | Expression | Expression | |
|---|---|---|---|
| | levels in | levels in | pgEpiSC/conventional |
| Genes | conventional hESC | pgEpiSC | hESC |
| ADPRM | 2.66 | 6.21 | 2.33 |
| FRG1 | 2.51 | 5.23 | 2.08 |
| GAS2 | 2.05 | 5.49 | 2.68 |
| HK3 | 0.15 | 5.13 | 35.23 |
| NCAN | 2.65 | 6.16 | 2.32 |
| POU5F1B | 1.29 | 10.62 | 8.25 |
| ZFP2 | 1.43 | 6.76 | 4.74 |
| CLDND2 | 0.83 | 5.56 | 6.68 |
| CRK | 2.43 | 9.58 | 3.94 |
| DMP1 | 0.23 | 6.26 | 27.42 |
| GATD3B | 0.95 | 5.31 | 5.62 |
| H3F3A | 3.55 | 8.48 | 2.39 |
| IRF8 | 0.69 | 8.06 | 11.72 |
| ITGA4 | 2.47 | 5.26 | 2.13 |
| KRT14 | 0.08 | 7.65 | 90.77 |
| MPC1 | 3.04 | 6.38 | 2.10 |
| MSH4 | 0.64 | 6.83 | 10.60 |
| NDE1 | 1.90 | 6.36 | 3.35 |
| PBX2 | 3.24 | 6.54 | 2.02 |
| PRKY | 2.17 | 6.02 | 2.77 |
| RGL2 | 3.26 | 7.78 | 2.39 |
| SOX10 | 2.35 | 5.07 | 2.15 |
| VHLL | 0.03 | 5.19 | 178.33 |

TABLE 4-2
Representative genes with reduced expression
compared to conventional hESC

| | Expression | Expression | |
|---|---|---|---|
| | levels in | levels in | pgEpiSC/conventional |
| Genes | conventional hESC | pgEpiSC | hESC |
| ABCC4 | 5.18 | 0.38 | 0.07 |
| ADCY2 | 6.03 | 2.02 | 0.33 |
| AK2 | 6.29 | 0.21 | 0.03 |
| AKT1 | 6.25 | 0.01 | 0.00 |
| BMP2 | 5.10 | 1.40 | 0.27 |
| CD46 | 7.49 | 0.08 | 0.01 |
| CDH3 | 7.54 | 2.84 | 0.38 |
| DNM1 | 5.09 | 1.98 | 0.39 |
| DPPA4 | 9.26 | 0.81 | 0.09 |
| ETS1 | 5.99 | 1.99 | 0.33 |
| GAB2 | 5.11 | 2.06 | 0.40 |
| ID2 | 5.51 | 1.56 | 0.28 |
| KDR | 6.81 | 0.26 | 0.04 |
| MMP24 | 5.72 | 1.38 | 0.24 |
| TGFB1 | 5.64 | 2.52 | 0.45 |
| VGLL3 | 5.02 | 1.55 | 0.31 |
| ZNF195 | 6.86 | 0.13 | 0.02 |
| ZNF519 | 5.36 | 0.13 | 0.02 |

The results above supported the successful establishment of the pgEpiSCs cell line, and also indicated that pgEpiSCs had the characteristics and pluripotency of pre-embryonic epithelial cells at E10.

### Example 4: Spatial Regulatory Characteristics of pgEpiSC Transcription

Using high-deep in situ high-throughput chromatin conformation capture (hic) sequencing technology, we reconstructed the three-dimensional genomic structure of pgEpiSCs and porcine embryonic fibroblasts (pEFs), combined with data from 16 replicates, and ultimately obtained a map with a maximum resolution of 300 bp. We found that the chromatin spatial plasticity of pgEpiSCs was higher than that of pEFs (reflected in the lower degree of chromosome intermingle in pgEpiSCs compared to pEFs: 0.18/0.71, P<2.2×10⁻¹⁶, Wilcoxon rank test) (FIGS. 5A-5B). Based on the high entropy state, the chromatin of pgEpiSCs was more disordered (1.57/1.00, P=5.63×10⁻⁴, Wilcoxon rank test) (FIGS. 5C-5D), which was consistent with previous studies on human and murine (Lindsly et al., 2021; Tan et al., 2018). Thus, the typical loose regulatory architecture we detected in the high-deep in situ Hi-C analysis might be due to the establishment of pluripotency by pgEpiSCs.

Furthermore, we investigated how the relatively loose heterochromatin regulatory structure of pgEpiSCs might affect the transcription process of pluripotency. We found that there were fewer promoter-enhancer interactions (PEIs) in pgEpiSCs compared to PEF (pgEpiSCs, 20,389 enhancers assigned to 6,498 promoters) (PEF, 30,852 enhancers assigned to 7,823 promoters). There was a significant transcriptome difference between pgEpiSCs and pEFs, sharing only 5,547 PEIs. We calculated the regulatory potential score (RPS) for each promoter, which was an index based on spatial proximity that represented the joint regulatory effect of multiple enhancers on a given gene (Cao et al., 2017; Fulco et al., 2019; Whalen et al., 2016). For a given promoter, its RPS calculation formula was Σn (log 10In), wherein In was the normalized interaction intensity of PEI n of the promoter. A total of 875 co-variant genes with RPS and gene expression were identified (i.e., genes with higher RPS values in pgEpiSCs were usually upregulated compared to pEFs (log 2 fold change [FC]>1, FDR<0.05)), where 75 genes were strongly expressed in pgEpiSCs (TPM>5 compared to TPM<0.5 in pEFs). Table 5 provided examples of representative co-variant genes strongly expressed in pgEpiSCs.

TABLE 5
Representative co-variant genes strongly expressed in pgEpiSCs

| | | | | Log₂ |
|---|---|---|---|---|
| | | | | (fold change) of |
| | Gene | TPM in | TPM in | expression |
| Ensembl ID | symbol | pEpiSCs | pEFs | (pEpiSCs/pEFs) |
| ENSSSCG00000003557 | LIN28A | 435.21 | 0.00 | 15.85 |
| ENSSSCG00000007252 | DNMT3B | 263.29 | 0.22 | 9.46 |
| ENSSSCG00000032299 | LEFTY2 | 193.44 | 0.02 | 12.85 |
| ENSSSCG00000035444 | ZFP42 | 174.32 | 0.09 | 10.69 |
| ENSSSCG00000038188 | | 124.69 | 0.06 | 10.66 |
| ENSSSCG00000033478 | | 106.46 | 0.00 | 11.88 |
| ENSSSCG00000000252 | KRT8 | 85.84 | 0.15 | 8.67 |
| ENSSSCG00000040403 | NANOG | 65.01 | 0.13 | 8.46 |
| ENSSSCG00000000253 | KRT18 | 58.26 | 0.03 | 10.32 |
| ENSSSCG00000008429 | EPCAM | 47.52 | 0.01 | 11.46 |
| ENSSSCG00000017934 | CLDN7 | 38.33 | 0.22 | 6.59 |
| ENSSSCG00000026894 | NFE2L3 | 26.68 | 0.20 | 6.33 |
| ENSSSCG00000025652 | CDH1 | 25.30 | 0.02 | 9.40 |
| ENSSSCG00000005063 | OTX2 | 24.00 | 0.00 | 13.32 |
| ENSSSCG00000004679 | SORD | 23.84 | 0.31 | 5.53 |
| ENSSSCG00000031204 | | 22.27 | 0.00 | 11.61 |
| ENSSSCG00000022739 | DSG2 | 19.28 | 0.35 | 5.10 |
| ENSSSCG00000028804 | CCDC181 | 18.11 | 0.12 | 6.67 |
| ENSSSCG00000021207 | HESX1 | 17.57 | 0.30 | 5.37 |
| ENSSSCG00000040222 | | 17.50 | 0.31 | 5.83 |
| ENSSSCG00000036181 | | 15.62 | 0.12 | 6.73 |
| ENSSSCG00000039049 | MAP7 | 15.44 | 0.02 | 9.32 |
| ENSSSCG00000021307 | USP44 | 14.65 | 0.03 | 8.29 |
| ENSSSCG00000007718 | CLDN4 | 13.89 | 0.03 | 7.70 |
| ENSSSCG00000012003 | | 13.64 | 0.33 | 4.51 |
| ENSSSCG00000010771 | UTF1 | 12.92 | 0.00 | 11.98 |
| ENSSSCG00000002361 | VRTN | 12.12 | 0.00 | 13.88 |
| ENSSSCG00000015246 | ST14 | 11.63 | 0.00 | 13.66 |
| ENSSSCG00000013469 | ZNF555 | 11.01 | 0.27 | 4.78 |
| ENSSSCG00000021767 | | 10.52 | 0.02 | 8.79 |
| ENSSSCG00000002884 | LSR | 10.38 | 0.01 | 9.72 |
| ENSSSCG00000006195 | PRDM14 | 9.95 | 0.00 | 12.47 |
| ENSSSCG00000008902 | PPAT | 9.92 | 0.24 | 4.66 |
| ENSSSCG00000017915 | VMO1 | 9.89 | 0.07 | 6.43 |
| ENSSSCG00000002456 | CHGA | 9.78 | 0.01 | 10.21 |
| ENSSSCG00000031347 | | 9.78 | 0.20 | 4.82 |
| ENSSSCG00000016841 | SLC1A3 | 9.72 | 0.11 | 5.65 |
| ENSSSCG00000000766 | CECR2 | 9.61 | 0.12 | 5.47 |
| ENSSSCG00000009429 | TNFSF11 | 9.32 | 0.38 | 3.54 |
| ENSSSCG00000002032 | SLC7A8 | 9.30 | 0.08 | 5.75 |
| ENSSSCG00000009120 | ZGRF1 | 9.16 | 0.42 | 3.94 |
| ENSSSCG00000008618 | MYCN | 8.95 | 0.02 | 8.01 |
| ENSSSCG00000008465 | KCNG3 | 8.88 | 0.01 | 9.49 |
| ENSSSCG00000023861 | PFAS | 8.77 | 0.20 | 4.63 |
| ENSSSCG00000032831 | BRI3BP | 8.68 | 0.12 | 5.48 |
| ENSSSCG00000006099 | ESRP1 | 8.57 | 0.02 | 7.84 |
| ENSSSCG00000021259 | CDA | 8.42 | 0.06 | 6.38 |
| ENSSSCG00000026718 | PLCH1 | 7.93 | 0.03 | 7.75 |
| ENSSSCG00000021941 | ZSCAN21 | 7.82 | 0.43 | 3.60 |
| ENSSSCG00000007479 | SALL4 | 7.81 | 0.01 | 9.34 |
| ENSSSCG00000000298 | PPP1R1A | 7.78 | 0.08 | 5.94 |
| ENSSSCG00000007602 | BAIAP2L1 | 7.75 | 0.11 | 5.11 |
| ENSSSCG00000026996 | ABCC4 | 7.75 | 0.46 | 3.10 |
| ENSSSCG00000037485 | SOX2 | 7.63 | 0.00 | 11.05 |
| ENSSSCG00000009859 | TESC | 7.61 | 0.46 | 3.28 |
| ENSSSCG00000039169 | CER1 | 7.42 | 0.00 | 12.34 |
| ENSSSCG00000031675 | | 7.41 | 0.06 | 6.35 |
| ENSSSCG00000006050 | | 7.22 | 0.12 | 4.89 |
| ENSSSCG00000009584 | SEMA4D | 7.11 | 0.26 | 3.81 |
| ENSSSCG00000037744 | ZNF483 | 7.04 | 0.31 | 3.77 |
| ENSSSCG00000037612 | LIN28B | 6.96 | 0.08 | 5.62 |
| ENSSSCG00000011610 | NUP21 | 6.85 | 0.00 | 10.70 |
| ENSSSCG00000038138 | TFEC | 6.84 | 0.11 | 5.51 |
| ENSSSCG00000016140 | FZD5 | 6.62 | 0.07 | 5.53 |
| ENSSSCG00000011257 | ACVR2B | 6.55 | 0.21 | 4.24 |
| ENSSSCG00000034904 | | 6.36 | 0.00 | 5.97 |
| ENSSSCG00000025838 | AP1M2 | 6.13 | 0.00 | 12.70 |
| ENSSSCG00000034478 | | 6.09 | 0.00 | 7.22 |
| ENSSSCG00000014450 | TCOF1 | 5.76 | 0.39 | 2.99 |
| ENSSSCG00000011277 | CCK | 5.67 | 0.02 | 10.23 |
| ENSSSCG00000004621 | MYO5C | 5.64 | 0.01 | 8.86 |
| ENSSSCG00000025672 | RAVER2 | 5.57 | 0.32 | 3.44 |
| ENSSSCG00000033197 | | 5.56 | 0.00 | 5.17 |
| ENSSSCG00000034739 | | 5.44 | 0.01 | 8.85 |
| ENSSSCG00000004318 | RRAGD | 5.35 | 0.06 | 5.83 |

In addition, we detected that OTX2 (as well as LIN28A, NANOG, PRDM14, SALL4, UTF1 and ZFP42) interacted specifically with enhancers in pgEpiSCs, while enhancers were absent in pEFs (FIGS. 5E-5K).

### Example 5: Continuous Gene Editing of pgEpiSCs and Production of Cloned Piglets

One of the main limitations of using porcine somatic cell nuclear transfer currently was that donor cells might usually only support single round genome editing (Yan et al., 2018). To test whether pgEpiSCs might tolerate continuous genome editing, we performed experiments on various forms of genome manipulation (FIG. 6A)

Firstly, GFP-nls reporter fragments were stably transfected to obtain pgEpiSCs, with the rate of GFP-positive cells detected by flow cytometry of 21.27% (FIG. 6B). Secondly, using these GFP positive cells, we performed CRISPR/cas9 mediated knock-in, specifically inserting the tdTomato reporter gene cassette into the NANOG locus, located directly in front of the natural termination codon, known as GFP-NANOG-tdTomato pgEpiSCs (GN-pgEpiSCs) (FIG. 6C). According to NANOG-tdTomato fluorescence screening, GN-pgEpiSC clones were screened and then re-amplified in 3i/LAF medium (FIG. 6D). Consistent with the known state of NANOG (a pluripotency marker), no tdTomato reporter fluorescence was detected after experimental induction of differentiation on the edited pgEpiSCs after knock-in (FIG. 6E). For the third and final genomic modification, we performed c-t transition using cytosine base editors (CBEs) (Koblan et al., 2018; Komor et al., 2016) at the termination codon of the TYR site, inducing a albinism known to be related to pig hair color (Li et al., 2018; Xie et al., 2019). Sequencing analysis was performed on 99 clones, where 24.24% (24/99) of them were heterozygous and 3.03% (3/99) were homozygous (the C-T base editing in the GNT-pgEpiSCs background is referred to as GNT-pgEpiSCs) (FIG. 6F). These results indicated that pgEpiSCs could tolerate continuous genomic modifications, including conventional transgenic insertions, precise insertion by CRISPR/Cas9, and single base transition editing by CBEs.

Then, we conducted a nuclear transfer experiment and exclusively used wild-type (WT) pgEpiSCs, GFP-pgEpiSCs, and GNT-pgEpiSCs as nuclear donor cells to obtain cloned embryos. We further mixed and transplanted 200 WT pgEpiSCs and 203 GFP-pgEpiSCs cloned embryos, as well as 660 GNT-pgEpiSCs cloned embryos (FIG. 6G). We ultimately obtained 1 WT pgEpiSCs cloned piglet, 1 GFP-pgEpiSCs cloned piglet, and 3 GNT-pgEpiSCs cloned piglets (FIG. 6H). The cloning efficiency of gene edited pgEpiSC was similar to that of wild-type pgEpiSC cells and comparable to that of fibroblasts (FIG. 6G). Importantly, GNT-pgEpiSCs cloned piglets exhibited the expected albino coat color phenotype (FIG. 6I). These results indicated that pgEpiSCs could tolerate continuous multi-gene editing and had the potential to generate complex pig models.

The specific examples described above provide further detailed explanations of the purpose, technical solution, and beneficial effects of the present invention. It should be understood that the examples above are only specific examples of the present invention and are not intended to limit the scope of protection of the present invention. Any modifications, equivalent replacements, improvements and the like made within the spirit and principles of the present invention should be included in the scope of protection of the present invention.

1. A medium comprising:
a first component that is IWR-1-endo;
a second component that is selected from WH-4-023, A419259; and
a third component that is selected from fibroblast growth factors.

2. The medium of claim 1, wherein the medium further comprises:
a fourth component that is selected from CHIR99021, WNT3a;
a fifth component that is selected from TGF-β superfamily members; and
a sixth component that is LIF.

3. The medium of claim 1, wherein the medium has one or more of technical features in (1) to (5) below:
(1) the second component is WH-4-023;
(2) the third component is selected from FGF2, FGF1;
preferably, the third component is FGF2;
more preferably, the third component is recombinant human FGF2.
(3) the fourth component is CHIR99021;
(4) the fifth component is selected from Activin A, Nodal;
preferably, the fifth component is Activin A;
more preferably, the fifth component is recombinant human Activin A;
(5) the sixth component is selected from recombinant human LIF, recombinant mouse LIF;
more preferably, the sixth component is recombinant human LIF.

4. The medium of claim 1, wherein the medium has one or more of technical features in (1) to (7) below:
(1) the first component has a concentration of 0.1-10 μM;
preferably, the first component has a concentration of 0.9-3 μM;
more preferably, the first component has a concentration of 2.5 μM;
(2) the second component has a concentration of 3 nM-30 PM;
preferably, the second component has a concentration of 0.01-5 μM;
more preferably, the second component has a concentration of 1 μM;
(3) the third component has a concentration of 0.01-100 ng/mL;
preferably, the third component has a concentration of 1-100 ng/ml;
more preferably, the third component has a concentration of 10 ng/ml;
(4) the fourth component has a concentration of 0.01-3 μM;
preferably, the fourth component has a concentration of 0.01-3 μM;
more preferably, the fourth component has a concentration of 1 μM;
(5) the fifth component has a concentration of 0.01-100 ng/ml;
preferably, the fifth component has a concentration of 25 ng/ml;
(6) the sixth component has a concentration of 0.01-100 ng/ml;
preferably, the sixth component has a concentration of 1-100 ng/ml;
more preferably, the sixth component has a concentration of 10 ng/ml;
(7) the concentration ratio of the fourth component to the first component is 25:1-1:25;
preferably, the concentration ratio of the fourth component to the first component is 2:3-1:3.

5. The medium of claim 1, wherein the medium further comprises a seventh component that is a ROCK inhibitor;
preferably, the seventh component is Y-27632;
preferably, the seventh component has a concentration of 0.01-50 μM;
preferably, when the medium is used for cell passage, the seventh component has a concentration of 0.01-20 μM, preferably 10 μM;
preferably, when the medium is used for cell maintenance, the seventh component has a concentration of 0.01-10 μM, preferably 2 μM.

6. The medium of claim 1, wherein the medium further comprises an eighth component that is a basal medium;
preferably, the basal medium is used to culture mammalian (preferably porcine) pluripotent stem cells;
more preferably, the basal medium comprises a minimal medium, N2 supplement, B27 supplement, non-essential amino acids, β-mercaptoethanol, knockout serum replacement, and any one selected from GlutaMAX, glutamine;
further preferably, the basal medium comprises a minimal medium, N2 supplement, B27 supplement, non-essential amino acids, β-mercaptoethanol, knockout serum replacement and GlutaMAX;
most preferably, the basal medium comprises a minimal medium, N2 supplement, B27 supplement, non-essential amino acids, β-mercaptoethanol, knockout serum replacement, ascorbic acid, GlutaMAX and penicillin-streptomycin;
preferably, the minimal medium is selected from DMEM/F12, Neurobasal, DMEM, KO-DMEM, RPMI1640, MEM, mTeSR1, or any combination thereof;
preferably, the minimal medium is selected from DMEM/F12, Neurobasal, or a combination thereof;
preferably, the minimal medium is DMEM/F12 and Neurobasal.

7. The medium of claim 6, wherein the basal medium has one or more of technical features in (1) to (12) below:
(1) the minimal medium has a volume fraction of 1%-99%, preferably 91%;
(2) the DMEM/F12 has a volume fraction of 1%-99%, preferably 45%-50% (such as 45.5%);
(3) the Neurobasal has a volume fraction of 1%-99%, preferably 45%-50% (such as 45.5%);
(4) the N2 supplement has a volume fraction of 0.002%-10%, preferably 0.5%;
(5) the B27 supplement has a volume fraction of 0.002%-20%, preferably 1%;
(6) the non-essential amino acids have a volume fraction of 0.01%-10%, preferably 1%;
(7) the β-mercaptoethanol has a concentration of 0.01 mM-1 mM, preferably 0.1 mM;
(8) the knockout serum replacement has a volume fraction of 0.01%-50%, preferably 5%;
(9) the ascorbic acid has a concentration of 1 μg/mL-5000 μg/mL, preferably 50 μg/mL;
(10) the GlutaMAX or glutamine (preferably GlutaMAX) has a volume fraction of 0.01%-10%, preferably 0.5%;
(11) the penicillin-streptomycin has a volume fraction of 0.01%-20%, preferably 1%;
(12) the volume ratio of the DMEM/F12 to the Neurobasal is 5:1-1:5, preferably 1:1.

8. A method for preparing mammalian pluripotent stem cells, comprising:
1) providing a mammalian embryonic epiblast or its inner cell mass;
2) culturing the mammalian embryonic epiblast or its inner cell mass using the medium of claim 1 to obtain mammalian pluripotent stem cells;
preferably, the mammal is a pig;
preferably, the mammalian embryonic epiblast is E8 to E10 (such as E8, E9, or E10) mammalian embryonic epiblast.

9. A method for culturing mammalian pluripotent stem cells and/or maintaining their pluripotency, comprising:
1. providing mammalian pluripotent stem cells;
2. culturing the mammalian pluripotent stem cells using the medium of claim 1;
preferably, the mammal is a pig;
preferably, the mammalian pluripotent stem cells are porcine embryonic pre-gastrulation epiblast stem cells.

10. The method of claim 8, wherein the method is performed in the presence of feeder cells;
preferably, the feeder cells are selected from mouse embryonic fibroblasts or STO cells;
preferably, the feeder cells are mouse embryonic fibroblasts;
preferably, the feeder cells are mouse embryonic fibroblasts with cell division arrest;
preferably, the feeder cells are mouse embryonic fibroblasts treated with mitomycin C.

11. The method of claim 9, wherein the method is performed in the presence of feeder cells;
preferably, the feeder cells are selected from mouse embryonic fibroblasts or STO cells;
preferably, the feeder cells are mouse embryonic fibroblasts;
preferably, the feeder cells are mouse embryonic fibroblasts with cell division arrest;
preferably, the feeder cells are mouse embryonic fibroblasts treated with mitomycin C.


SEQUENCE LISTING (63 sequences)

SEQ ID NO: 1 - DNA, 30 bp, synthetic construct
  source 1..30: mol_type = other DNA; note = TSO primer
  misc_feature 28: note = n = riboguanosine (rG)
  misc_feature 29: note = n = riboguanosine (rG)
  misc_feature 30: note = n = Locked nucleic acid (LNA)-modified G (+G)
aagcagtggtatcaacgcagagtacatnnn

SEQ ID NO: 2 - DNA, 23 bp, synthetic construct
  source 1..23: mol_type = other DNA; note = ISPCR
aagcagtggtatcaacgcagagt

SEQ ID NO: 3 - DNA, 33 bp, synthetic construct
  source 1..33: mol_type = other DNA; note = 3'-P2
gtgactggagttcagacgtgtgctcttccgatc

SEQ ID NO: 4 - DNA, 45 bp, synthetic construct
  source 1..45: mol_type = other DNA; note = short universal primer
aatgatacggcgaccaccgagatctacactctttccctacacgac

SEQ ID NO: 5 - DNA, 21 bp, synthetic construct
  source 1..21: mol_type = other DNA; note = illunima-QP2
caagcagaagacggcatacga

SEQ ID NO: 6 - DNA, 22 bp, synthetic construct
  source 1..22: mol_type = other DNA; note = POU5F1:Forword
caaactgaggtgcctgcccttc

SEQ ID NO: 7 - DNA, 25 bp, synthetic construct
  source 1..25: mol_type = other DNA; note = POU5F1:Reverse
attgaacttcaccttccctccaacc

SEQ ID NO: 8 - DNA, 20 bp, synthetic construct
  source 1..20: mol_type = other DNA; note = NANOG:Forword
catctgctgagaccctcgac

SEQ ID NO: 9 - DNA, 20 bp, synthetic construct
  source 1..20: mol_type = other DNA; note = NANOG:Reverse
gggcttgtggaagaatcagg

SEQ ID NO: 10 - DNA, 22 bp, synthetic construct
  source 1..22: mol_type = other DNA; note = SOX2:Forword
catcaacggtacactgcctctc

SEQ ID NO: 11 - DNA, 23 bp, synthetic construct
  source 1..23: mol_type = other DNA; note = SOX2:Reverse
actctcctcccatttccctcttt

SEQ ID NO: 12 - DNA, 20 bp, synthetic construct
  source 1..20: mol_type = other DNA; note = REX1(ZFP42):Forword
gggatcacgtgtgtgcagaa

SEQ ID NO: 13 - DNA, 20 bp, synthetic construct
  source 1..20: mol_type = other DNA; note = REX1(ZFP42):Reverse
ttcctgcgacagccttcaaa

SEQ ID NO: 14 - DNA, 20 bp, synthetic construct
  source 1..20: mol_type = other DNA; note = CDH1:Forword
gacttctgccagaggaaccc

SEQ ID NO: 15 - DNA, 20 bp, synthetic construct
  source 1..20: mol_type = other DNA; note = CDH1:Reverse
cactggccccatgtgttagt

SEQ ID NO: 16 - DNA, 20 bp, synthetic construct
  source 1..20: mol_type = other DNA; note = IGF2:Forword
cgtgctgctatgctgcttac

SEQ ID NO: 17 - DNA, 20 bp, synthetic construct
  source 1..20: mol_type = other DNA; note = IGF2:Reverse
aagcagcactcttccacgat

SEQ ID NO: 18 - DNA, 20 bp, synthetic construct
  source 1..20: mol_type = other DNA; note = SNAI2:Forword
cccacaccctaccttgtgtc

SEQ ID NO: 19 - DNA, 20 bp, synthetic construct
  source 1..20: mol_type = other DNA; note = SNAI2:Reverse
tgacatccgagtgtgtctgc

SEQ ID NO: 20 - DNA, 20 bp, synthetic construct
  source 1..20: mol_type = other DNA; note = SRC:Forword
gccaacatcctggttggaga

SEQ ID NO: 21 - DNA, 20 bp, synthetic construct
  source 1..20: mol_type = other DNA; note = SRC:Reverse
atcccaaaggaccacacgtc

SEQ ID NO: 22 - DNA, 20 bp, synthetic construct
  source 1..20: mol_type = other DNA; note = WNT5A:Forword
cgcgaagacaggcatcaaag

SEQ ID NO: 23 - DNA, 20 bp, synthetic construct
  source 1..20: mol_type = other DNA; note = WNT5A:Reverse
cctatctgcatgaccctgcc

SEQ ID NO: 24 - DNA, 20 bp, synthetic construct
  source 1..20: mol_type = other DNA; note = GATA6:Forword
cactacttgtgcaaccgctg

SEQ ID NO: 25 - DNA, 20 bp, synthetic construct
  source 1..20: mol_type = other DNA; note = GATA6:Reverse
ttctgcggctttatgagggg

SEQ ID NO: 26 - DNA, 20 bp, synthetic construct
  source 1..20: mol_type = other DNA; note = HAND1:Forword
tgcgagtgcataccttctgt

SEQ ID NO: 27 - DNA, 20 bp, synthetic construct
  source 1..20: mol_type = other DNA; note = HAND1:Reverse
aggccctgaggggagtttat

SEQ ID NO: 28 - DNA, 20 bp, synthetic construct
  source 1..20: mol_type = other DNA; note = PAX6:Forword
tgtccaacggatgtgtgagt

SEQ ID NO: 29 - DNA, 19 bp, synthetic construct
  source 1..19: mol_type = other DNA; note = PAX6:Reverse
tctgtctcggatttcccaa

SEQ ID NO: 30 - DNA, 20 bp, synthetic construct
  source 1..20: mol_type = other DNA; note = CDH2:Forword
gcctcaagccaaccttacct

SEQ ID NO: 31 - DNA, 20 bp, synthetic construct
  source 1..20: mol_type = other DNA; note = CDH2:Reverse
agctcttgaggaaaaggccc

SEQ ID NO: 32 - DNA, 20 bp, synthetic construct
  source 1..20: mol_type = other DNA; note = ID2:Forword
tcgcaccccactattgtcag

SEQ ID NO: 33 - DNA, 20 bp, synthetic construct
  source 1..20: mol_type = other DNA; note = ID2:Reverse
ttcagaagcctgcaaggaca

SEQ ID NO: 34 - DNA, 21 bp, synthetic construct
  source 1..21: mol_type = other DNA; note = ID3:Forword
catcttcccatccagacagcc

SEQ ID NO: 35 - DNA, 20 bp, synthetic construct
  source 1..20: mol_type = other DNA; note = ID3:Reverse
gtcaagtgggcacgacaaag

SEQ ID NO: 36 - DNA, 20 bp, synthetic construct
  source 1..20: mol_type = other DNA; note = BMP2:Forword
gtctttcgggagcagacaca

SEQ ID NO: 37 - DNA, 20 bp, synthetic construct
  source 1..20: mol_type = other DNA; note = BMP2:Reverse
gtcaccaacctggtgtccaa

SEQ ID NO: 38 - DNA, 20 bp, synthetic construct
  source 1..20: mol_type = other DNA; note = EOMES:Forword
actcccatggacctccagaa

SEQ ID NO: 39 - DNA, 20 bp, synthetic construct
  source 1..20: mol_type = other DNA; note = EOMES:Reverse
tcgcttacaagcactggtgt

SEQ ID NO: 40 - DNA, 20 bp, synthetic construct
  source 1..20: mol_type = other DNA; note = T:Forword
gaagtacgtgaacggggagt

SEQ ID NO: 41 - DNA, 20 bp, synthetic construct
  source 1..20: mol_type = other DNA; note = T:Reverse
cacgatgtggattcgaggct

SEQ ID NO: 42 - DNA, 20 bp, synthetic construct
  source 1..20: mol_type = other DNA; note = LIN28A:Forword
tgccggcatctgtaaatggt

SEQ ID NO: 43 - DNA, 20 bp, synthetic construct
  source 1..20: mol_type = other DNA; note = LIN28A:Reverse
actctggtgcacaaagacgt

SEQ ID NO: 44 - DNA, 20 bp, synthetic construct
  source 1..20: mol_type = other DNA; note = C-MYC:Forword
atccaagaccaccaccactg

SEQ ID NO: 45 - DNA, 23 bp, synthetic construct
  source 1..23: mol_type = other DNA; note = C-MYC:Reverse
gttcacagcaacattcaggtaga

SEQ ID NO: 46 - DNA, 20 bp, synthetic construct
  source 1..20: mol_type = other DNA; note = ETV4:Forword
agaaccggccagctatgaac

SEQ ID NO: 47 - DNA, 20 bp, synthetic construct
  source 1..20: mol_type = other DNA; note = ETV4:Reverse
attgtccgggaaagccagag

SEQ ID NO: 48 - DNA, 20 bp, synthetic construct
  source 1..20: mol_type = other DNA; note = ETV5:Forword
tcagcacatgggttccagtc

SEQ ID NO: 49 - DNA, 20 bp, synthetic construct
  source 1..20: mol_type = other DNA; note = ETV5:Reverse
ccttcatggctgctggagaa

SEQ ID NO: 50 - DNA, 25 bp, synthetic construct
  source 1..25: mol_type = other DNA; note = BMP4:Forword
ttcattttaggagccattctgtagt

SEQ ID NO: 51 - DNA, 22 bp, synthetic construct
  source 1..22: mol_type = other DNA; note = BMP4:Reverse
tcctagcaggacttggcataat

SEQ ID NO: 52 - DNA, 20 bp, synthetic construct
  source 1..20: mol_type = other DNA; note = EF-1(alpha):Forword
aatgcggtgggatcgacaaa

SEQ ID NO: 53 - DNA, 20 bp, synthetic construct
  source 1..20: mol_type = other DNA; note = EF-1(alpha):Reverse
cacgctcacgttcagccttt

SEQ ID NO: 54 - DNA, 24 bp, synthetic construct
  source 1..24: mol_type = other DNA; note = TYR gene sgRNA targeting sequence: Forword
accgacctcagttccccttcaccg

SEQ ID NO: 55 - DNA, 24 bp, synthetic construct
  source 1..24: mol_type = other DNA; note = TYR gene sgRNA targeting sequence: Reverse
aaaccggtgaaggggaactgaggt

SEQ ID NO: 56 - DNA, 20 bp, synthetic construct
  source 1..20: mol_type = other DNA; note = TYR gene mutant site test primer: Forword
ctggactttccagacttccg

SEQ ID NO: 57 - DNA, 20 bp, synthetic construct
  source 1..20: mol_type = other DNA; note = TYR gene mutant site test primer: Reverse
gtgcagttgggtccctgaaa

SEQ ID NO: 58 - DNA, 20 bp, synthetic construct
  source 1..20: mol_type = other DNA; note = GFP test primer Forword
caacacccgcatcgagaagt

SEQ ID NO: 59 - DNA, 20 bp, synthetic construct
  source 1..20: mol_type = other DNA; note = GFP test primer Reverse
accacgaagctgtagtagcc

SEQ ID NO: 60 - DNA, 20 bp, synthetic construct
  source 1..20: mol_type = other DNA; note = NANOG knock-in 3' ARM test primer Forword
gcaacctccccttctacgag

SEQ ID NO: 61 - DNA, 20 bp, synthetic construct
  source 1..20: mol_type = other DNA; note = NANOG knock-in 3' ARM test primer Reverse
gtgaagcacacggtagggta

SEQ ID NO: 62 - DNA, 23 bp, synthetic construct
  source 1..23: mol_type = other DNA; note = NANOG knock-in 5' ARM test primer Forword
tgtccattgctgaagcatgtaat

SEQ ID NO: 63 - DNA, 23 bp, synthetic construct
  source 1..23: mol_type = other DNA; note = NANOG knock-in 5' ARM test primer Reverse
gaagttagtagctccgcttcctg